(19) United States
(12) Patent Application Publication
(10) Pub. No.: US 2024/0352477 A1
(43) Pub. Date: Oct. 24, 2024

(54) METHOD FOR PROMOTING GROWTH OF PLANTS

(71) Applicant: ACADEMIA SINICA, Taipei City (TW)

(72) Inventors: Su-May YU, Taipei City (TW); Hsiang-Ting LEE, Kaohsiung City (TW); Tuan-Hua David HO, Taipei City (TW)

(73) Assignee: ACADEMIA SINICA, Taipei City (TW)

(21) Appl. No.: 18/645,166

(22) Filed: Apr. 24, 2024

Related U.S. Application Data
(60) Provisional application No. 63/497,834, filed on Apr. 24, 2023.

(51) Int. Cl. C12N 15/82; C12N 9/12

(52) U.S. Cl. CPC C12N 15/8261; C12N 9/12; C12N 15/8227; C12N 15/8262; C12N 15/8273; C12Y 207/11001

(57) ABSTRACT

The present invention relates to a method for promoting growth of plants. Particularly, the method of the present invention features overexpression of a calcineurin B-like (CBL) interacting protein kinase15 (CIPK15) having an isoleucine (Ile) residue at position 48 (e.g. a CIPK15 from rice) in plants which can promote growth of the plants. The present invention also relates to a recombinant construct comprising a nucleic acid encoding the CIPK15 operably linked to a promoter. The present invention further relates to a transgenic plant overexpressing the CIPK15 exhibiting improved growth compared to a non-transgenic plant counterpart.

### CROSS-REFERENCE TO RELATED APPLICATIONS

This application claims the benefit of U.S. provisional application No. 63/497,834, filed Apr. 24, 2023 under 35 U.S.C. § 119, the entire content of which is incorporated herein by reference.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 23, 2024, is named “20240423-ACA0163US-sequence listing.xml” and is 73,728 bytes in size. The sequence listing contained in this .XML file is part of the specification and is hereby incorporated by reference herein in its entirety.

### TECHNOLOGY FIELD

The present invention relates to a method for promoting growth of plants. Particularly, the method of the present invention features overexpression of a calcineurin B-like (CBL) interacting protein kinase15 (CIPK15) having an isoleucine (Ile) residue at position 48 (e.g. a CIPK15 from rice) in plants which can promote growth of the plants. The present invention also relates to a recombinant construct comprising a nucleic acid encoding the CIPK15 operably linked to a promoter. The present invention further relates to a transgenic plant overexpressing the CIPK15 exhibiting improved growth compared to a non-transgenic plant counterpart.

### BACKGROUND OF THE INVENTION

Flooding is a widespread natural disaster that can account for up to 20% of yield losses of global food production¹. Due to the slow diffusion of oxygen (O₂) in water, plant roots submerged under water suffer O₂ deficiency that impairs respiration and nutrient uptake from the soil, leading to cell damage, growth retardation and even death. Rice is the only cereal capable of growth under aquatic environments due to its activation of various anatomical, metabolic and/or morphological mechanisms to adapt to periods of hypoxia or anoxia. Such mechanisms include early production of enzymes for starch conversion to sugars for anaerobic germination and seedling development², glycolysis in the absence of O₂ through fermentative metabolism³, specific structures (e.g., aerenchyma in parenchymal tissues) that improve access to O₂⁴, gibberellin- and ethylene-promoted internode elongation to allow plants to extend above the water surface for gas exchange (e.g., deep water rice)³, and energy conservation during flooding and drought tolerance after recovery from flooding (e.g., lowland rice)⁵.

Rice is one of the most important crops in the world and is the principal food of nearly 50% of the world's population. Direct seeding, an important agricultural practice for rice, is increasingly employed by farmers in rain-fed and irrigated fields in Asia given its advantages of reduced labor, energy and water use, and production costs⁶. Underwater germination and seedling development is a prerequisite for effective direct-seeded rice cultivation. Despite tolerance of rice roots to waterlogging and partial submergence at vegetative stages, most rice varieties are extremely sensitive to hypoxia during germination and early seedling growth stages⁶. Under hypoxia, tolerant rice genotypes exhibit speedy hydrolysis of starch to sugars to fuel anaerobic metabolism, germination and coleoptile elongation, root and leaf development, and seedling emergence above the water surface^{2,7,8}. Early increases in α-amylase (αAmy) activity (a few hours after seed imbibition) and late increases in ethylene accumulation (3 days after seed sowing) are tightly associated with enhanced seedling elongation and survival of submergence-tolerant rice varieties⁹.

Among the major cereals, only rice seeds produce the αAmy needed to mobilize starch into fermentable sugars for NAD⁺ recycling and ATP generation during anaerobic germination and coleoptile development¹⁰. The sugar starvation-inducible transcription factor MYBS1 binds to and activates the αAmy promoter, with energy-sensing SNF-related protein kinase 1A (SnRK1A) upregulating MYBS1 in response to sugar starvation and hypoxia^{2,11-13}. CIPK15, a calcineurin B-like (CBL) protein-interacting Ser/Thr protein kinase induced by sugar starvation and hypoxia, activates the SnRK1A-dependent pathway to induce αAmy and alcohol dehydrogenase genes ADH1/2, and to control sugar and energy production during anaerobic germination and seedling growth^{2,7}. CIPK15 knock-out mutation in rice impairs both underwater germination and seedling growth, as well as mature plant growth in waterlogged soil².

The coleoptile is a conical plant structure and, upon exposure to O₂, it undergoes splitting, senescence, and aerenchyma development to facilitate leaf and root growth¹⁴. Lysigenous aerenchyma is formed by the development of longitudinal gaseous channels resulting from death and lysis of cortical cells in roots and of parenchyma cells in shoots¹⁵. In rice, aerenchyma is also developed in the leaf midrib, sheath and stem internodes¹⁶, providing an internal channel for O₂ transmission from shoots to root tips, which contributes to plant tolerance to waterlogged soil¹⁵. Although rice coleoptile elongation is rapid under water, root elongation is rather slow¹⁷. Active root growth occurs as coleoptiles approach the water surface where the O₂ concentration is higher^{8,9}, indicating that O₂ acquisition by the coleoptile facilitates root development.

In rice roots, aerenchyma is constitutively formed in well-drained soils and further induced upon soil waterlogging, with induction being mediated by an ethylene-reactive oxygen species (ROS)-dependent programed cell death (PCD) pathway^{15,18}. Rice produces more ethylene in its roots than maize due to higher expression under hypoxia of genes in the ethylene biosynthesis pathway, such as ACC SYNTHASE 1 (ACS1) and ACC OXIDASE 5(ACO5)¹⁹. Excess ethylene accumulation in root cortical cells promotes expression of RESPIRATORY BURST OXIDASE HOMOLOG H (RBOHH), the protein product of which converts O₂ to O₂⁻, thereby increasing H₂O₂ levels¹⁵. The resulting ROS signal further induces cell wall-degrading enzymes to trigger PCD in cortical cells to form aerenchyma^{20,21} Despite our comprehensive understanding of how aerenchyma development is regulated by ethylene under hypoxia, the mechanism connecting hypoxia-sensing to ethylene biosynthesis and signaling has remained unclear.

Hexokinases (HXKs) are dual-function proteins that not only catalyze the ATP-dependent phosphorylation of hexose in the first step of glycolysis, but also serve as a sugar sensor monitoring sugar level in cells^{22,23}. Most higher organisms express multiple HXK isoforms that are associated with various cellular membranes to exert diverse biological functions²⁴. Arabidopsis has six HXK members and rice has ten^{25,26}, but very few have been functionally characterized. In Arabidopsis, HXK1 senses glucose and integrates nutrient, light and hormone signaling networks to control growth and development²³. HXKs mediate sugar feed-back repression of genes essential for photosynthesis and starch degradation, but the underlying mechanism remains largely unknown²⁷. The voltage-dependent anion channel (VDAC) is the most abundant protein in the mitochondrial outer membrane and it is the major channel responsible for transporting ions, Ca²⁺, ADP/ATP and other metabolites in and out of these organelles²⁸. In animal cells, interaction of VDAC1 with HXKs on mitochondrial membranes plays important roles in regulating cellular energy production, metabolism and apoptosis²⁹.

The development of an aerated root system in waterlogged soil is a fundamental requirement for the flooding tolerance displayed by plants. Understanding the mechanism by which rice root growth is promoted under flooding or hypoxia is not only of fundamental scientific significance, but it is also important for breeding rice and other crops with enhanced flooding tolerance. There is also a need to provide an approach to promote growth, yield and root development of plants and enhance their tolerance to environmental stress.

### SUMMARY OF THE INVENTION

In this invention, it is unexpectedly found that overexpression of a calcineurin B-like (CBL) interacting protein kinase15 (CIPK15) in a plant can improve growth, yield and/or root development of the plant, wherein the CIPK15 has an isoleucine (Ile) residue at a position corresponding to position 48 of SEQ ID NO: 1 (e.g. a CIPK15 from rice). The transgenic plant also exhibits enhanced tolerance to environmental stress e.g. drought, salinity and flooding stresses, and/or increased ethylene production.

Therefore, in one aspect, the present invention provides a method for promoting growth, yield and/or root development of a plant, comprising
(a) transforming plant cells with a recombinant construct comprising a nucleic acid operably linked to a promoter to obtain recombinant plant cells, wherein the nucleic acid encodes a calcineurin B-like (CBL) interacting protein kinase15 (CIPK15) having an isoleucine (Ile) residue at position 48 of SEQ ID NO: 1, such that the recombinant plant cells overexpressing the CIPK15;
(b) growing the recombinant plant cells obtained in (a) to generate a plurality of transgenic plants; and
(c) selecting a transgenic plant from the plurality of transgenic plants generated in (b) that exhibits improved growth, yield and/or root development as compared with a non-transgenic plant counterpart growing under the same conditions.

In some embodiments, the CIPK15 is a CIPK15 from rice.

In some embodiments, the CIPK15 is a CIPK15 from barley, wheat, rye, oat, millet, corn, bamboo, sugarcane, onion, leek, ginger, stiff brome, millet, green foxtail, sorghum, Kans grass or Hall's panicgrass, having an isoleucine (Ile) substituent at a position corresponding to position 48 of SEQ ID NO: 1.

In some embodiments, the CIPK15 comprises
(a) an amino acid sequence selected from the group consisting of SEQ ID NO: 1-4 and 19-32; or
(b) an amino acid sequence having a sequence identity of at least 80% with the amino acid sequence of (a), and having an isoleucine (Ile) residue at position 48 of SEQ ID NO: 1.

In some embodiments, the promoter is a root-specific promoter.

In some embodiments, the promoter is a native rice CIPK15 promoter.

In some embodiments, the transgenic plant exhibits improved growth, yield and/or root development as compared with a non-transgenic plant counterpart under aerobic or hypoxic conditions.

In some embodiments, the transgenic plant exhibits increased plant height, elongated coleoptiles and shoot, higher grain yield, longer roots, larger root radius, and/or more aerenchyma.

In some embodiments, the transgenic plant further exhibits enhanced tolerance to environmental stress.

In some embodiments, the environmental stress includes drought, salinity and/or flooding stresses.

In some embodiments, the transgenic plant further exhibits enhanced ethylene production.

In some embodiments, the transgenic plant is a monocot plant.

In some embodiments, the transgenic plant is rice, barley, wheat, rye, oat, corn, bamboo, sugarcane, onion, leek, ginger, stiff brome, millet, green foxtail, sorghum, cutgrasses, Kans grass or Hall's panicgrass.

In some embodiments, the transgenic plant is a dicot plant.

In some embodiments, the transgenic plant is Arabidopsis, soybean, peanut, sunflower, safflower, cotton, tobacco, tomato, pea, chickpea, pigeon pea or potato.

In another aspect, the present invention provides a recombinant construct comprising a nucleic acid encoding CIPK15 operably linked to a promoter, wherein the CIPK15 has an isoleucine (Ile) residue at a position corresponding to position 48 of SEQ ID NO: 1.

In some embodiments, the CIPK15 is a CIPK15 from rice.

In some embodiments, the CIPK15 is a CIPK15 from barley, wheat, rye, oat, millet, corn, bamboo, sugarcane, onion, leek, ginger, stiff brome, millet, green foxtail, sorghum, Kans grass or Hall's panicgrass, having an isoleucine (Ile) substituent at a position corresponding to position 48 of SEQ ID NO: 1.

In some embodiments, the CIPK15 comprises
(a) an amino acid sequence selected from the group consisting of SEQ ID NOs: 1-4 and 19-32; or
(b) an amino acid sequence having a sequence identity of at least 80% with the amino acid sequence of (b), and having an isoleucine (Ile) residue at position 48 of SEQ ID NO: 1.

In some embodiments, the promoter is a root-specific promoter.

In some embodiments, the promoter is a native rice CIPK15 promoter.

In a further aspect, the present invention provides a transgenic plant comprising an exeogenous nucleic acid operably linked to a promoter, wherein the exogenous nucleic acid (a transgene) encodes CIPK15. In some embodiments, the transgenic plant is generated by transformation with a recombinant construct encoding CIPK15 as described herein.

In some embodiments, the transgenic plant exhibits improved growth, yield and/or root development as compared with a non-transgenic plant counterpart.

In some embodiments, the transgenic plant exhibits improved growth, yield and/or root development as compared with a non-transgenic plant counterpart under aerobic or hypoxic conditions.

In some embodiments, the transgenic plant exhibits increased plant height, elongated coleoptiles and shoot, higher grain yield, longer roots, larger root radius, and/or more aerenchyma.

In some embodiments, the transgenic plant further exhibits enhanced tolerance to environmental stress.

In some embodiments, the environmental stress includes drought, salinity and/or flooding stresses.

In some embodiments, the transgenic plant further exhibits enhanced ethylene production.

In some embodiments, the transgenic plant is a monocot plant.

In some embodiments, the transgenic plant is rice, barley, wheat, rye, oat, corn, bamboo, sugarcane, onion, leek, ginger, stiff brome, millet, green foxtail, sorghum, cutgrasses, Kans grass or Hall's panicgrass.

In some embodiments, the transgenic plant is a dicot plant.

In some embodiments, the transgenic plant is Arabidopsis, soybean, peanut, sunflower, safflower, cotton, tobacco, tomato, pea, chickpea, pigeon pea or potato.

Also provided is a gene-edited plant having a genome comprising a nucleic acid encoding a CIPK15 having an isoleucine (Ile) substituent at a position corresponding to position 48 of SEQ ID NO: 1.

In particular, the plant, before genome edition, originally has a natural gene encoding a CIPK15 having an amino acid residue other than isoleucine (Ile) at a position corresponding to position 48 of SEQ ID NO: 1 in its genome, and after genome edition, the gene is modified to encode a mutant CIPK15 having an isoleucine (Ile) substituent at the position corresponding to position 48 of SEQ ID NO: 1 in its genome. In some examples, the amino acid residue other than isoleucine (Ile) at a position corresponding to position 48 of SEQ ID NO: 1 is valine (Val) or alanine (Ala).

In some embodiments, the mutant CIPK15 comprises (a) an amino acid sequence selected from the group consisting of SEQ ID NOs:19-32 or (b) an amino acid sequence having a sequence identity of at least 80% with the amino acid sequence of (a) and having an isoleucine (Ile) residue at position 48 of SEQ ID NO: 1.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIGS. 1a to 1m. CIPK15 regulates germination, root growth and aerenchyma development, in rice under hypoxia via multiple pathways. (FIG. 1a) Rice seeds germinated under hypoxia for 8 and 9 days. (FIG. 1b) Transgenic rice carrying CIPK15:GUS were germinated under aerobic or hypoxic conditions for various lengths of time and then stained for GUS expression. Scale bars=0.5 cm. Abbreviation: C: coleoptile; L: leaf; S: shoot; R: root. (FIGS. 1c to 1h) Two-day-old seedlings of WT, cipk15 and CIPK15-overexpressing (CIPK15-Ox) lines were grown under aerobic or hypoxic conditions for 12 days. (FIG. 1c, FIG. 1f) Root morphology. (FIG. 1d, FIG. 1g) Cross-sections from 2 cm above seminal root tips and stained with toluidine blue. Aerenchyma is indicated by red dots. Scale bar=50 μm. (FIG. 1e, FIG. 1h) Occupancy (in %) of aerenchyma area, calculated by dividing total aerenchyma area by total root area from cross-sections taken at various distances from the root tips of seminal roots (n≥10). (FIG. 1i) CIPK15 transcript abundances, as determined by RNAseq and compared between WT and the cipk15 mutant under hypoxic and aerobic conditions. The color scale of log 2 fold-change ranges from −2 to 2 (for the ethylene- and auxin-dependent pathways) or −15 to 15 (for the SnRK1A-dependent pathway), with red and blue representing higher and lower transcript levels, respectively. (FIGS. 1j to 1m) WT and cipk15 mutant seedlings hydroponically cultured with or without 10 μM ethephon in a hypoxic chamber (n=5). (FIG. 1j) Morphologies of 7-day-old seedlings. Cross-sections were taken at various distances from seminal root tips and stained with toluidine blue. (FIG. 1k) Quantitative data of primary root length. Values represent mean±SD (n≥5). (FIG. 11) Cross-sections at 1.5 cm from seminal root tips. (FIG. 1m) Quantification (in %) of total aerenchyma area divided by total root area from root cross-sections. nd, non-detectable. Values represent mean±SD.

FIGS. 2a to 2d. Sugar starvation and hypoxia induce CIPK15-mediated regulation of genes essential for coleoptile and root development under hypoxia and ultimately promote grain yield. (FIG. 2a) WT (left panel) and cipk15 (right panel) lines germinated and then allowed grow for 7 days under hypoxia. (FIG. 2b) qRT-PCR analysis of mRNAs extracted from the coleoptiles and roots described in (FIG. 2a). Values represent mean±SD (n=3). nd: non-detectable. Rice protoplasts were co-transfected with effector and reporter plasmids, and then cultured in medium with or without glucose with shaking (+O₂) or without shaking (—O₂). Total proteins were extracted from the protoplasts and assayed for luciferase activity. (FIG. 2c) CIPK15 regulates multiple pathways responsible for coleoptile and root development under hypoxia. (FIG. 2d) Total grain weight of plants grown in an open field or rainout shelter with regular irrigation. Yield was collected from individual plants (n≥10). The numbers above the box plot represent percentages of grain weight for each line relative to WT. The numbers below the box plot are average grain weights per plant of each line. Statistical analysis was carried out by ANOVA with a post-hoc Tukey HSD at p<0.05.

FIGS. 3a to 3g. HXK6 and VDAC1 interact with CIPK15 on mitochondria and suppress CIPK15-regulated promoters. (FIG. 3a) BiFC assay in which rice protoplasts were co-transfected with CaWV35S:cYFP-CIPK15 or CaWV-nYFP (negative control) and CaMV35S:HXK5/6-nYFP or CaMV35S:cYFP (negative control). (FIG. 3b, FIG. 3c, FIG. 3f) Rice protoplasts were transfected with plasmids, cultured in medium containing 400 mM glucose with shaking for 12 h, and then examined under confocal microscopy. Scale bar=10 m. C: cytoplasm; Mt: mitochondria. (FIG. 3b) Rice protoplasts were co-transfected with Ubi:CIPK15-mTagBFP and Ubi:HXK6-mCherry and stained with the mitochondrial marker MitoTracker® Green FM. (FIG. 3c) Rice protoplasts were co-transfected with Ubi:CIPK15-mCherry and Ubi:GFP, Ubi:HXK6-GFP, or Ubi:HXK6(Ri) constructs. (FIG. 3d, FIG. 3g) Rice protoplasts were co-transfected with effector and reporter plasmids, cultured in medium contained 400 mM glucose with shaking, and assayed for luciferase activity. (FIG. 3e) Rice protoplasts were co-transfected with Ubi:VDAC1-GFP, Ubi:CIPK15-HA and Ubi:HXK6-GFP or Ubi:HXK6-mCherry, cultured in medium containing 400 mM glucose with shaking for 12 h, and total proteins were extracted and pulled down using GFP trap®. Anti-GFP, anti-HA and anti-mRFP antibodies were used to detect HXK6-GFP or VDAC1-GFP, CIPK15-HA, and HXK6-mCherry, respectively. We noticed that some HXK6-GFP in the input proteins extracted from cells co-transfected with HXK6-GFP and CIPK15-HA was degraded to a protein with similar molecular weight to VDAC1-GFP. (FIG. 3f) Rice protoplasts were co-transfected with Ubi:CIPK15-mCherry, Ubi:HXK6-mCherry and Ubi:VDAC1-GFP.

FIGS. 4a to 4k. Binding of glucose to HXK6 is necessary to suppress CIPK15 activity and inhibit aerenchyma and root development. (FIG. 4a) Schematic showing the relative positions of the functional domains and amino acids in HXK6. Mito: mitochondria. Glc: glucose. (FIG. 4b) Constructs of effector and reporter plasmids. (FIG. 4c, FIG. 4d) Rice protoplasts were co-transfected with effector and reporter plasmids, cultured in medium with or without glucose for 12 h without shaking (hypoxic). Total proteins were then extracted from the protoplasts and assayed for luciferase activity. (FIGS. 4e to 4j) Seedlings of TNG67 WT and hxk6 mutants were grown in medium under aerobic or hypoxic conditions for 7 days. (FIG. 4e) Morphology of the rice seedlings. Scale bar=2 cm. (FIG. 4f) Primary root lengths of the seedlings, with values representing mean±SD (n≥5). (FIG. 4g) Primary roots were cross-sectioned 1.5 cm above root tips and stained with toluidine blue. Aerenchyma is indicated by red dots. Scale bar=50 m. (FIG. 4h) Occupancy (%) of aerenchyma area, as calculated by dividing aerenchyma area by total root area in cross-sections (n=5). (FIG. 4i) Diagram showing the position of the T-DNA insertion −42 bp upstream of the transcription start site (+1) of HXK6 in the rice genome. (FIG. 4j) Total RNAs were isolated from hxk6 roots and subjected to qRT-PCR analysis. (FIG. 4k) Transgenic rice calli carrying Ubi:HXK6-mCherry and TNG67 WT were cultured under aerobic conditions for 2 days, before isolating the total RNAs and subjecting them to qRT-PCR analysis. Values represent mean±SD (n=3).

FIGS. 5a to 5f. The NAF domain is necessary for CIPK15 activity, mitochondria localization and interaction with HXK6. (FIG. 5a) Schematic showing the relative positions of the functional domains and amino acids in CIPK15. (FIG. 5b) Rice protoplasts were co-transfected with effector and reporter constructs, and then cultured in medium with glucose for 12 h without shaking (hypoxic). Endogenous ACS1 mRNA (reporter) levels were then assayed by means of qRT-PCR analysis. (FIG. 5c) Identification of the phosphorylated residue in CIPK15 by LC MS/MS analysis. The peptide sequence above the spectrum illustrates the positions of phosphorylated peptides in CIPK15. The MS/MS spectra that yielded the highest Mascot score for matching b and y ions identified phosphorylated peptides. The assigned b and y ions are shown in red and orange fonts, respectively. Ions showing neutral loss of a phosphate moiety (H₃PO₄) are labeled with −98 (*). The phosphorylated Thrl71 in the peptide sequence is highlighted in red font. (FIG. 5d) Rice protoplasts were co-transfected with effector and reporter plasmids, cultured in medium without glucose for 12 h with shaking (aerobic). Total proteins were then extracted from the protoplasts and assayed for luciferase activity. (FIG. 5e) Rice protoplasts were transfected with CIPK15 mutant-mCherry constructs alone or with HXK6 mutant-GFP constructs and cultured in medium containing glucose with shaking (aerobic) for 12 h. Cells were stained with the mitochondrial marker MitoTracker® Green FM and examined under confocal microscopy. C: cytosol, N: nucleus, Mt: mitochondria. Scale bar=10 μm. (FIG. 5f) Rice protoplasts were co-transfected with effector and reporter constructs, and then cultured in medium without glucose and shaking (aerobic) for 12 h. Total proteins were then extracted from the protoplasts and assayed for luciferase activity.

FIGS. 6a to 6c. The role of the CIPK15-HXK6-VDAC1 interaction that regulates hypoxia tolerance in rice. Details of the model are described in the text. (FIG. 6a) Regulation of CIPK15 activity by HXK6-VDAC1 and glucose. (FIG. 6b) Function of the CIPK15 NAF domain. (FIG. 6c) Regulation of CIPK15 function in root growth by O₂.

FIGS. 7a to 7c. CIPK15 knockout mutant and inducible overexpression lines. (FIG. 7a) Diagram showing retrotransposon Tos17 inserted at the coding region 639 bp downstream of the translation ignition codon ATG of CIPK15 in the mutant cipk15-1 rice line. (FIG. 7b) Diagram showing the structure and molecular action of the β-estradiol-inducible expression system for overexpressing CIPK15. (FIG. 7c) Two-day-old germinated seedlings of WT and transgenic rice carrying XVE:CIPK15-dHA were grown in medium containing 5 μM β-estradiol for an additional 3 days. Total proteins were extracted from roots and subjected to protein gel blot analysis using anti-HA antibody. Protein loading is shown by Ponceau S staining. +/− indicates medium with or without β-estradiol.

FIG. 8. Leaf and root development are slower in the cipk15 mutant relative to WT under hypoxia. Rice seeds were germinated on agar medium under aerobic or submerged/hypoxic conditions for 8 and 9 days. The seedlings in yellow boxes are also presented in FIG. 1a.

FIGS. 9a to 9d. CIPK15 regulates root growth and aerenchyma development in rice. Two-day-old seedlings of WT, cipk15 and CIPK15-Ox lines were grown under aerobic or hypoxic conditions for 12 days. (FIG. 9a, FIG. 9c) Growth curves of seminal roots under aerobic and hypoxic conditions. Values of seminal root lengths represent mean±SD (n≥10). (FIG. 9b, FIG. 9d) Radii of root cross-sections taken 2 cm above seminal root tips (n=5).

FIGS. 10a to 10c. CIPK15 upregulates genes involved in root development via ethylene- and auxin-dependent pathways under hypoxia. (FIG. 10a, FIG. 10b) WT and cipk15 lines were germinated for 2 days and transferred to glass tubes containing 1/2X MS medium for another 12 days. Total RNAs were isolated from roots of (FIG. 10a) WT and cipk15 mutant, and (FIG. 10b) WT and CIPK15-Ox lines, and subjected to qRT-PCR analysis using various gene-specific primers. Values represent mean±SD (n=3). (FIG. 10c) Rice protoplasts were co-transfected with effector and reporter plasmids, cultured in medium containing 400 mM glucose for 12 h without shaking (hypoxic condition), and assayed for GUS activity.

FIGS. 11a to 11d. Expression of CIPK15 in knockout mutant and inducible overexpression lines. (FIG. 11a) Total RNAs were extracted from 5-day-old seedlings of WT and cipk15 lines grown under aerobic or submerged conditions and subjected to RNAseq analysis. Gene annotation was performed in IGV software (https://software.broadinstitute.org/software/igv/), and CPM counts indicated no CIPK15 mRNA accumulation in the cipk15 line under either aerobic or hypoxic conditions. (FIG. 11b) Diagram showing the differences in CIPK14 and CIPK15 amino acid sequences. Asterisks represent translation stop codons. (FIG. 11c) CIPK15 mutant constructs. (FIG. 11d) Rice protoplasts were co-transfected with CIPK15 mutants and ACS1:LUC constructs and then cultured in medium without glucose for 12 h. Total proteins were extracted from the protoplasts and assayed for luciferase activity. Error bars indicate the SE from three independent experiments. Statistical analysis by ANOVA with post-hoc Tukey HSD test at P<0.05.

FIG. 12. CIPK15 expression is mainly induced in roots of CIPK15:CIPK15 transgenic plants. Seedlings of WT (TNG67) and CIPK15: CIPK15 transgenic lines were grown in 1/2X MS medium for 7 days. Shoots and roots were then harvested separately for qRT-PCR analysis. Values represent mean±SD (n=3). Statistical analysis by ANOVA with post-hoc Tukey HSD at p<0.05.

FIGS. 13a to 13b. CIPK15 and HXK5/6 interact and localize at mitochondria under aerobic and hypoxic conditions. (FIG. 13a) Rice protoplasts were co-transfected with Ubi:CIPK15-mCherry and Ubi:HXK5-GFP, Ubi:HX6-GFP or Ubi:GFP (as negative control), before being cultured in medium containing 400 mM glucose with shaking (aerobic) for 12 h. Then, total proteins were extracted and pulled down using RFP trap®. Anti-RFP and anti-GFP antibodies were used to detect co-immunoprecipitated (co-IP) CIPK15-mCherry and HXK5-GFP or GFP, respectively. (FIG. 13b) Rice protoplasts were transfected with Ubi:CIPK15-mCherry, Ubi:HXK5-mCherry or Ubi:HXK6-mCherry and stained with the mitochondrial marker MitoTracker® Green FM, before being examined under confocal microscopy. T: Transmission image. Scale bar=10 m.

FIG. 14. HXK5/6 suppresses the CIPK15-regulated ACS1 promoter and mRNA accumulation. Rice protoplasts were co-transfected with effector and reporter plasmids, cultured in medium containing 400 mM glucose with shaking (aerobic) or without shaking (hypoxic), and then assayed for luciferase activity and mRNA levels.

FIG. 15. HXK6 lacking a mitochondrial targeting signal interacts with CIPK15 in the cytoplasm. Rice protoplasts were co-transfected with Ubi: CIPK15-mCherry and Ubi: GFP, Ubi:HXK6-GFP, or Ubi-HXK6ΔTS-GFP constructs, and then examined under confocal microscopy. C: cytoplasm; N: nucleus; Mt: mitochondria. Scale bar=10 m.

FIGS. 16a to 16b. HXK5/6 and VDAC1 are downregulated under hypoxia. Total RNAs were extracted from 5-day-old seedlings of Nipponbare WT rice grown under aerobic or hypoxic conditions and subjected to RNAseq analysis. Gene annotation was performed in IGV software (https://software.broadinstitute.org/software/igv/) and CPM counts indicate the extent of VDAC1 mRNA accumulation under aerobic or hypoxic conditions (n=3). (FIG. 16a) Accumulation of HXK5/6 transcripts. (FIG. 16b) Accumulation of VDAC1 transcripts.

FIGS. 17a to 17b. CIPK15 protein is more stable under hypoxia. Two-day-old rice seedlings were grown under aerobic or hypoxic conditions for 2 days and treated with or without 100 μM cycloheximide (CHX) for an additional 1, 2 or 4 h. (FIG. 17a) Seedlings treated with CHX for 1 or 2 h were harvested for protein extraction for immunoblotting assay using anti-CIPK15 antibody. (FIG. 17b) Seedlings treated with CHX for 2 or 4 h were harvested for RNA extraction to undergo qRT-PCR analysis using primers targeting CIPK14/15 mRNA.

FIGS. 18a to 18g. OsCIPK15 improves root growth and aerenchyma development in Setaria viridis. Seedlings of WT, OsCIPK15-7 and OsCIPK15-8 were germinated on ½ MS agar medium and grown under aerobic or hypoxic treatments for 14 days. (FIG. 18a) Morphology of rice seedlings. Bar=1 cm. (FIG. 18b) Cross-sections from 1 cm above seminal root tips and stained with toluidine blue. Aerenchyma is indicated by red dots. Bar=50 mm(FIG. 18c) Quantitative data of primary root length. Values represent mean±SD (n≥5). Occupancy (in %) of aerenchyma area, calculated by dividing total aerenchyma area by total root area from cross-sections taken at various distances from the root tips of seminal roots (n≥5). (FIG. 18d) Total RNA were isolated from 14-days-old seedlings of WT, OsCIPK15-7 and OsCIPK15-8 lines and subjected to qRT-PCR analyses using gene specific primers. Values were means±SD (n=3). (FIG. 18e) Seven-day-old seedlings of WT and Sev(OsCIPK15-7) lines were germinated under aerobic conditions for 2 days and transferred to aerobic or hypoxic conditions for another 7 days. Cross-sections at 1 cm above first nodal root tips were stained with acriflavine for lignin. Upper two panels are entire cross sections, and lower two panels are parts of enlarged peripheral cell layers. Arrowhead marks the increased lignification in walls of cortex cells (under aerobiosis) and sclerenchyma (under hypoxia). F: fluorescence image of lignin. T: transmission image of tissues. Bar=100 mm. C: cortex, En: endodermis, Ep: epidermis, S: stele, Sc: sclerenchyma. (FIG. 18f) Total RNAs were extracted from 10-day-old seedlings and subjected to qRT-PCR analyses using gene-specific primers. A: aerobic. H: hypoxic. n=3. (FIG. 18g) Rice protoplast transient expression assays. Effector constructs. Reporter constructs and luciferase activity.

FIG. 19. Sequence alignment of OsCIPK15 and the orthologue proteins. Protein sequences and orthologues prediction were downloaded from Ensemble plants database (https://plants.ensembl.org/). MEGA7 software is used for sequence alignment. The amino acids marked in color indicates 100% similarity among various species. The frames indicate the positions for distinguished OsCIPK14/15 (I48, P292), and the conserved residues for NAF domain (NAF, L328, F332). OsCIPK14 (Oryza sativa, SEQ ID NO: 33). OsCIPK15 (Oryza sativa, SEQ ID NO: 1). SevCIPK15 (Setaria viridis, SEQ ID NO: 7). ZmCIPK15 (Zea mays, SEQ ID NO: 14).

### DETAILED DESCRIPTION OF THE INVENTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

As used herein, the term “comprise” or “comprising” is generally used in the sense of include/including which means permitting the presence of one or more features, ingredients or components. The term “comprise” or “comprising” encompasses the term “consists” or “consisting of.”

As used herein, the term “about” or “approximately” refers to a degree of acceptable deviation that will be understood by persons of ordinary skill in the art, which may vary to some extent depending on the context in which it is used. In general, “about” or “approximately” may mean a numeric value having a range of ±10% around the cited value

As used herein, the term “polypeptide” or proteins refers to a polymer composed of amino acid residues linked via peptide bonds.

As used herein, the term “corresponding to” refers to a residue at the enumerated position in a protein or peptide, or a residue that is analogous, homologous, or equivalent in position to the enumerated residue in a different protein or peptide. Determining corresponding amino acid residues can be done by aligning the sequences and identifying residues that like across from one another in the resultant alignment. Namely, the residue number or position of a given amino acid sequence is designated with respect to the reference sequence rather than by the actual numerical position of that residue within the given amino acid sequence. The same corresponding rule is applicable to nucleotides in polynucleotide sequences.

As used herein, the term “substantially identical” refers to two sequences having 80% or more, preferably 85% or more, more preferably 90% or more, even more preferably 95% or more homology.

As used herein, the term “polynucleotide” or “nucleic acid” refers to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. The term “nucleic acid” typically refers to large polynucleotides. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

As used herein, the term “complementary” refers to the topological compatibility or matching together of interacting surfaces of two polynucleotides. Thus, the two molecules can be described as complementary, and furthermore the contact surface characteristics are complementary to each other. A first polynucleotide is complementary to a second polynucleotide if the nucleotide sequence of the first polynucleotide is identical to the nucleotide sequence of the polynucleotide binding partner of the second polynucleotide. Thus, the polynucleotide whose sequence 5′-TATAC-3′ is complementary to a polynucleotide whose sequence is 5′-GTATA-3′.”

As used herein, the term “encoding” refers to the inherent property of specific sequences of nucleotides in a polynucleotide (e.g., a gene, a cDNA, or an mRNA) to serve as templates for synthesis of other polymers and macromolecules in biological processes having either a defined sequence of nucleotides (i.e., rRNA, tRNA and mRNA) or a defined sequence of amino acids and the biological properties resulting therefrom. Therefore, a gene encodes a protein if transcription and translation of mRNA produced by that gene produces the protein in a cell or other biological system. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “overexpression” can refer to the production of a gene product in transgenic plants that exceeds levels of production in non-transgenic plants, including but not limited to constitutive or induced expression.

As used herein, the term “transgenic plant” or “transgenic line” refers to a plant that contains a recombinant nucleotide sequence that encodes a gene i.e. a transgene. The transgenic plant can be grown from a recombinant cell.

As used herein, the term “genome editing” or “gene-editing” can refer to introducing a targeted variation and/or mutation into the nucleotide sequence of plant cells, including knocking-out or knocking-in a specific gene, or introducing a variation into a coding DNA sequence or a non-coding DNA sequence that produces no protein. For the purpose of the present disclosure, the genome editing may mean introducing a mutation or change (e.g. an isoleucine (Ile) substituent at a position corresponding to position 48 of SEQ ID NO: 1) using a Cas protein and a guide RNA, for example.

### I. Calcineurin B-Like (CBL) Interacting Protein Kinase15 (CIPK15)

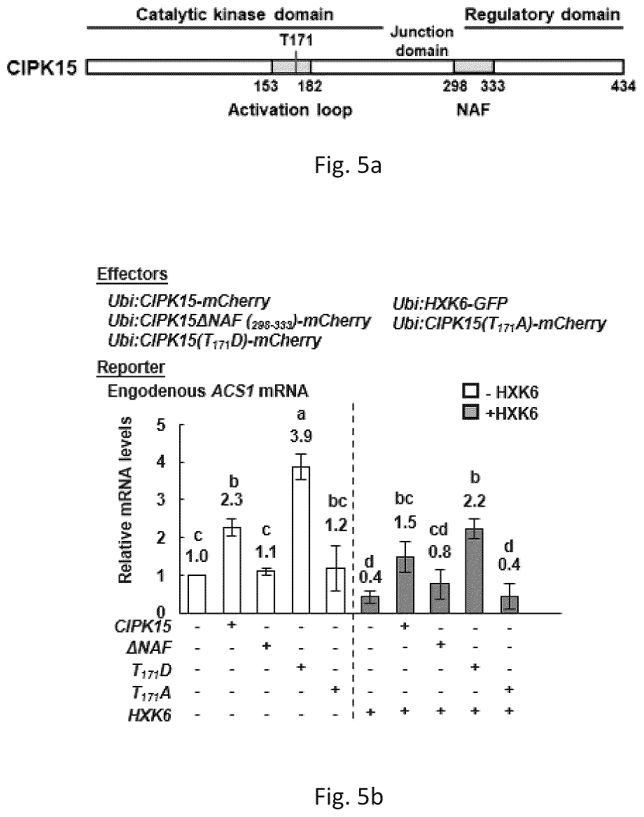

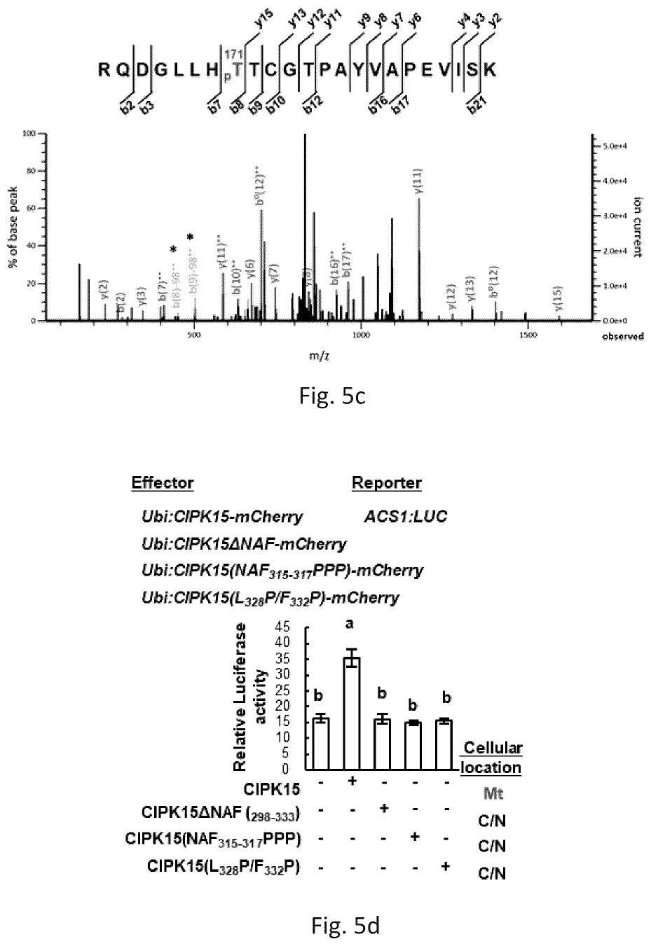

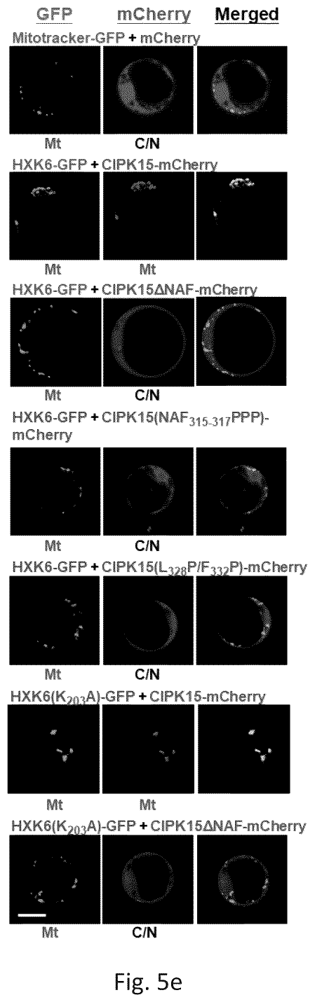

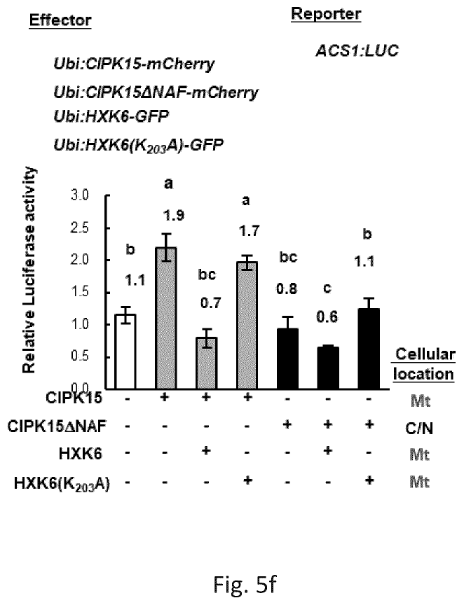

CIPK15 is known as a plant-specific Ser-Thr protein kinase containing a conserved NAF (Asn-Ala-Phe) domain, which is critical for the interaction with calcineurin B-like proteins. CIPK15 can be found in a variety of plants. CIPK15 comprises a total of 400 to 450 amino acid residues in length. CIPK15 comprises from N-terminal to C-terminal a catalytic kinase domain, a junction domain and a regulatory domain, wherein the catalytic kinase domain includes an activation loop at a region from position 153 to 182 with a threonine (T) at position 171, and the regulatory domain includes a NAF (Asn-Ala-Phe) domain at a region from position 298 to 333, with respect to SEQ ID NO: 1. See FIG. 5a.

Specifically, a native CIPK15 from rice may have the amino acid sequence of SEQ ID NO: 1 (OsCIPK15, Oryza sativa), SEQ ID NO: 2 (ObCIPK15, Oryza barthii), SEQ ID NO: 3 (OgCIPK15, Oryza glaberrima) or SEQ ID NO: 4 (OrCIPK15, Oryza rufipogon), wherein the amino acid residue at position 48 is isoleucine (Ile 48). A native CIPK15 from stiff brome may have the amino acid sequence of SEQ ID NO: 5 (BdCIPK15, Brachypodium distachyon), a native CIPK15 from millet may have the amino acid sequence of SEQ ID NO: 6 (SiCIPK15, Setaria Italica), a native CIPK15 from green foxtail may have the amino acid sequence of SEQ ID NO: 7 (SvCIPK15, Setaria viridis), a native CIPK15 from sorghum may have the amino acid sequence of SEQ ID NO: 8 (SbCIPK15, Sorghum bicolor), and a native CIPK15 from barley may have the amino acid sequence of SEQ ID NO: 9 (HvCIPK15, Hordeum vulgare), wherein the amino acid residue at position 48 is valine (Val 48). A native CIPK15 from wheat may have the amino acid sequence of SEQ ID NO: 10 (TaCIPK15, Triticum aestivum), SEQ ID NO: 11 (TridCIPK15, Triticum dicoccoides), or SEQ ID NO: 12 (Tritd, Triticum turgidum), wherein the amino acid residue at position 53 is valine (Val 53) which is also called Val 48 since the position Val 53 corresponds to position 48 with respect to SEQ ID NO: 1 because of four amino acid residues at N-terminal of SEQ ID NO: 10-12. A native CIPK15 from wheat may have the amino acid sequence of SEQ ID NO: 13 (TuGCIPk15, Triticum Urartu), wherein the amino acid residue at position 48 is valine (Val 48). A native CIPK15 from corn may have the amino acid sequence of SEQ ID NO: 14 (ZmCIPK15, Zea mays), a native CIPK15 from cutgrasses may have the amino acid sequence of SEQ ID NO: 15 LpCIPK15 (Leersia perrieri), a native CIPK15 from Kans grass may have the amino acid sequence of SEQ ID NO: 16 (SsCIPK15, Saccharum spontaneum), a native CIPK15 from Hall's panicgrass may have the amino acid sequence of SEQ ID NO: 17 (PhCIPK15, Panicum hallii), and a native CIPK15 from rye may have the amino acid sequence of SEQ ID NO: 18 (ScCIPK15, Secale cereal), wherein the amino acid residue at position 48 is valine (Val 48).

| Name | Plant | Scientific name | SEQ ID NO | features |
|---|---|---|---|---|
| OsCIPK15 | rice | Oryza sativa | SEQ ID NO: 1 | Ile 48 |
| ObCIPK15 | rice | Oryza barthii | SEQ ID NO: 2 | Ile 48 |
| OgCIPK15 | rice | Oryza glaberrima | SEQ ID NO: 3 | Ile 48 |
| OrCIPK15 | rice | Oryza rufipogon | SEQ ID NO: 4 | Ile 48 |
| BdCIPK15 | stiff brome | Brachypodium | SEQ ID NO: 5 | Val 48 |
| | | distachyon | | |
| SiCIPK15 | millet | Setaria Italica | SEQ ID NO: 6 | Val 48 |
| SevCIPK15 | green foxtail | Setaria viridis | SEQ ID NO: 7 | Val 48 |
| SbCIPK15 | sorghum | Sorghum bicolor | SEQ ID NO: 8 | Val 48 |
| HvCIPK15 | barley | Hordeum vulgare | SEQ ID NO: 9 | Val 48 |
| TaCIPK15 | wheat | Triticum aestivum | SEQ ID NO: 10 | Val 53 |
| TridCIPK15 | wheat | Triticum dicoccoides | SEQ ID NO: 11 | Val 53 |
| Tritd CIPK15 | wheat | Triticum turgidum | SEQ ID NO: 12 | Val 53 |
| TuGCIPK15 | wheat | Triticum Urartu | SEQ ID NO: 13 | Val 48 |
| ZmCIPK15 | corn | Zea mays | SEQ ID NO: 14 | Val 48 |
| LpCIPK15 | cutgrasses | Leersia perrieri | SEQ ID NO: 15 | Ala 48 |
| SsCIPK15 | Kans grass | Saccharum spontaneum | SEQ ID NO: 16 | Val 48 |
| PhCIPK15 | Hall's | Panicum hallii | SEQ ID NO: 17 | Val 48 |
| | panicgrass | | | |
| ScCIPK15 | rye | Secale cereal | SEQ ID NO: 18 | Val 48 |

As used herein, the term “CIPK15,” unless indicated otherwise, refers to “CIPK15 with Ile 48” namely a CIPK15 from a plant species having isoleucine at a position corresponding to position 48 of SEQ ID NO: 1.

In some embodiments, CIPK15 as described herein can be a native CIPK15 from rice where the amino acid residue at position 48 is isoleucine (Ile 48) in nature. Examples of CIPK15 from rice may have the amino acid sequence of SEQ ID NO: 1, 2, 3 or 4.

In some embodiments, CIPK15 as described herein can be a CIPK15 from other plant species with modification where the amino acid residue at a position corresponding to position 48 with respect to SEQ ID NO: 1 is changed to isoleucine (Ile). Examples of modified CIPK15 may have the amino acid sequence ofany of SEQ ID NO: 19-32 which are identical to SEQ ID NO: 5-18, respectively, except that the amino acid residue at a position corresponding to position 48 with respect to SEQ ID NO: 1 is changed to isoleucine (Ile). CIPK15 as described herein can also include those comprising an amino acid sequence which has Ile 48 and are substantially identical to the amino acid sequences constituting CIPK15 as described herein. Specifically, CIPK15 as described herein may include an amino acid sequence with Ile 48, having at least 80% (e.g. 82%, 84%, 85%, 86%, 88%, 90%, 92%, 94%, 95%, 96%, 98%, or 99%) identity to SEQ ID NO: 1-4 and 19-32.

| Mutant CIPK15 | Plant | Origin | SEQ ID NO |
|---|---|---|---|
| BdCIPK15 (V48I) ¹ | stiff brome | Brachypodium distachyon | SEQ ID NO: 19 |
| SiCIPK15 (V48I) ¹ | millet | Setaria Italica | SEQ ID NO: 20 |
| SevCIPK15 (V48I) ¹ | green foxtail | Setaria viridis | SEQ ID NO: 21 |
| SbCIPK15 (V48I) ¹ | sorghum | Sorghum bicolor | SEQ ID NO: 22 |
| HvCIPK15 (V48I) ¹ | barley | Hordeum vulgare | SEQ ID NO: 23 |
| TaCIPK15 (V53I) ² | wheat | Triticum aestivum | SEQ ID NO: 24 |
| TdCIPK15 (V53I) ² | wheat | Triticum dicoccoides | SEQ ID NO: 25 |
| TtCIPK15 (V53I) ² | wheat | Triticum turgidum | SEQ ID NO: 26 |
| TuCIPK15 (V48I) ¹ | wheat | Triticum Urartu | SEQ ID NO: 27 |
| ZmCIPK15 (V48I) ¹ | corn | Zea mays | SEQ ID NO: 28 |
| LpCIPK15 (A48I) ¹ | cutgrasses | Leersia perrieri | SEQ ID NO: 29 |
| SsCIPK15 (V48I) ¹ | Kans grass | Saccharum spontaneum | SEQ ID NO: 30 |
| PhCIPK15 (V48I) ¹ | Hall's panicgrass | Panicum hallii | SEQ ID NO: 31 |
| ScCIPK15 (V48I) ¹ | rye | Secale cereal | SEQ ID NO: 32 |

¹ Valine (V) or Alanine (A) at position 48 is substituted with isoleucine (I)
² Valine (V) at position 53 is substituted with isoleucine (I)

It is understandable a polypeptide may have a limited number of changes or modifications that may be made within a certain portion of the polypeptide irrelevant to its activity or function and still result in a molecule with an acceptable level of equivalent biological activity or function. Modifications and changes may be made in the structure of such polypeptides and still obtain a molecule having similar or desirable characteristics. For example, certain amino acids may be substituted for other amino acids in the peptide/polypeptide structure (other than the conserved region) without appreciable loss of activity. Amino acid substitutions are generally based on the relative similarity of the amino acid side-chain substituents, for example, their hydrophobicity, hydrophilicity, charge, size, and the like. For example, arginine (Arg), lysine (Lys), and histidine (His) are all positively charged residues; and alanine (Ala), glycine (Gly) and serine (Ser) are all in a similar size. Therefore, based upon these considerations, arginine (Arg), lysine (Lys) and histidine (His); and alanine (Ala), glycine (Gly) and serine (Ser) may be defined as biologically functional equivalents. One can readily design and prepare recombinant genes for microbial expression of polypeptides having equivalent amino acid residues.

To determine the percent identity of two amino acid sequences, the sequences are aligned for optimal comparison purposes (e.g., gaps can be introduced in the sequence of a first amino acid sequence for optimal alignment with a second amino acid sequence). In calculating percent identity, typically exact matches are counted. The determination of percent homology or identity between two sequences can be accomplished using a mathematical algorithm known in the art, such as BLAST and Gapped BLAST programs, the NBLAST and XBLAST programs, or the ALIGN program.

### II. A Recombinant Construct Encoding CIPK15

The present invention provides a recombinant construct encoding CIPK15. In particular, the recombinant construct comprises a nucleotide sequence encoding CIPK15 operably linked to a promoter.

As used herein, the term “recombinant construct” refers to a polynucleotide or nucleic acid having sequences that are not naturally joined together. A recombinant construct may be present in the form of a vector. “Vectors” may contain a given nucleotide sequence of interest and a regulatory sequence. Vectors may be used for expressing the given nucleotide sequence (expression vector) or maintaining the given nucleotide sequence for replicating it, manipulating it or transferring it between different locations (e.g., between different organisms). Vectors can be introduced into a suitable host cell for the above-mentioned purposes. A “recombinant cell” refers to a cell where a recombinant nucleic acid is introduced.

As used herein, the term “operably linked” may mean that a polynucleotide is linked to an expression control sequence in such a manner to enable expression of the polynucleotide when a proper molecule (such as a transcriptional factor) is bound to the expression control sequence.

As used herein, the term “expression control sequence” or “regulatory sequence” means a DNA sequence that regulates the expression of the operably linked nucleic acid sequence in a certain host cell.

Examples of vectors include, but are not limited to, plasmids, cosmids, phages, YACs or PACs. Typically, in vectors, the given nucleotide sequence is operatively linked to the regulatory sequence such that when the vectors are introduced into a host cell, the given nucleotide sequence can be expressed in the host cell under the control of the regulatory sequence. The regulatory sequence may comprise, for example and without limitation, a promoter sequence (e.g., the cytomegalovirus (CMV) promoter, simian virus 40 (SV40) early promoter, T7 promoter, and alcohol oxidase gene (AOX1) promoter), a start codon, a replication origin, enhancers, an operator sequence, a secretion signal sequence (e.g., □-mating factor signal) and other control sequence (e.g., Shine-Dalgano sequences and termination sequences). Preferably, vectors may further contain a marker sequence (e.g., an antibiotic resistant marker sequence) for the subsequent screening procedure. In vectors, the given nucleotide sequence of interest may be connected to another nucleotide sequence other than the above-mentioned regulatory sequence such that a fused polypeptide is produced and beneficial to the subsequent purification procedure. Said fused polypeptide includes, but is not limited to, a His-tag fused polypeptide and a GST fused polypeptide. The given nucleotide sequence of interest may also be connected to a reporter gene e.g. β-glucuronidase (GUS) or green fluorescent protein (GFP) to monitor promoter activity and gene expression.

Where the expression vector is constructed for a plant cell, several suitable promoters known in the art may be used, including but not limited to the Figwort mosaic virus 35S promoter, the cauliflower mosaic virus (CaMV) 35S promoter, the commelina yellow mottle virus promoter, the rice cytosolic triosephosphate isomerase (TPI) promoter, the rice actin 1 (Act1) gene promoter, the uniquitin (Ubi) promoter, the rice amylase gene promoter, the adenine phosphoribosyltransferase (APRT) promoter of Arabidopsis, the mannopine synthase and octopine synthase promoters.

In some embodiments, a promoter described herein may be heterologous to the nucleic acid encoding a target gene in the vector. As used herein, a promoter heterologous to a coding sequence (a gene) refers to a promoter that is not the natural promoter that controls (drives) expression of the gene in native state. For example, the vector of the present disclosure may comprise a promoter derived from a non-CIPK15 gene.

In some examples, the promoter described herein can be constitutive, which initiates transcription independent of the influence of regulation. Exemplary constitutive promoters include, but are not limited to a maize ubiquitin (Ubi) promoter, a rice actin (Act1) promoter, and a cauliflower mosaic virus 35S (CaMV35S) promoter.

In other examples, the promoter described herein can be inducible, which initiates transcription in a regulated manner, for example, in the presence or absence of a particular factor. Exemplary inducible promoters include an ethanol inducible promoter (e.g., a A1cR/A1cA promoter) or a β-estradiol inducible promoter (e.g., a XVE promoter, see Examples section below). Exemplary promoters inducible by biotic or abiotic stress (e.g., osmotic stress, drought stress, salt stress, high or low temperatures, hypoxia, anoxia, hydration, pH, chemicals, hormones or a combination thereof) include an Arabidopsis rd29A promoter, an Arabidopsis corl SA promoter, an Arabidopsis kinl promoter, an Arabidopsis heat-shock factor (HSF) promoter, and an alpha-amylase promoter.

In some embodiments, the promoter described herein may be homologous to the nucleic acid encoding a target gene in the vector. As used herein, a promoter homologous to a coding sequence (a gene) refers to a promoter that is the natural promoter that controls (drives) expression of the gene in native state. For example, the vector of the present disclosure may comprise a promoter derived from a native CIPK15 gene (a native CIPK15 promoter).

In certain example, a promoter sequence as used in the invention is a Ubi promoter having SEQ ID NO: 34.

In certain example, a promoter sequence as used in the invention is a XVE promoter having SEQ ID NO: 35.

In certain example, a promoter sequence as used in the invention is a native rice CIPK15 promoter having SEQ ID NO: 36.

In some embodiments, a vector comprising a nucleic acid encoding CIPK15 operably linked to a Ubi promoter comprises a fused promoter/coding region fragment of SEQ ID NO: 37 (Ubi: CIPK15).

In some embodiments, a vector comprising a nucleic acid encoding CIPK15 operably linked to a XVE promoter comprises a fused promoter/coding region fragment of SEQ ID NO: 38 (XVE: CIPK15).

In some embodiments, a vector comprising a nucleic acid encoding CIPK15 operably linked to a CIPK15 promoter comprises a fused promoter/coding region fragment of SEQ ID NO: 39 (CIPK15: CIPK15).

A recombinant construct described herein may be prepared via conventional recombinant technology.

### III. Host Cells, Transgenic Plants, and Methods for Making them

Some aspects of the present invention provide host cells (e.g., an Agrobacterium cell or a plant cell) comprising any of the vectors as described herein. These host cells (or called recombinant cells) carry exogenous/foreign genetic materials (e.g., the vectors described herein), which can be introduced into the host cell via conventional practice. “Exogenous genetic materials” as used herein can mean that the genetic materials are not originally present in the cells and instead artificially introduced into the cells of a parent thereof. In some instances, the exogenous genetic material may be derived from a different species as the host cell. In some other instances, the exogenous genetic material may be derived from the same species as the host cell and introduced into the host cell such that the resultant recombinant cell comprises extra copies of the genetic material as compared with the wild-type counterpart. The term “transformation” or “transform” as used herein refers to the introduction of exogenous genetic materials into a host cell such as a plant cell.

In certain embodiments, the host cell may be an Agrobacterium host cell. In certain embodiments, the host cell may be a plant cell, for example, a cell from a monocotyledonous plant or a dicotyledonous plant.

Suitable conventional methods are available to make the recombinant cells described herein. Examples of such methods include electroporation, PEG operation, particle bombardment, micro injection of plant cell protoplasts or embryogenic callus or other plant tissue, or Agrobacterium-mediated transformation.

Transgene expression (e.g., before and after transformation of a vector presented herein in a host cell) may be detected using methods known in the art. For example, reverse transcriptional polymerase chain reaction (RT-PCR) may be used to determine mRNA expression. Additional detection methods include western blot analysis and an enzyme-linked immunosorbent assay (ELISA) with a proper antibody for protein detection.

A transgenic plant can be grown from a recombinant cell. Therefore, the present invention provides a transgenic plant comprising an exeogenous nucleic acid operably linked to a promoter, wherein the exeogenous nucleic acid (a transgene) encodes CIPK15.

As used herein, plants may be a full plant or a part thereof, including a fruit, shoot, stem, root, leaf or seed, or various types of cells in culture (e.g., single cells, protoplasts, embryos, callus, protocorm-like bodies, and other types of cells). As described above, a plant of the present disclosure may be a monocot or a dicot.

In some embodiments, the plants as described herein are monocotyledonous plants. Examples of monocots include, but are not limited to, rice, barley, wheat, rye, oat, corn, bamboo, sugarcane, onion, leek, ginger, stiff brome, millet, green foxtail, sorghum, cutgrasses, Kans grass or Hall's panicgrass.

In other embodiments, the plants described herein are dicotyledonous plants. Exemplary dicot plants include Arabidopsis, soybean, peanut, sunflower, safflower, cotton, tobacco, tomato, pea, chickpea, pigeon pea and potato.

A variety of procedures that can be used to engineer a stable transgenic plant are available in this art. In some embodiments, the transgenic plant is produced by transforming a tissue of a plant, such as a protoplast or leaf-disc of the plant, with a recombinant Agrobacterium cell comprising a nucleic acid encoding a desired protein (e.g. CIPK15) and generating a whole plant from the transformed plant tissue. In some embodiments, a nucleic acid encoding a desired protein can be introduced into a plant via gene gun technology, particularly if transformation with a recombinant Agrobacterium cell is not efficient in the plant. To prepare a transgenic plant, it is preferably that the expression vector as used herein carries one or more selection markers for selection of the transformed plants, for example, genes conferring the resistance to antibiotics such as hygromycin, ampicillin, gentamycine, chloramphenicol, streptomycin, kanamycin, neomycin, geneticin and tetracycline, URA3 gene, genes conferring the resistance to any other toxic compound such as certain metal ions or herbicide, such as glufosinate or bialaphos.

Specifically, a transgenic plant according to the present invention comprises a transgene encoding CIPK15 allowing for overexpression of CIPK15 in the transgenic plant. For example, the level of CIPK15 in the transgenic plant may be at least 10% higher (e.g., 20% higher, 30% higher, 50% higher, 1-fold higher, 2-fold higher, 5-folder higher, 10-fold higher, or above) as compared with that in non-transgenic (wild type) counterpart plants. In some instances, the wild-type parent does not express the such CIPK15.

According to the present invention, a transgenic plant as disclosed herein exhibits improved growth, yield and/or root development. For example, a transgenic plant as disclosed herein exhibits increased plant height, elongated coleoptiles and shoot, higher grain yield, longer roots, larger root radius, and/or more aerenchyma, compared to a non-transgenic (wild type) counterpart plant, under the same growing conditions. In some embodiments, the growing condition is aerobic or hypoxic.

In some embodiments, a transgenic plant as disclosed herein further exhibits enhanced tolerance to environmental stress. For example, a transgenic plant as disclosed herein exhibits improved ability to survive under environmental stress. In some instances, the environmental stress includes drought, salinity and/or flooding stresses.

In some embodiments, a transgenic plant as disclosed herein further exhibits enhanced ethylene production.

Accordingly, the present invention also provides a method of producing the transgenic plants described herein. The method may comprise: (a) transforming plant cells with a recombinant construct comprising a nucleic acid operably linked to a promoter to obtain recombinant plant cells overexpressing CIPK15, wherein the nucleic acid encodes CIPK15; and (b) growing the recombinant plant cells obtained in (a) to generate the transgenic plants.

The present invention further provides a method for improving growth, yield and/or root development of a plant by overexpressing CIPK15 in the plant. In particular, the method comprises (a) transforming plant cells with a recombinant construct comprising a nucleic acid operably linked to a promoter to obtain recombinant plant cells overexpressing CIPK15, wherein the nucleic acid encodes CIPK15; (b) growing the recombinant plant cells obtained in (a) to generate a plurality of transgenic plants; and (c) selecting a transgenic plant from the plurality of transgenic plants generated in (b) that exhibits improved growth, yield and/or root development as compared with a non-transgenic plant counterpart growing under the same conditions. In some embodiments, the transgenic plant as selected exhibits improved growth, yield and/or root development as compared with a non-transgenic plant counterpart under aerobic or hypoxic condition. In some examples, the improved growth, yield and/or root development may include increased plant height, elongated coleoptiles and shoot, higher grain yield, longer roots, larger root radius, and/or more aerenchyma. In some embodiments, the transgenic plant as selected further exhibits enhanced tolerance to environmental stress, such as drought, salinity and/or flooding stresses.

Genome/gene editing methods are also available to introduce targeted mutation in plants. Typically, targeted gene editing may include deletion, insertion and/or substitution at one or more given sites in the genome to give mutation to genes in the plant cells. Examples of gene editing methods include zinc finger nuclease (ZFN), transcription activator-like effector DNA binding protein (TALLEN) and clustered regularly interspaced short palindromic repeats (CRISPR)/Cas system. Typically, in CRISPR/Cas system, the key components include a Cas protein (endonuclease) and a guide RNA (gRNA) containing a 20-nt guide sequence that can direct the Cas protein to the target site. The Cas protein can be a Cas9 enzyme that may be one from Streptococcus pyogenes while other Cas9 homologs may also be used. The Cas protein creates a DNA double-strand break (DSB) in the genome.

During the DSB repair, gene mutagenesis or replacement can be obtained via non-homologous end joining leading to nucleotide deletion, insertions and substitution around the DSB region and disrupting gene function, or by homologous recombination to provide precise gene editing in the presence of a repair template. In some embodiments, a binary vector is designed and used which expresses two elements, a Cas9 protein and a gRNAs with a target sequence e.g. CIPK15 (V48I), to cleave target genomic regions. Delivery of a Cas protein and a gRNA may be through direct injection or cell transfection using known methods, for example, Agrobacterium, gene gun, protoplast transfection and viral delivery. In some examples, a Cas protein and a gRNA or a binary vector expressing both are/is introduced into plants through Agrobacterium-mediated transformation procedures and a gene-edited plant is regenerated from the transformed plant cells. In some examples, a Cas protein and a gRNA or a binary vector expressing both are/is introduced into an isolated plant protoplast and a gene-edited plant is regenerated from the transfected plant protoplast.

In some embodiments, a gene-edited plant is thus provided which comprises a genome comprising a nucleic acid encoding a CIPK15 having an isoleucine (Ile) substituent at a position corresponding to position 48 of SEQ ID NO: 1. In particular, the plant, before genome edition, originally has a natural gene encoding a CIPK15 having an amino acid residue other than isoleucine (Ile) at a position corresponding to position 48 of SEQ ID NO: 1 in its genome, and after genome edition, the gene is modified to encode a mutant CIPK15 having an isoleucine (Ile) substituent at the position corresponding to position 48 of SEQ ID NO: 1 in its genome. In some examples, the amino acid residue other than isoleucine (Ile) at a position corresponding to position 48 of SEQ ID NO: 1 includes valine (Val) or alanine (Ala).

In some embodiments, the mutant CIPK15 comprises (a) an amino acid sequence selected from the group consisting of SEQ ID NOs:19-32; or (b) an amino acid sequence having a sequence identity of at least 80% with the amino acid sequence of (a) and having an isoleucine (Ile) residue at position 48 of SEQ ID NO: 1.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### Examples

In this study, we show that protein kinase CIPK15 transmits low oxygen signal to activate genes essential for sugar and energy generation, ethylene and auxin biosynthesis, and root development under hypoxia. We also demonstrate HXK5/6-VDAC1 complex interacting with and inactivating CIPK15 on mitochondria in a sugar- and oxygen-dependent manner, revealing the structure-function relationship of CIPK15-HXK6-VDAC1 interaction. Under hypoxia, CIPK15 expression, phosphorylation and stability are enhanced, whereas expression of HXK5/6 and VDAC1 is reduced, thus enabling sufficient CIPK15 functionality to induce genes necessary for underwater root growth. Moreover, root-specific induction of CIPK15 prompted a 20-50% increase in grain yield in a hypoxic rice paddy. Our study reveals a unique mitochondria-based machinery in rice for sensing oxygen and promoting growth under hypoxia.

### 1. Material and Methods

### 1.1 Plant Materials and Growth Conditions

Rice seeds were sterilized in 3% (v/v) sodium hypochlorite for 1 h and washed with sterile deionized water. Seeds were laid on 1/2X MS agar (0.3% CultureGel™ Type1, Phytotechlab) medium without sucrose in 400 ml bottles filled with or without deionized water for submerged or aerobic treatments, respectively. Seeds were germinated at 28° C. under a 16:8 h light:dark cycle in a growth chamber. Water for submergence treatment was de-gassed with a vacuum pump and flushed with nitrogen before use. To examine root morphology, seeds were pre-germinated on 1/2X MS agar plates at 37° C. in the dark for 2 days, before transferring the seedlings to glass tubes (25 cm in height, 5 cm in diameter), with 5 seedlings per tube, for aerobic or submerged treatments in the growth chamber for another 12 days.

Setaria viridis seeds were sterilized in 0.6% (v/v) sodium hypochlorite plus 0.1% tween-20 for 10 minutes and washed with sterile deionized water. Seeds were laid on 1/2X MS agar medium without sucrose in glass jars and incubate at 4° C. for 2 weeks to break the dormancy, then the jars were shifted to a growth chamber setting at 24° C. under a 16:8 h light:dark cycle.

### 1.2 Plasmid Construction

For inducible overexpression of CIPK15, full-length CIPK15 cDNA was inserted into pXVE:dHA vector between NotI and SpeI restriction sites, and then the plasmid was further inserted between KpnI and SpeI sites in pCAMBIA1301, generating pXVE:CIPK15-dHA. For CIPK15 promoter-driven GUS expression, the 2.0-kb CIPK15 promoter region plus the 1.4-kb 5′ untranslated region (UTR), including the first intron, was inserted into the pENTR™/D-TOPO™ vector (Invitrogen), generating pENTR-pCIPK15, and this plasmid was inserted into the binary vector pccdB:GUS/pSMY1H using the Gateway® LR system, generating pCIPK15:GUS. For LAX promoter-driven GUS expression, 2037-, 2629- or 2246-bp upstream of the ATG translation start codon, were inserted between EcoRI and NcoI sites of pCAMBIA1305.1, generating pLAX1:GUS, pLAX3:GUS and pLAX5:GUS, respectively.

To construct CIPK15:CIPK15, the CIPK15 promoter from pENTR-pCIPK15 was amplified with KpnI and NcoI restriction sites, and the coding region of CIPK15 was amplified with NcoI and SpeI restriction sites. The pCAMBIA1305.1 binary vector was then digested with KpnI and SpeI, allowing us to ligate the CIPK15 promoter and coding region, thereby generating the CIPK15:CIPK15 construct.

For plasmids used in our protoplast transient expression assays, cDNAs of CIPK15, HXK5, HXK6 and VDAC1 were inserted into pENTR vectors and then inserted into pUbi:ccdB-mCherry, pUbi:ccdB-GFP, p35S:cYFP-ccdB, p35S:ccdB-nYFP or pUbi:ccdB-HA binary vectors via the Gateway LR system, thereby generating CIPK15, HXK5 and HXK6 fused to desirable reporter genes.

The CIPK15 and HXK6 mutants were generated using pENTR-CIPK15 and pENTR-HXK6 as templates to construct different CIPK15 (ΔNAF, ΔPPi, T₁₇₁D, T₁₇₁A, NAF₃₁₅₋₃₁₇PPP, L₃₂₈P/F₃₃₂P) and HXK6 (ΔTS, G₁₁₂D, K₂₀₃A) entry vectors by polymerase chain reaction (PCR) with specific primers and annealing with blunt-end ligation. pENTR:CIPK15ΔPPi was used as template for generating pENTR:CIPK15ΔPPi/Ti₇₁D and pENTR:CIPK15ΔPPi/T₁₇₁A. These entry vectors were inserted into pUbi:ccdB-mCherry or pUbi:ccdB-GFP binary vectors using the Gateway LR system.

For RNAi construction, the 3′ UTRs of HXK5 and HXK6 were used to generate pENTR-HXK5/6-3′UTR vectors, which were inserted into the pUbi:ccdB-GFPccdB-NosT vector via the Gateway LR system. The pUBQ10:CIPK15-mTagBFP construct was generated by amplifying CIPK15 cDNA and ligating it into the pGGM000 binary vector according to the instructions of the Greengate cloning system³⁰.

### 1.3 Plant Anatomy and Microscopy

To analyze aerenchyma area, seminal roots of 14-day-old seedlings were cross-sectioned at different positions from the root tip using a Microslicer™ (DTK-1000) vibrotome. Sections were stained with toluidine blue (Sigma-Aldrich) before capturing images under a microscope (Axioimager Z1, Carl Zeiss). The area of aerenchyma was determined in ImageJ software (Ver. 1.8.0, US National Institutes of Health).

### 1.4 RNA-Seq and Bioinformatics Analyses

Total mRNA was extracted from 5-day-old seedlings using the RNeasy Plant Mini Kit (Qiagen). RNA quality control and quantification were carried out using a Bioanalyzer 2100 system (Aligent). cDNA libraries were prepared with a TruSeq standard mRNA kit (Illumina) according to the manufacturer's instructions. cDNA sequencing was performed using a 500-high output v2 sequencing kit and an Illumina Nextseq500 instrument. Bioinformatics analysis was conducted in CLC Genomics Workbench software (v11.0.1, Qiagen).

### 1.5 Real-Time Quantitative RT-PCR Analysis

Total RNA was extracted from roots of 14-day-old seedlings using Trizol reagent (Invitrogen) and treated with DNase I (Thermo Scientific) at 37° C. for 15 min. cDNA was synthesized from 1 μg of RNA using the Promega Reverse Transcription system and diluted 5-fold for further use. Real-time qRT-PCR was performed as described previously³¹.

### 1.6 Luciferase and GUS Activity Assays and GUS Staining

Luciferase and GUS activity in transfected rice embryo calli were assessed as described previously³². GUS staining of rice seedlings was conducted as described previously³³.

### 1.7 Rice Transformation

Plasmids pCIPK15:GUS and pXVE:CIPK15-dHA, as well as CIPK15:CIPK15, were introduced into Agrobacterium tumefaciens strain EHA105 and then rice transformation was performed as described previously 34. Plasmids OsCIPK15:OsCIPK15, were introduced into Agrobacterium tumefaciens strain EHA105 and then S. vridis transformation was performed as followed₃₄.

### 1.8 Rice Protoplast Transient Expression Assay

Stems of 10-day-old rice seedlings were used to isolate protoplasts as described previously³⁵. After transfection, protoplasts were cultured in 2 ml culturing medium in 6-well plates (Jet-Biofil) for 4 h on an orbital shaker at 60 rpm, and then divided into aerobic or submerged treatment groups. For aerobic treatment, protoplasts were kept under shaking in a 6-well plate. For submerged treatment, protoplasts in a 6-well plate were placed on the bench without shaking to keep the medium stagnant. All protoplasts were incubated at 25° C. in the dark for 12 h.

### 1.9 Subcellular Localization Analysis of Reporter-Fused Proteins

Fluorescence images were collected with a Zeiss LSM 780 confocal microscope. BFP, GFP/YFP and mCherry fluorescence images were detected with the laser line of excitation/emission at 405/415-470, 488/505-540 and 561/594-638 nm, respectively. Mitochondria were stained using Mitotracker™ Green FM (Thermo) for 15 min according to the manufacturer's instructions prior to examination under confocal microscopy.

### 1.10 Antibodies and Immunoblot Analysis

Sources of antibodies: anti-HA antibody from Proteintech (66006-2-Ig), anti-tubulin antibody from Agrisera (AS20-4483), anti-GFP antibodies from GeneTex (GTX113617), anti-RFP antibody from ChromoTek (6G6), anti-GUS antibody from Sigma-Aldrich (G5420), and anti-CIPK15 antibody from Abbexa (abx018654). All antibodies were diluted 5000-fold before use. We conducted immunoblotting as described previously³².

### 1.11 Immunoprecipitation

Samples were extracted from rice protoplasts in lysis buffer containing 150 mM NaCl, 0.5% NP4-0, 20 mM Tris-HCl (pH 7.5), and protease inhibitor (Roche) for 1 h at 4° C. and centrifuged at 13,000 rpm for 30 min at 4° C. Supernatants were transferred to new tubes and GFP- or RFP-conjugated proteins were pulled down with GFP-Trap®_MA beads or RFP-Trap®_MA beads. Precipitated proteins were subjected to immunoblot analysis using anti-RFP and anti-GFP antibodies.

### 1.12 Immunoprecipitation (IP)—Mass Spectrometry

Two-day-old seedlings of the XVE: CIPK15-dHA and XVE:GFP-dHA (serving as control) transgenic lines were transplanted into 1/2X MS medium containing 5 □M β-estradiol for 5 days under aerobic or submerged conditions. Total proteins were extracted in lysis buffer containing 25 mM Tris-HCl (pH 7.5), 150 mM NaCl, 1% NP-40, 1 mM EDTA, 5% glycerol and protease inhibitor (Roche) and subjected to IP using Pierce™ Anti-HA Magnetic Beads to pull down HA-conjugated proteins. Eluted proteins were digested with trypsin before undergoing LTQ mass spectrometry-based proteomic analysis. CIPK15-interacting proteins were identified from XVE:CIPK15-dHA transgenic lines, but not from the control, under highly stringent criteria with a false discovery rate (FDR) of <1% for peptide identification. The peptide fragmentation data from LC-MS/MS were searched against RAP-DB (https://rapdb.dna.affrc.go.jp/) using MASCOT software (Matrix Science Ltd.).

### 1.13 Phospho-Peptide Mapping and Protein Phosphorylation Analysis

Total proteins extracted from roots of the XVE:CIPK15-dHA transgenic line for IP-MS (described above) were used for phosphor-peptide mapping, as described previously³¹

### 1.14 Field Test

To evaluate grain weight per plant in the field, 25-day-old seedlings were transplanted into soil, with 25×25 cm spacing between each plant, with a total of 10 plants of each line grown in the field and 15 plants of each line grown as one plot. Seeds were harvested after ripening, dried, and their weight per plant was determined as described previously³³.

### 1.15 Ethylene Treatment

Five-day-old seedlings were treated with 2 or 5 mM of the ethylene precursor 1-aminocyclopropane-1-carboxylic acid (ACC) (Sigma) or [[concentration??]] the ACS inhibitor aminoethoxyvinylglycine (AVG) (Sigma) for 1 day.

### 1.16 Lignin and Suberin Staining

Seedlings were grown under aerobic or hypoxic conditions for 7 days. Seminal roots were embedded in 5% agar, and positions at 1.5 cm above root tips were cross-sectioned at a 100-μm thickness using a vibratome Microslicer™ (DTK-1000) Vibrotome. Root sections were fixed with methanol. To examine lignin distribution, sections were stained with acriflavine as described previously (54) and imaged with a Zeiss LSM 780 confocal microscope using a 488 nm laser line for excitation and a 505 to 550 nm band pass filter for emission. To examine suberin lamellae, sections were stained with fluorol yellow 088 as described previously (55, 56) and imaged with a Zeiss LSM 780 confocal microscope using an excitation wavelength of 488 nm and 524 to 594 nm for emission.

### 1.17 Statistical Analyses

Luciferase activity in protoplasts transfected with the reporter construct alone was set to 1, and other values in protoplasts transfected with both effector and reporter constructs were calculated relative to this value (relative values are shown above bars in bar plots). Error bars indicate the SE or SD from three independent experiments. *, ** and *** represent significance levels of P<0.05, 0.01 and 0.001, respectively, according to Student's t-test. n.s. indicates no significant difference. For bar plots displaying letters above the bars, statistical analysis was performed by one-way ANOVA with a post-hoc Tukey HSD. Significant levels are labeled at p<0.05.

### 2. Results

### 2.1 CIPK15 Regulates Aerenchyma and Root Development

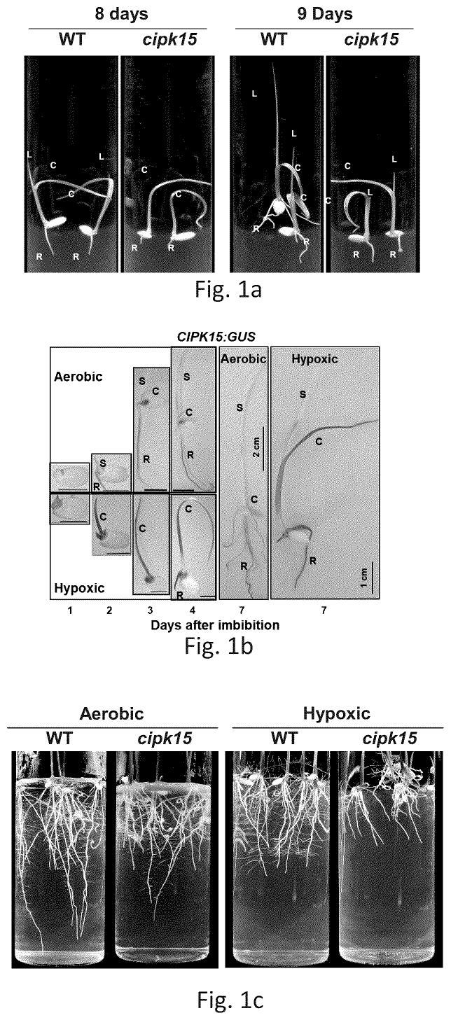

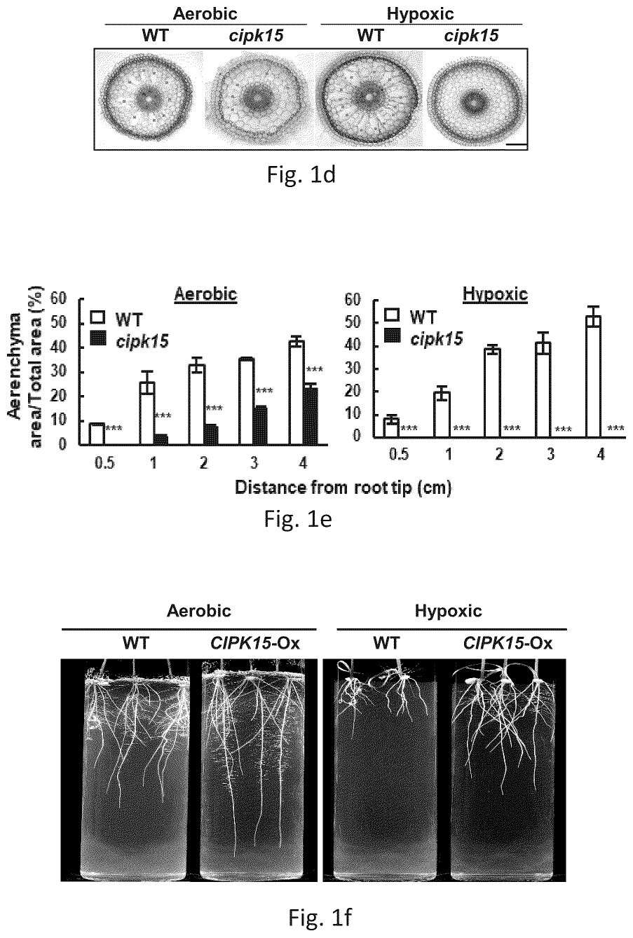

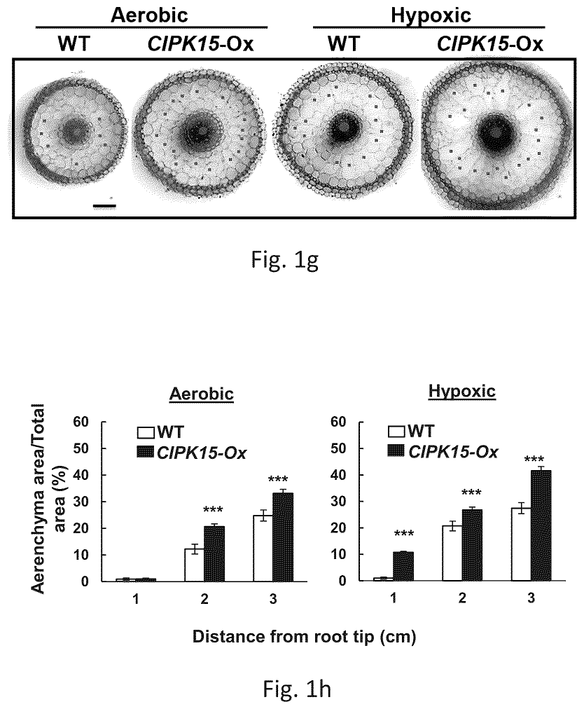

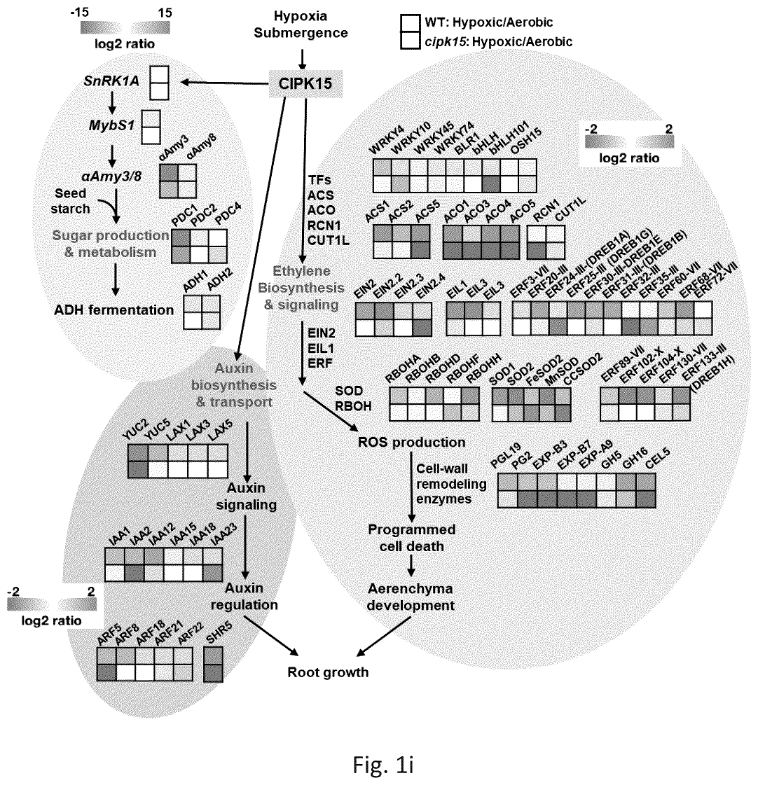

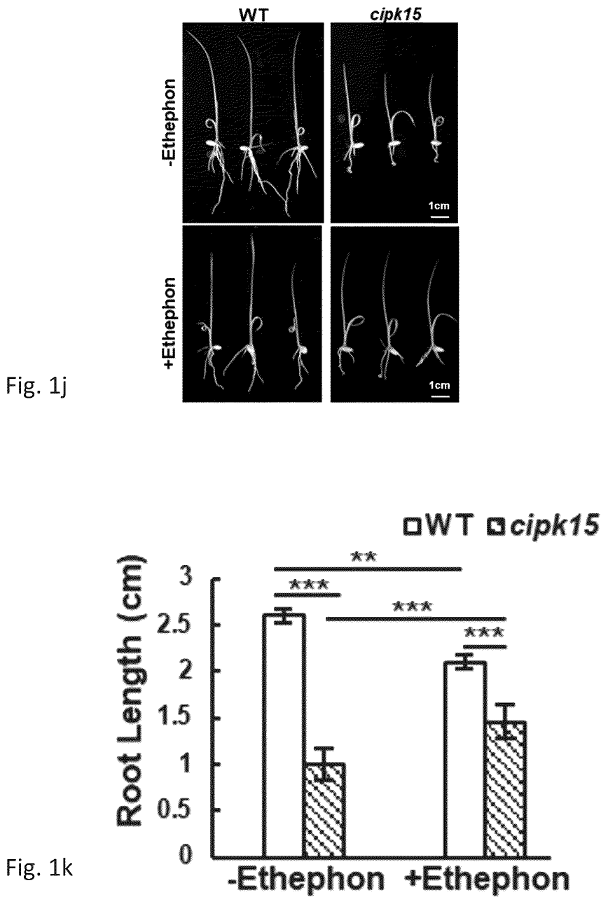

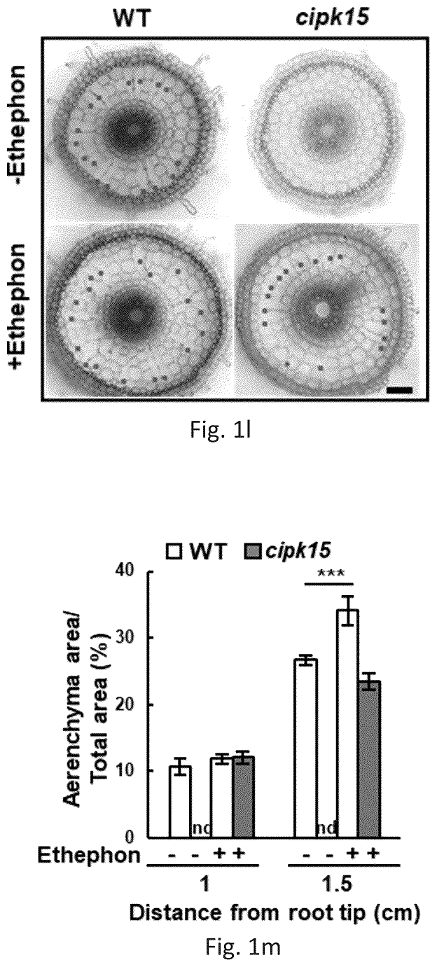

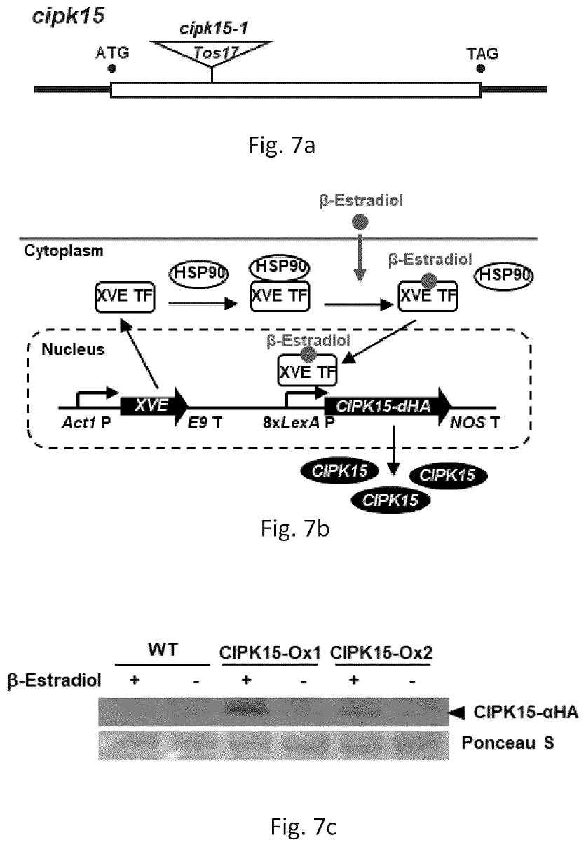

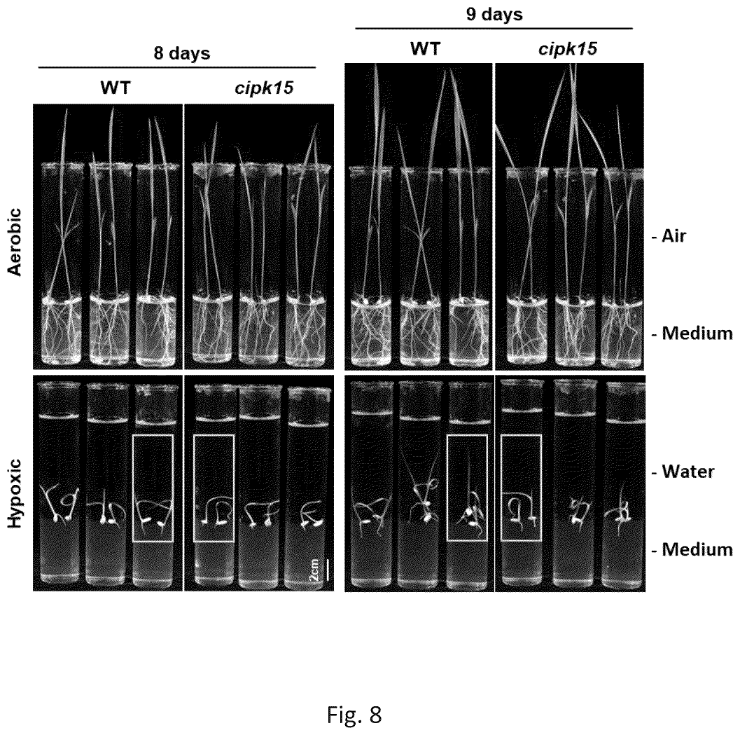

We grew seedlings of wild type (WT) (cv. Nipponbare) and cipk15 knockout mutant plants (FIG. 7a) in agar medium, with the shoot part either exposed in air (aerobic) or submerged in water (hypoxic). For 8-9-day-old seedlings, the leaves and roots of the cipk15 mutant grew slightly more slowly under aerobic conditions compared to WT, but significantly more slowly under hypoxia (FIG. 1a and FIG. 8). We generated CIPK15: GUS transgenic lines and found that the CIPK15 promoter is active mainly in embryos, coleoptiles and roots, but not in leaves, and its activity is enhanced under hypoxia (FIG. 1b). Given that coleoptiles soon undergo PCD to facilitate leaf development¹⁴, CIPK15 seems to play a more important role in root development.

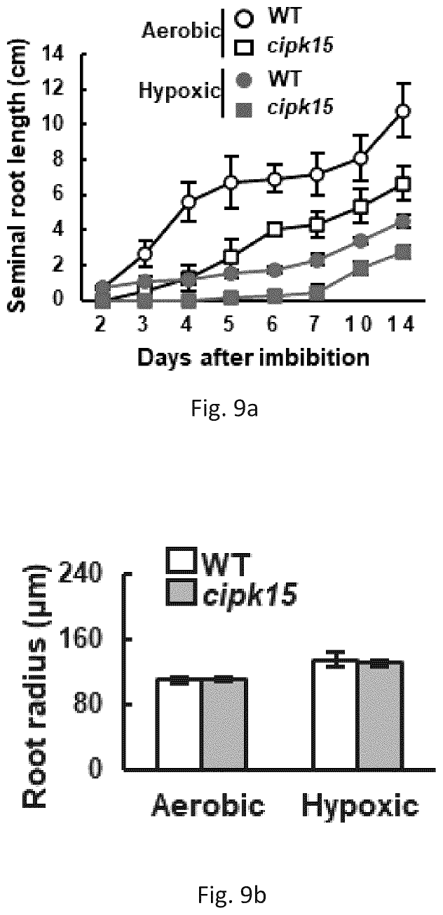

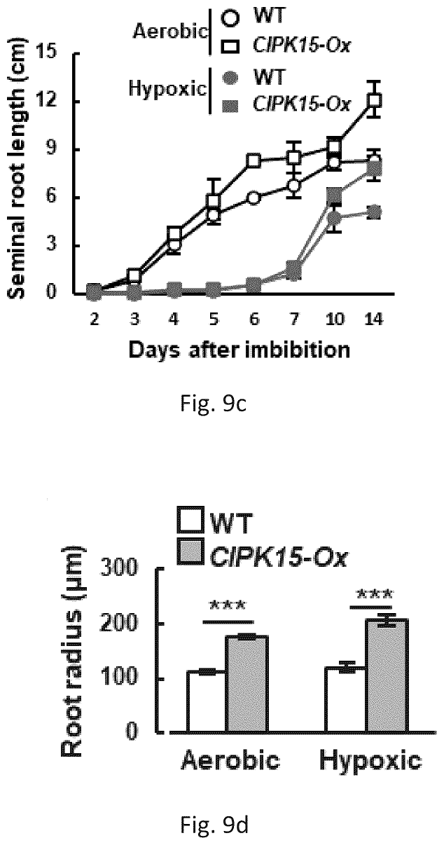

Aerenchyma development facilitates vertical O₂ transmission and enhances hypoxia tolerance, and it is an important indicator of active root growth¹⁵. We observed that aerenchyma and root development were retarded in 14-day-old cipk15 seedlings maintained under aerobic conditions and they were almost completely inhibited under hypoxia compared to WT (FIGS. 1c to 1e and FIG. 9a). Overexpression of CIPK15 under the control of the strong constitutive Ubi promoter severely inhibits plant growth of transgenic rice (cv. Tainung 67). Consequently, we overexpressed CIPK15 under the control of a β-estradiol-inducible XVE promoter³⁶ in transgenic rice (FIG. 7b, FIG. 7c). We observed that root elongation, aerenchyma development, and root radius were all enhanced in the CIPK15-overexpressing lines compared to WT under both aerobic and hypoxic conditions (FIGS. 1f to 1h and FIG. 9c, FIG. 9d). Moreover, aerenchyma development at 1 cm from the root tip was barely detectable in the WT, but it was significantly enhanced in the CIPK15-overexpressing line under hypoxia (FIG. 1h).

### 2.2 CIPK15 Regulates Aerenchyma and Root Development Under Hypoxia Through Ethylene- and Auxin-Dependent Pathways

We performed an RNA sequencing (RNA-seq) analysis on WT and cipk15 mutant lines to identify genes regulated by CIPK15 under hypoxia. Apart from genes known to be involved in sugar production and metabolism in post-germination seedlings, we uncovered many genes involved in ethylene biosynthesis and signaling and aerenchyma development that were downregulated in the cipk15 line (FIG. 1i). More specifically, these genes included ACS and ACO for ethylene biosynthesis and EIN2, EIN3/EIL1 and ERFs for ethylene signaling, RBOHs for ROS production, PGs, EXPs, CELs and GHs for cell wall degradation, and PCD active in aerenchyma development of cortical cells. We also detected that genes regulating root development, including YUCs and LAXs for auxin biosynthesis and transport, and IAAs, ARF and SHR for auxin signaling and regulation, were all downregulated in the cipk15 line.

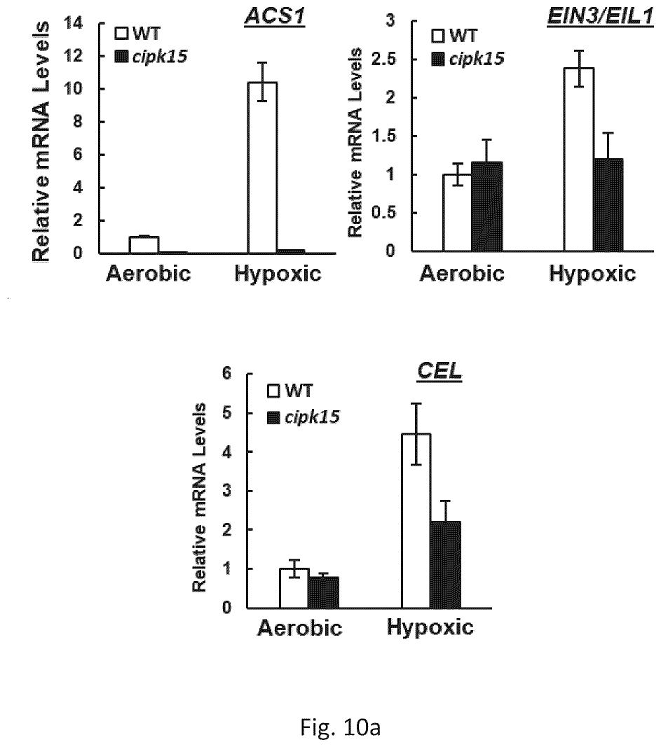

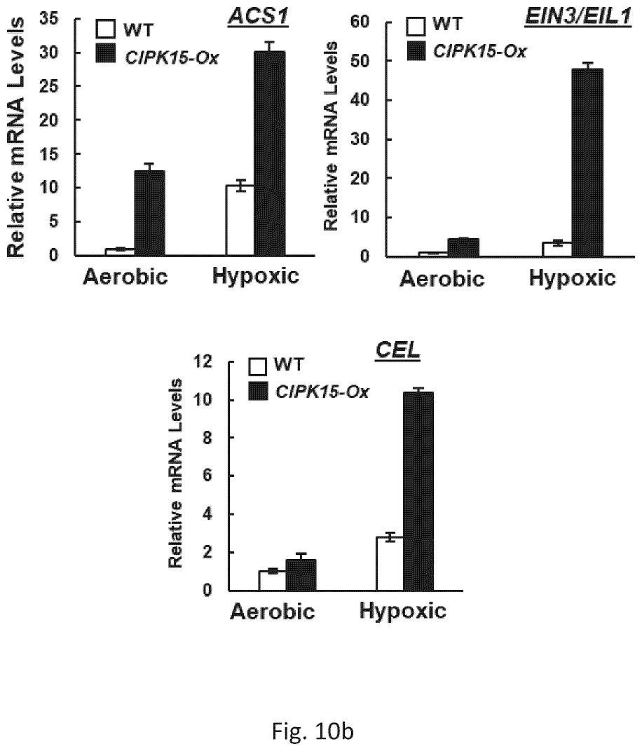

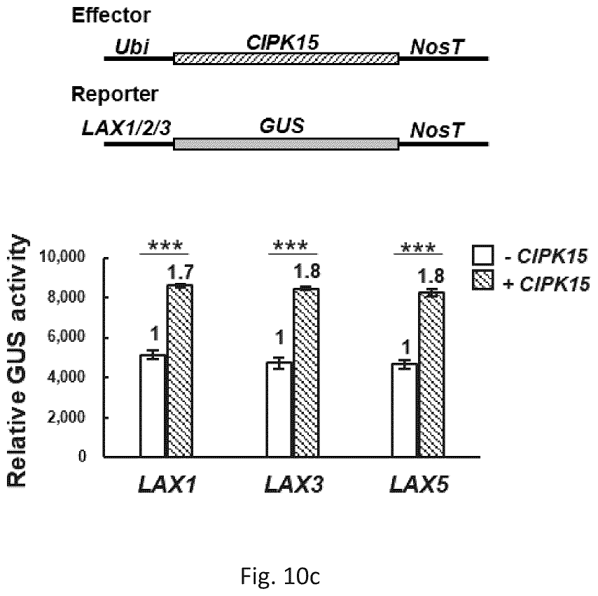

qRT-PCR analysis validated that levels of the ACS1, EIN3/EIN1 and CEL mRNAs were all increased in WT under hypoxia, but they were increased to a lesser extent under hypoxia in cipk15 (FIG. 10a). In contrast, mRNAs of these genes were all increased in the CIPK15-overexpressing line compared to WT under both aerobic and hypoxic conditions (FIG. 10b). We also analyzed promoters of LAX1/3/5 fused to the reporter gene GUS in transient expression assays, which revealed that the activity of all three LAX promoters was induced by CIPK15 (FIG. 10c).

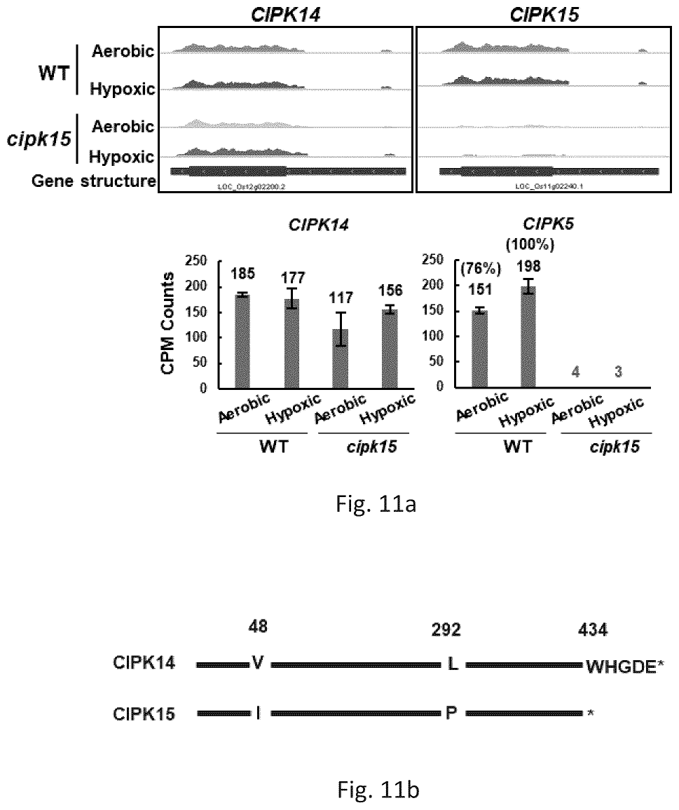

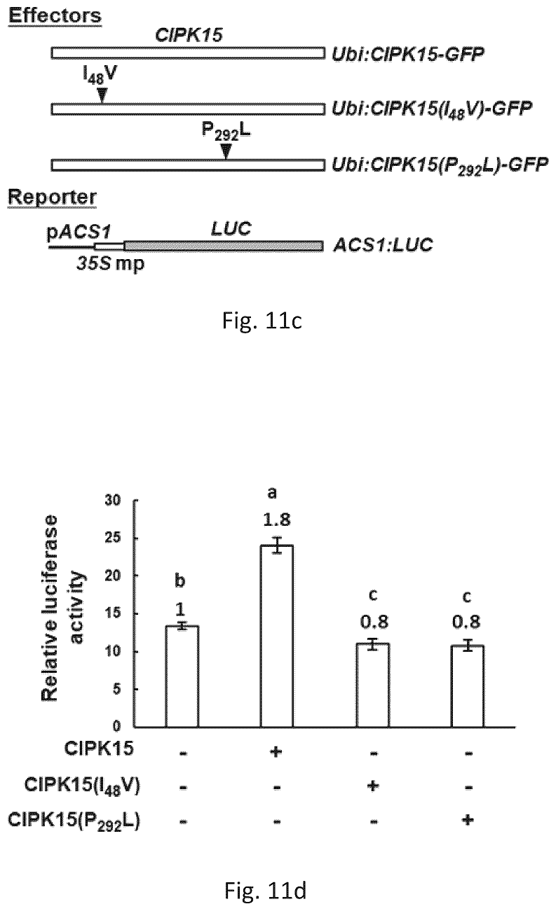

To confirm that CIPK15 regulates aerenchyma and root development through the ethylene-dependent pathway, rice seedlings were cultured in medium with or without ethephon under submergence. Ethephon can be converted to ethylene upon dissolving in water. We found that cipk15 seedlings grew more slowly than WT without ethephon, but faster with ethephon treatment (FIG. 1j). Without ethephon, roots were much longer in WT seedlings than in cipk15, whereas WT root growth was suppressed with ethephon that actually enhanced root growth in cipk15 (FIG. 1k). Aerenchyma development in cipk15 was also induced by ethephon (FIG. 11, FIG. 1m). High ethylene levels are known to inhibit rice root growth³⁷. Hence, the root growth suppressed by ethephon treatment in WT and enhanced by it in cipk15 could be attributable to higher and lower levels, respectively, of ethylene in the WT and cipk15 seedlings.

### 2.3 Sugar Starvation and Hypoxia Induce CIPK15 to Promote Seedling and Root Development and Grain Yield and the Amino Acid Ile 48 is Essential for CIPK15 Activity

CIPK14 and CIPK15 in the rice genome are highly homologous, with only a few nucleotide and amino acid differences in the cDNA and protein sequences, respectively, so qRT-PCR-based differentiation of the expression of these two genes is problematic. An RNA-seq analysis indicated that transcript levels are similar for CIPK14 and CIPK15, with only the CIPK15 transcript being abolished in the cipk15 line under both aerobic and hypoxic conditions (FIG. 11a). We substituted amino acid Ile 48 to Val (I₄₈V) and Pro 292 to Leu (P₂₉₂L) in CIPK15 (FIG. 11b), so that the two amino acids are identical in the mutated CIPK15 and WT CIPK14 proteins. A rice protoplast transient expression assay using WT or mutant CIPK15 as an effector and ACS1:LUC as a reporter revealed that WT CIPK15 could activate the ACS1 promoter, but the mutant CIPK15 (CIPK14-like) could not (FIG. 11c, FIG. 11d). This result indicates that CIPK14 is probably non-functional or exerts a very limited role in regulating ethylene biosynthesis. This result also indicates that the amino acid Ile 48 is essential for CIPK15 activity.

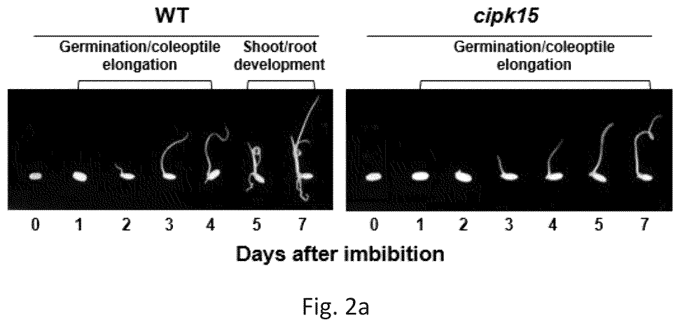

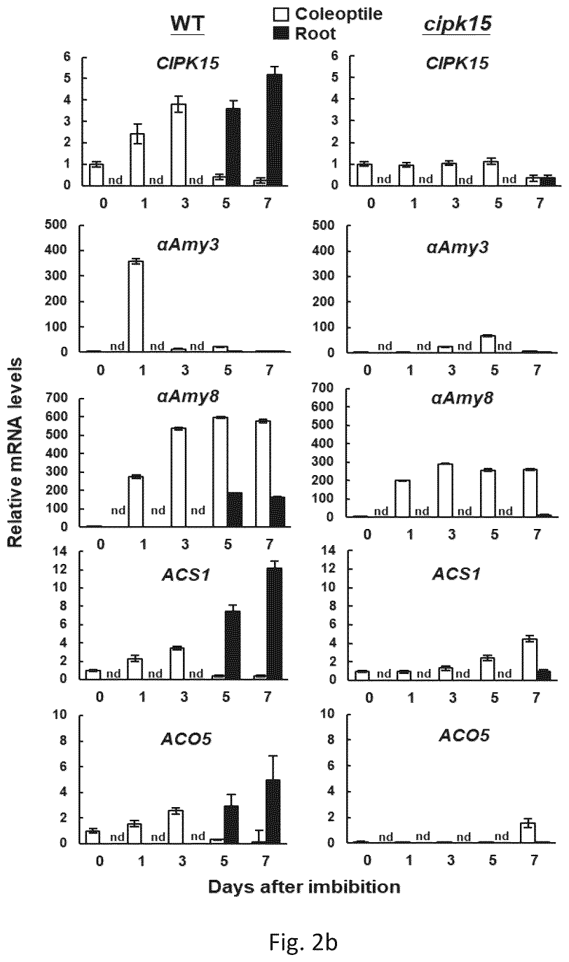

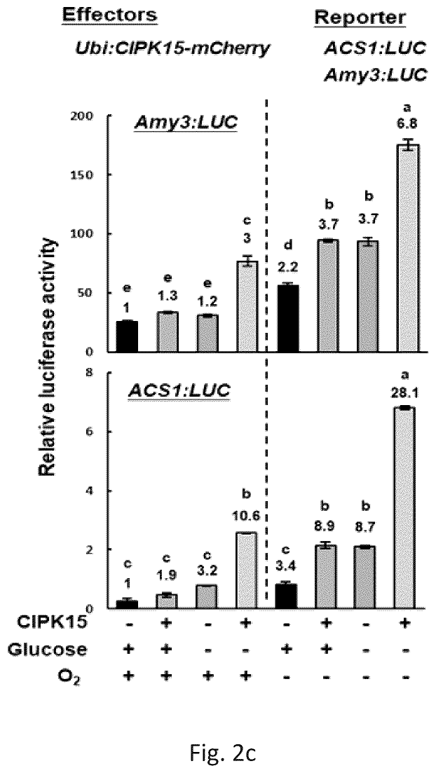

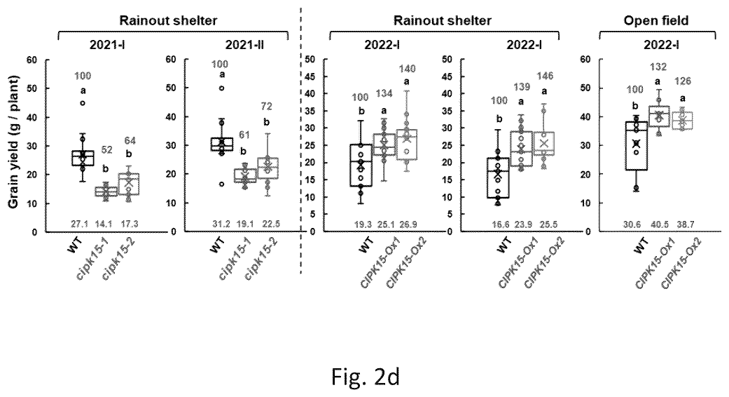

Under hypoxia, post-germination seedling development can be divided into two stages, i.e., germination/coleoptile elongation (days 1-4) and leaf/root development (days 5-7) in WT rice. However, this process was delayed in cipk15, with only coleoptile elongation being observed up to day 7 (FIG. 2a). We observed that αAmy3 mRNAs increased drastically at day 1 of germination in WT and then declined to basal levels at day 3, and that αAmy8 mRNAs increased and were maintained at high levels up to day 7 in WT embryos/shoots (FIG. 2b). Moreover, in WT, the mRNA levels of CIPK15, ACS1 and ACO5 in embryos/leaves increased from days 1 to 3 and then declined to basal levels at day 5, and they were elevated in roots from day 5 up to day 7. In contrast, in cipk15, increases in mRNA levels for all those genes in both coleoptiles and roots were delayed and maintained at much lower levels up to day 7.

αAmy3 is a highly sensitive gene to sugar starvation and hypoxia^{2,7}, and ACS is generally considered as responsible for the rate-limiting step in ethylene biosynthesis³⁸. Therefore, we have used αAmy3 and ACS1 promoters as markers of CIPK15-activated sugar starvation and ethylene-responsive pathways, respectively. Here, we co-transfected rice suspension cells with Ubi:CIPK15 (effector) and ACS1:LUC or αAmy3:LUC (reporters) and incubated them with or without glucose under aerobic or hypoxic conditions. We found that CIPK15-activated αAmy3 and ACS1 promoter activities were lowest in the presence of both sugar and O₂, increased under either sugar starvation or hypoxia, and were highest under a combination of both sugar starvation and hypoxic conditions Thus, CIPK15 is induced by sugar starvation and hypoxia in a developmentally-regulated biphasic pattern that sequential activates genes necessary for starch-to-sugar conversion and low-level ethylene production during germination/coleoptile development, as well as for high-level ethylene production during leaf and root development under hypoxia (FIG. 2c).

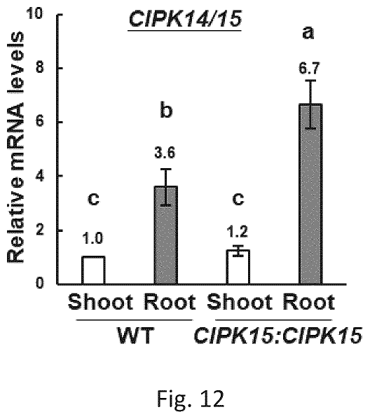

We observed that grain yields of cipk15 mutant plants grown in a rice paddy with regular irrigation were significantly lower than WT (FIG. 2d). We used the CIPK15 native promoter to control the expression of CIPK15 (CIPK15: CIPK15) and found that plants grew normally under aerobic and hypoxic conditions, possibly due to significantly higher and normal CIPK15 expression in roots and shoots, respectively (FIG. 12). We also noted that grain yields of CIPK15-overexpressing lines (carrying CIPK15: CIPK15) grown in the rice paddy increased by 26 to 46% compared to WT (FIG. 2d).

2.4 HXK 5/6 and VDAC1 Interact with CIPK15 and Suppress its Activity on Mitochondria

To identify proteins that interact with CIPK15 and regulate its activity, we extracted total proteins from roots of β-estradiol-treated XVE: CIPK15-dHA transgenic plants grown under aerobic or hypoxic conditions and subjected them to co-immunoprecipitation (co-IP) and proteomics analyses. CIPK15-interacting proteins with top Mascot scores >200 are collected. Most of these proteins were pulled down by CIPK15 under both aerobic and hypoxic conditions, except for HXK5 and HXK6 that were pulled down by CIPK15 only under the aerobic condition.

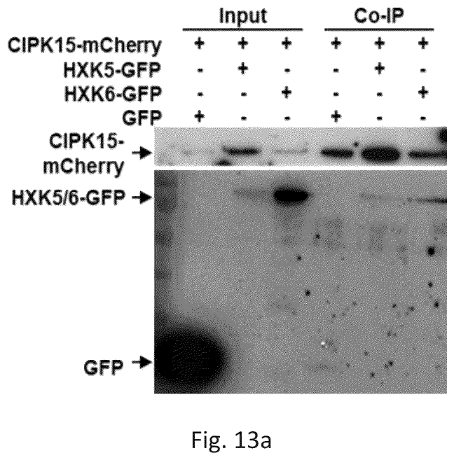

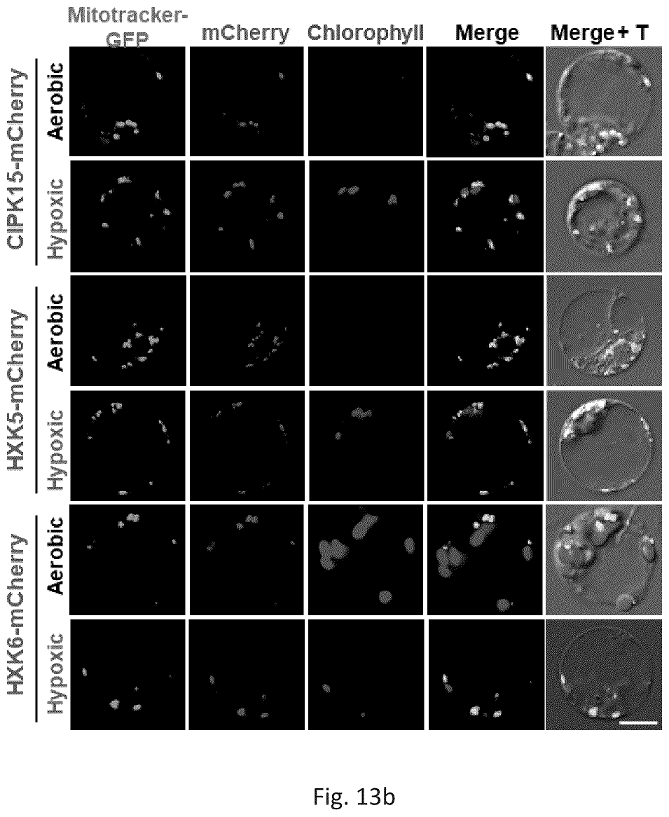

To explore these latter interactions in vivo using co-IP assays, we co-transfected rice protoplasts with Ubi:CIPK15-mCherry and Ubi:HXK5-GFP, Ubi:HXK6-GFP or Ubi:GFP (negative control) constructs. Then, total proteins were extracted before isolating the CIPK15-mCherry protein complex. Our anti-RFP antibodies detected CIPK15-mCherry proteins in all eluents of CIPK15-mCherry co-IP fractions, but anti-GFP antibodies detected HXK5-GFP and HXK6-GFP present in the CIPK15-mCherry complex (FIG. 13a).

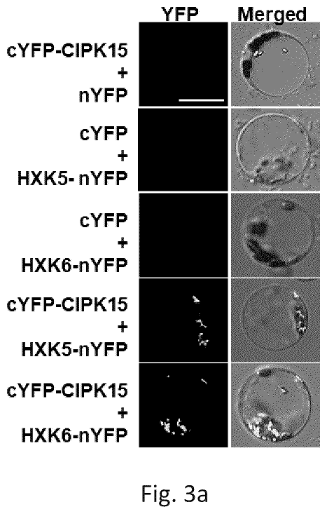

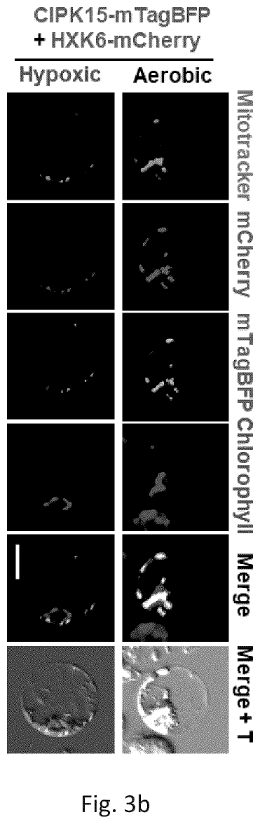

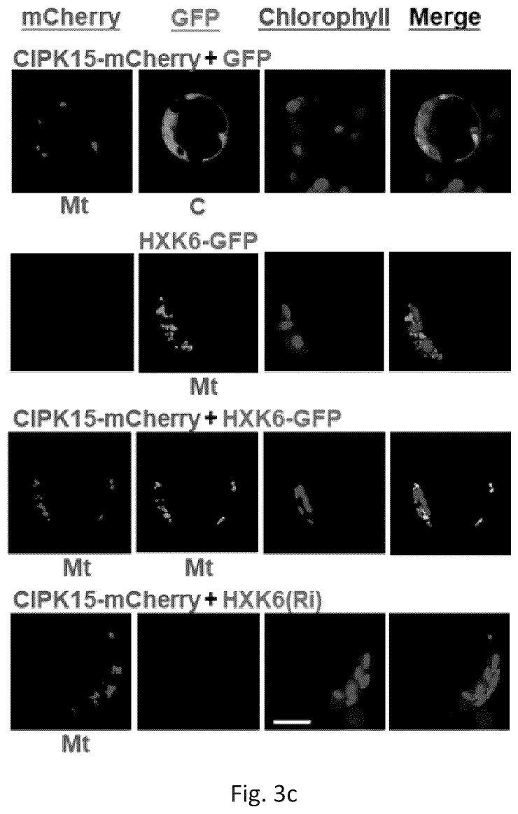

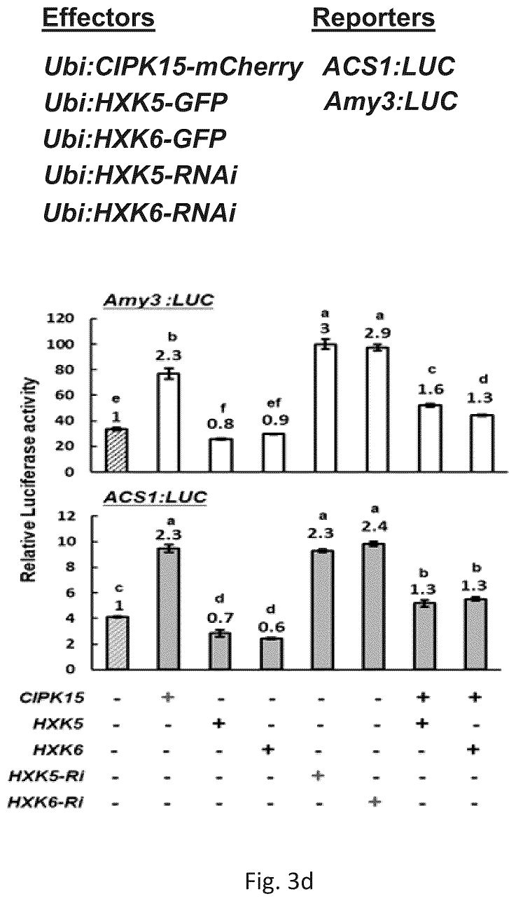

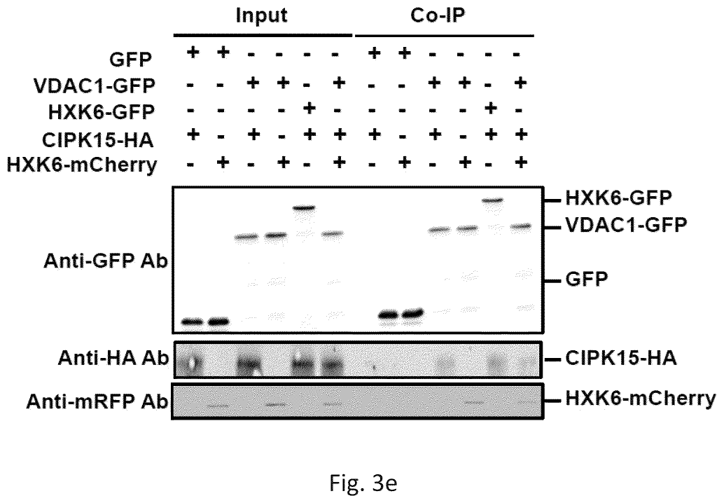

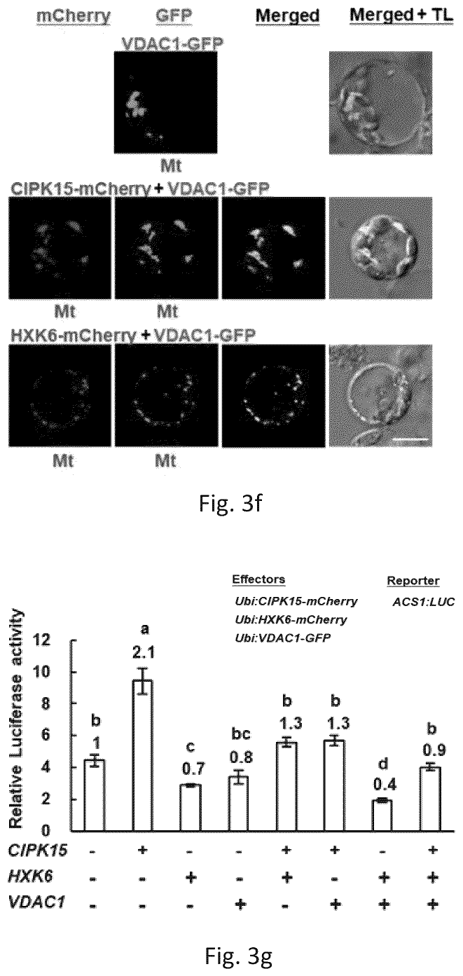

We also performed a bimolecular florescence complementation (BiFC) assay by co-transfecting rice protoplasts with CaMV35S:cYFP-CIPK15 and CaMV35S:HXK5/6-nYFP, which revealed that CIPK15 interacts with HXK5 and HXK6 at mitochondria (FIG. 3a). HXK5/6 are known to associate with mitochondria in rice³⁹. In our transfected rice protoplasts, we observed that HXK5/6-mCherry and CIPK15-mCherry all co-localized with the mitochondrial marker MitoTracker under both aerobic and hypoxic conditions (FIG. 13b). Accordingly, we co-expressed CIPK15-mTagBFP and HXK6-mCherry, or CIPK15-mCherry and HXK6-GFP, and observed that CIPK15 and HXK6 indeed co-localized at mitochondria in rice protoplasts (FIG. 3b, FIG. 3c). CIPK15-mCherry signal was still apparent at mitochondria even when it was co-expressed with HXK6(Ri) (FIG. 3c), indicating that CIPK15 alone is localized on mitochondria.

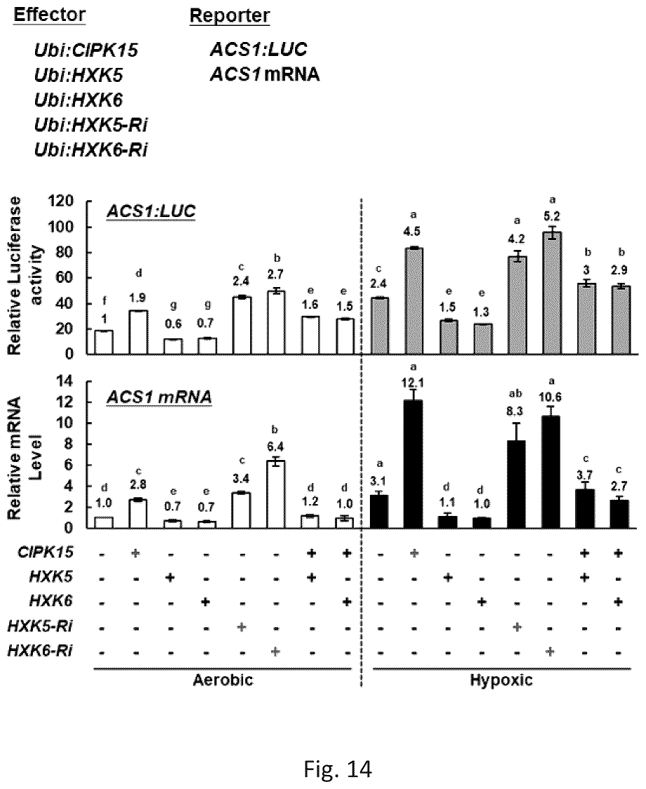

In rice protoplast transient expression assays, the αAmy3 and ACS1 promoters were significantly induced upon overexpression of CIPK15, HXK5-Ri and HXK6-Ri, but they were suppressed by overexpression of HXK5 or HXK6 (FIG. 3d). We obtained similar results for assays carried out under aerobic and hypoxic conditions, but the fold-induction by CIPK15 and HXK5/6-Ri was greater and the degree of CIPK15 suppression by HXK5/6 was alleviated under hypoxia (FIG. 14). These results indicate that HXK5 and HXK6 are both negative regulators of CIPK15.

We also examined other proteins and found that only VDAC1 was predicted to be associated with mitochondria. In a co-IP assay of rice protoplasts co-transfected with Ubi:VDAC1-GFP, Ubi:CIPK15-HA and Ubi:HXK6-GFP or Ubi:HXK6:mCherry, it was revealed that VDAC1 interacted with both HXK6 and CIPK15 (FIG. 3e). We also observed that either CIPK15-mCherry or HXK6-mCherry was co-localized with VDAC1-GFP on the mitochondria of rice protoplasts (FIG. 3f), and the ACS1 promoter was also suppressed by overexpression of VDAC1 (FIG. 3g), supporting that VDAC1-HXK6-CIPK15 formed a complex capable of suppressing CIPK15 activity.

### 2.5 Binding of Glucose to HXK6 is Necessary to Suppress CIPK15 Activity and Inhibit Aerenchyma and Root Development

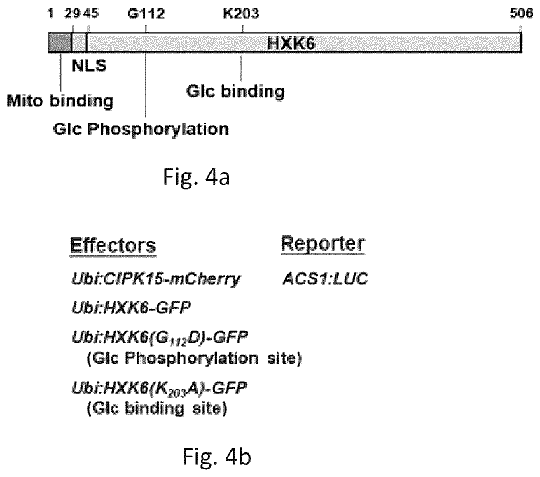

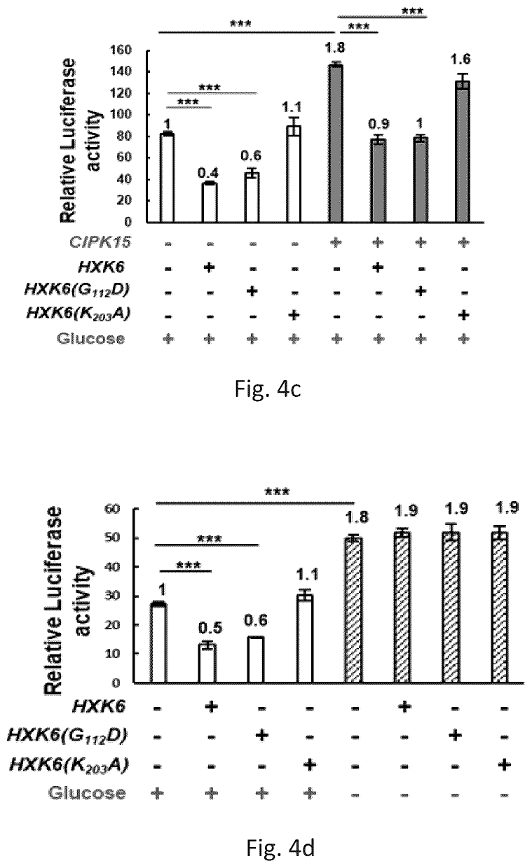

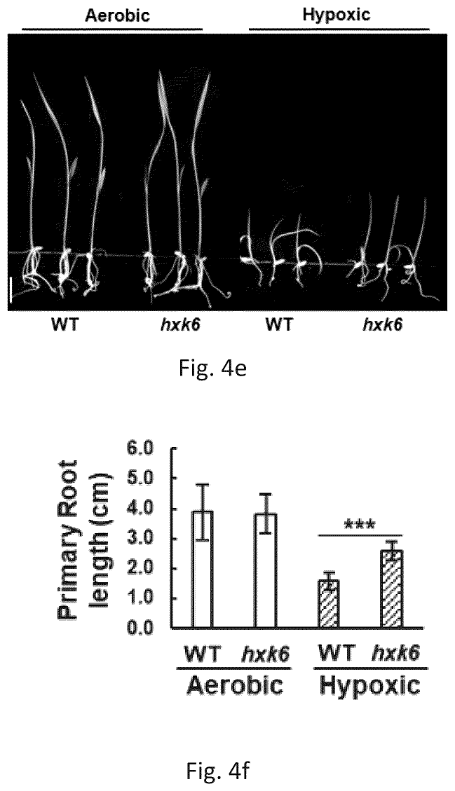

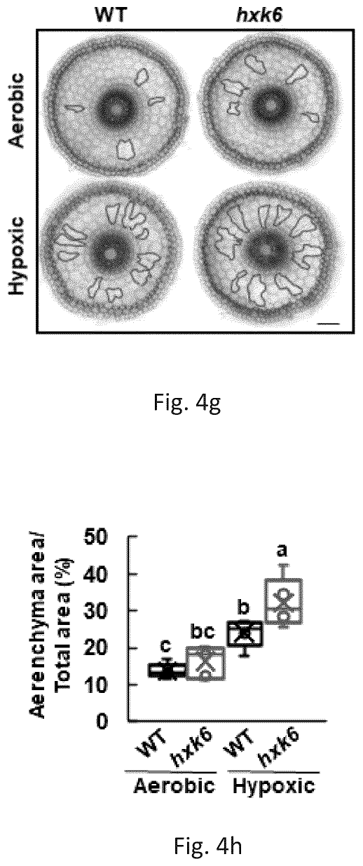

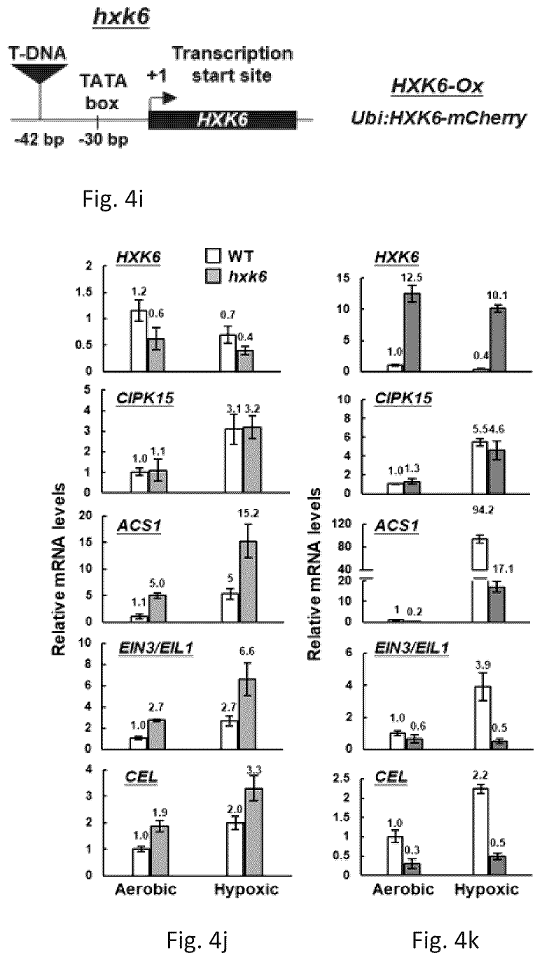

In Arabidopsis and rice, the sugar catabolism and sensing activities of HXKs can be uncoupled^{23,39}. Amino acid residues Gly112 (G112) and Lys203 (K203) in rice HXK6 are essential for glucose phosphorylation and glucose binding, respectively⁴⁰ (FIG. 4a). We generated HXK6 mutant lines in which we changed Lys203 to Ala203 (K₂₀₃A) and Gly112 to Asp112 (G₁₁₂D) (FIG. 4b). We found that the ACS1 promoter was suppressed upon overexpression of HXK6 and HXK6(G₁₁₂D) in rice protoplasts but not of HXK6(K₂₀₃A) (FIG. 4b, FIG. 4c), indicating that HXK6's glucose binding but not glucose phosphorylation is required to suppress the ACS1 promoter. Moreover, neither HXK6 nor HXK6(G₁₁₂D) suppressed the ACS1 promoter in the absence of glucose (FIG. 4b, FIG. 4d), confirming that HXK6's inhibition of the CIPK15-regulated promoter is glucose-dependent.

We obtained a T-DNA-inserted hxk6 mutant and found that the hxk6 homozygous variant was lethal, with only the heterozygous mutant surviving, indicating that HXK6 is indispensable for plant growth. No difference in root growth between the heterozygous mutant and WT was detected under aerobiosis, but root growth was faster for the mutant than for WT under hypoxia (FIG. 4e, FIG. 4f). Moreover, aerenchyma area was larger for the hxk6 mutant relative to WT under hypoxia (FIG. 4g, FIG. 4h). In the hxk6 mutant, the T-DNA is inserted at a position 12 base-pairs (bp) upstream of the TATA box (FIG. 4i). Notably, HXK6 mRNA levels were reduced by only 50% in roots of the hxk6 heterozygous mutant under both aerobic and hypoxic conditions, but ACS1, EIN3/EL1 and CEL mRNAs were all significantly enhanced (FIG. 4j). We also found that no transgenic plant overexpressing HXK6 (HXK6-Ox) could regenerate from transgenic calli, so we used the transgenic calli for further study. In those HXK6-Ox cells, the accumulations of ACS1, EIN3/EL1 and CEL mRNAs induced by hypoxia were significantly reduced relative to WT (FIG. 4k). Furthermore, CIPK15 mRNA levels were equivalent to WT in both hxk6 root and HXK6-Ox cells (FIG. 4j, FIG. 4k). These findings confirm that HXK6 suppresses CIPK15-regulated ethylene signaling and root and aerenchyma development.

2.6 the NAF Domain is Essential for CIPK15 Activity, Mitochondrial Localization and Interaction with HXK6.

The CIPK family is typically composed of an N-terminal catalytic/kinase domain containing an activation loop, and a C-terminal regulatory domain containing the autoinhibitory Asn-Ala-Phe (NAF) domain, with the NAF domain directly interacting with the catalytic domain to inhibit kinase activity⁴¹. To define the functional domains in CIPK15 (FIG. 5a), we introduced mutations in CIPK15 at predicted functional regions, generating NAF-domain-deleted (CIPK15ΔNAF) variant, as well as constitutively phosphorylated and dephosphorylated Thr in the activation loop by replacing it with Asp (CIPK15T₁₇₁D) and Ala (CIPK15T₁₇₁A), respectively. These CIPK15 mutants were fused individually to the mCherry reporter (FIG. 5b). Then, we determined ACS1 mRNA levels in rice protoplasts after transfecting CIPK15 mutants with or without HXK6. We found that in the absence of HXK6, CIPK15ΔNAF and CIPK15(T₁₇₁A) did not enhance ACS1 mRNA levels, whereas they were 2.3- and 3.9-fold higher for WT CIPK15 and CIPK15(T₁₇₁D), respectively (FIG. 5b). In the presence of HXK6, ACS1 mRNA levels were reduced in cells transfected with WT and all CIPK15 mutants, indicating endogenous CIPK15 was also likely suppressed. These experiments indicate that the NAF domain is required for full CIPK15 functionality, that phosphorylation at the CIPK15 kinase active site promotes CIPK15 activity. We performed co-IP coupled with liquid chromatography-tandem mass spectrometry (LC MS/MS) and observed that CIPK15(T₁₇₁) was phosphorylated in vivo only under hypoxia (FIG. 5c).

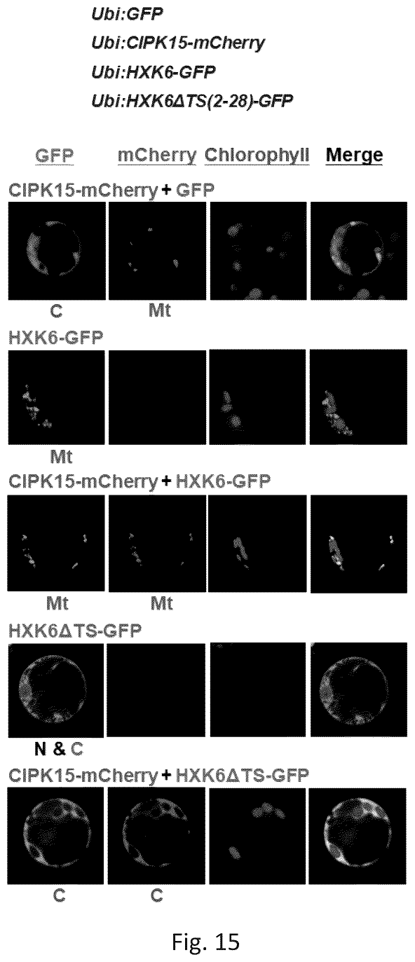

The NAF domain of the CIPK protein family comprises 23-24 amino acids in Arabidopsis and rice, and it contains several highly conserved hydrophobic amino acids (FIG. 15). To identify amino acid residues in the NAF(₂₉₈₋₃₃₃) domain that are essential for CIPK15 function, we substituted the three core amino acids Asn-Ala-Phe (NAF₃₁₅₋₃₁₇) with Pro to generate CIPK15(NAF₃₁₅₋₃₁₇PPP), and another two highly conserved amino acids Leu₃₂₈ and Phe₃₃₂ with Pro to generate CIPK15(L₃₂₈P/F₃₃₂P). By means of transient expression assay, we found that neither of these CIPK15 NAF mutants nor the NAF-deleted variant (CIPK15ΔNAF) could activate the ACS1 promoter (FIG. 5d). Moreover, the cellular localization of all of these CIPK15 NAF mutants fused to mCherry shifted from mitochondria to the cytoplasm and nucleus in the presence of HXK6 (FIG. 5e).

HXK6 (K₂₀₃A) that lacks glucose-binding activity cannot suppress CIPK15 activity (FIG. 4c, FIG. 4d). We observed that HXK6 (K₂₀₃A) localized on mitochondria and that CIPK15-mCherry co-expressed with HXK6 (K₂₀₃A) also localized there (FIG. 5e) and remained active (FIG. 5f). However, CIPK15ΔNAF-mCherry co-expressed with HXK6 (K₂₀₃A)-GFP switched localization to the cytoplasm and nucleus (FIG. 5e) and became inactive (FIG. 5f). Thus, the NAF domain appears to be necessary for CIPK15 activity, mitochondrial localization, and physical interaction with HXK6.

2.7 OsCIPK15:CIPK15 Improves Flooding Tolerance in S. viridis

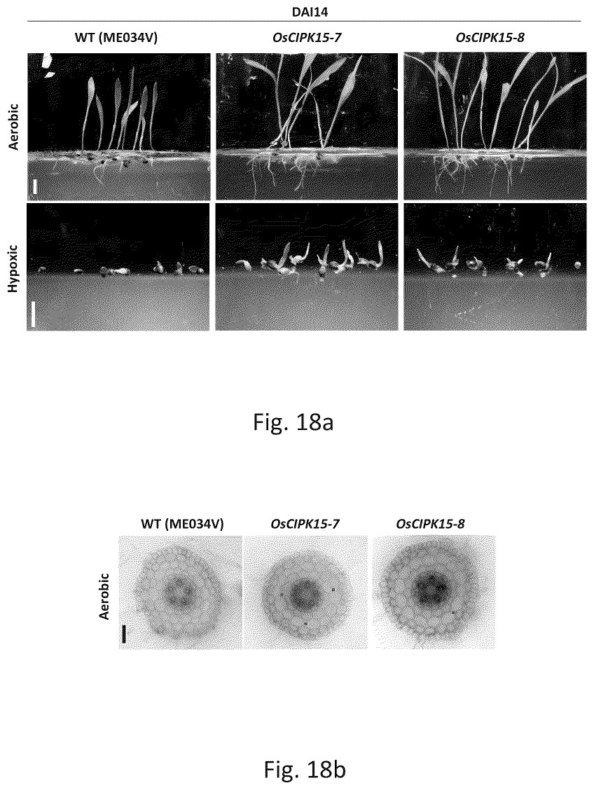

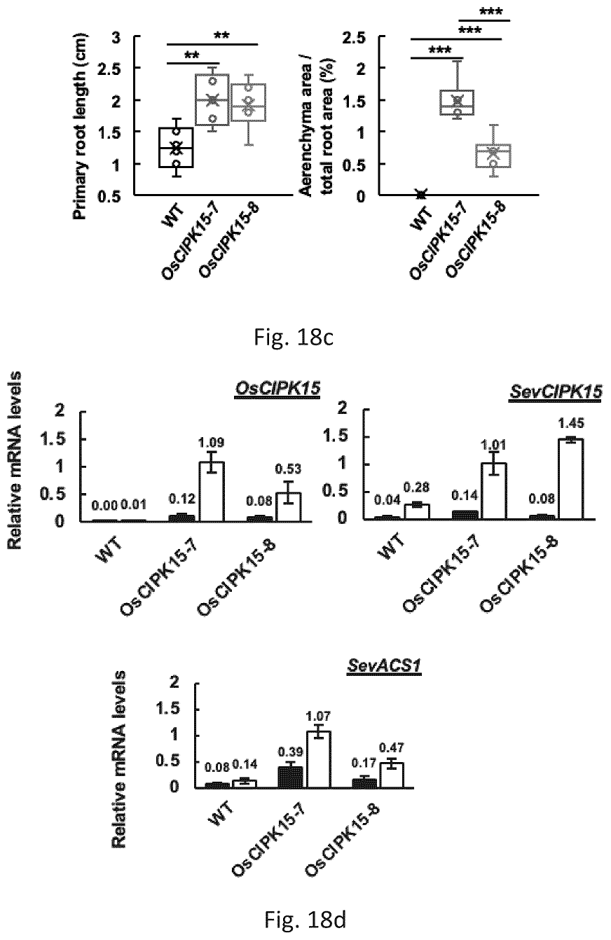

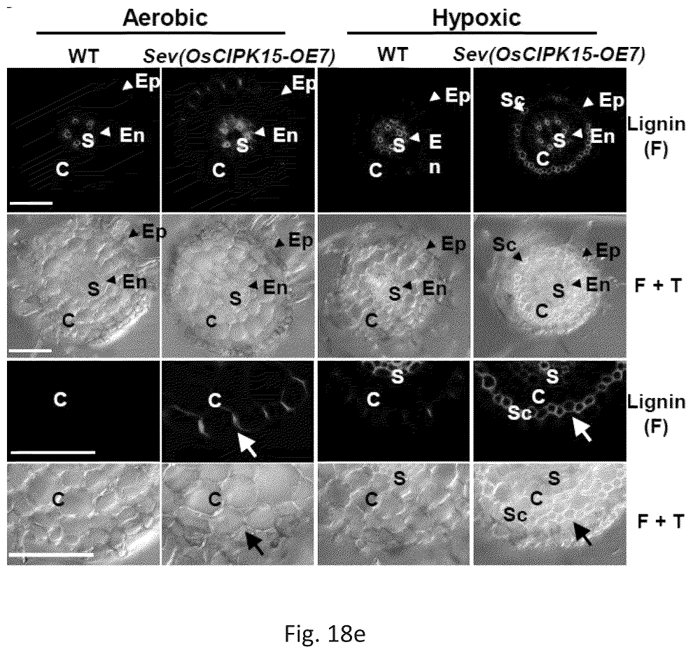

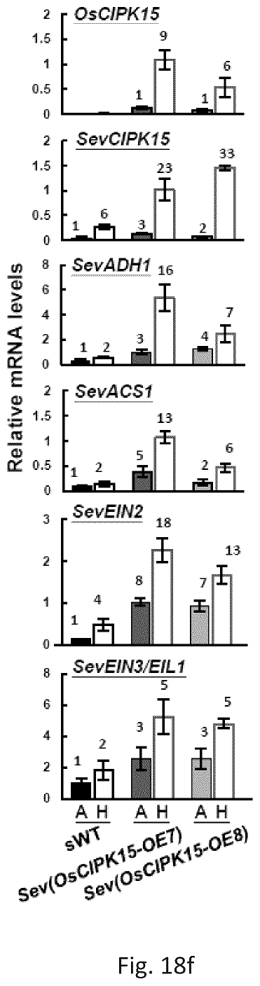

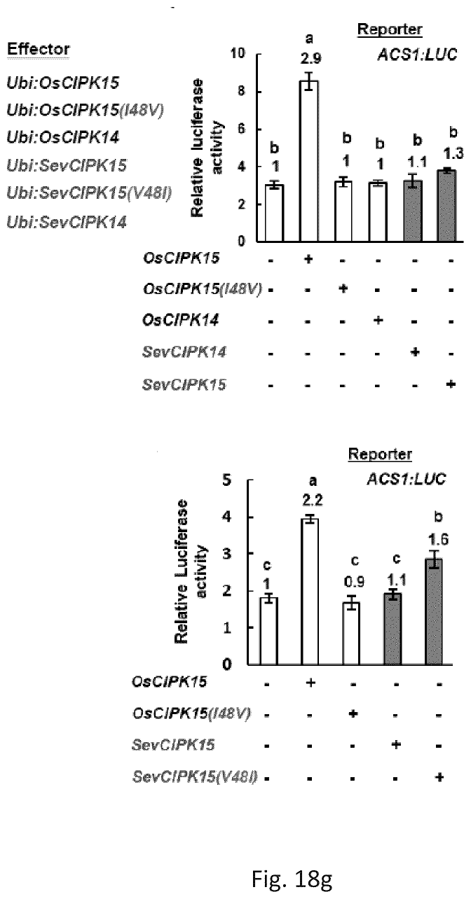

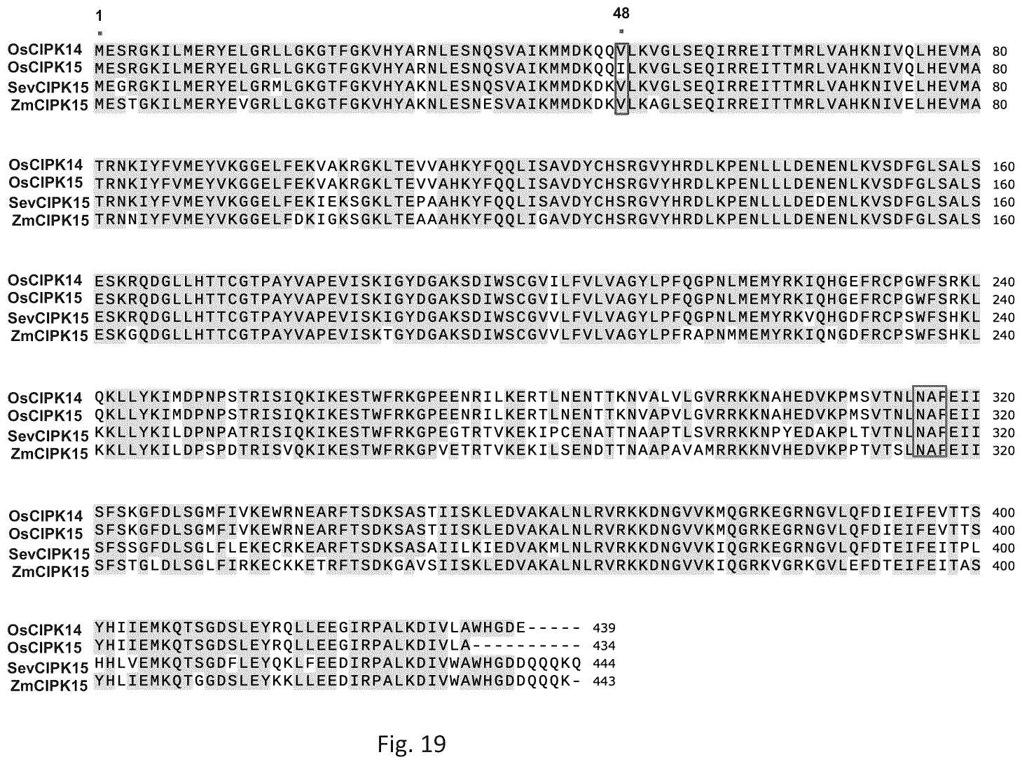

Above findings demonstrated that OsCIPK15 is essential for the germination and root development under hypoxia in rice. To test whether OsCIPK15 is also able to confer flooding tolerance to other cereals, we transformed the plasmid OsCIPK15:OsCIPK15 into a flooding-intolerance species: S. viridis. Our results showed that, under aerobic conditions, the WT (cv. ME034V) exhibited shorter primary roots, and almost no aerenchyma development, whereas transgenic plants had longer roots and more aerenchyma along its primary roots. (FIGS. 18a to 18c). Under hypoxia, the WT showed a sensitive phenotype to low O2, with no elongated coleoptile rate at DAI14 under hypoxia. In contrast, the transgenic plants had elongated coleoptiles and shoot/root development at 14 DAI under hypoxia (FIG. 18a). We also found that lignin deposition was enhanced in the wall of root cortex cells near epidermis compared to WT under aerobiosis, and significantly enhanced in newly developed sclerenchyma in the nodal root of the Sev(OsCIPK15-OE) line, which was absent in WT under hypoxia (FIG. 18e). The results indicate that OsCIPK15 is able to improve flooding tolerance in other cereals.

qRT-PCR analysis showed that OsCIPK15 was significantly induced under hypoxia in two transgenic plants, but almost undetectable in the WT. SevCIPK15 expressions were also induced by hypoxia, and could be further induced in the OsCIPK15 transgenic plants, suggesting a possible positive feedback regulation by OsCIPK15. The expression of SevACS1 was similar to that of OsCIPK15, which was induced by hypoxia and showed higher expression in the transgenic plants (FIG. 18d), indicating that OsCIPK15 could regulate similar pathways in S. viridis and rice. Furthermore, levels of endogenous Setaria ADH1, ACS1, EIN2 and EIN3/EIL1 mRNAs, reflecting the fermentation and ethylene biosynthesis/signaling pathways, in Sev(OsCIPK15-OE) lines were also increased under aerobiosis, and even more significantly increased under hypoxia (FIG. 18f). This outcome indicates that SevCIPK15 may have a reduced capacity to activate downstream genes essential for germination and root growth under hypoxia.

In addition, to uncover the unique features of OsCIPK15, we compared the protein sequences from OsCIPK15 with those of its orthologues from other Poaceae cereals. Alignment results showed that Ile-48 from OsCIPK15 is uniquely present in the Oryza genus, the other ortholog proteins containing Val-48 (FIG. 19) or Ala-48 (e.g. in Leerisa perrieri, LpCIPK15, SEQ ID NO: 15). The Thr-171 phosphorylation site, Pro-292, and the conserved residues in the NAF domains (NAF315-317, L328, F332) of OsCIPK15 were shared the same amino acids among all evaluated species (FIG. 19). Consequently, we substituted Ile 48 with Val (148V) in rice CIPK15, and a subsequent rice protoplast transient expression assay revealed that, unlike for rice WT CIPK15, CIPK15(I48V) and CIPK14 in rice and both CIPK14 and CIPK15 in Setaria could not activate the rice ACS1 promoter (FIG. 18g). We also substituted Val 48 with Ile (V48I) in Setaria CIPK15, and the resulting Setaria CIPK15(V48I) variant could activate the rice ACS1 and αAmy3 promoters (FIG. 18g), although only to a level 70% that elicited by rice WT CIPK15. These results indicate that the Ile48 residue is essential for rice CIPK15 to activate ethylene biosynthesis.

### 3.Discussion

Plant root architecture and function are essential aspects of water and nutrient uptake, anchorage, and interactions with microbes in soil, all of which are related to processes that impact growth rate, abiotic stress tolerance and productivity. Underwater root growth is critical to plants surviving flooding conditions. Here, we have deciphered a unique mechanism employed by rice for hypoxia tolerance. We show that the mitochondria-associated CIPK15-HXK5/6-VDAC1 complex regulates carbohydrate metabolism, ethylene and auxin biosynthesis/transport, root anatomy, and root development for flooding adaptation in rice, ultimately improving grain yield in a rice paddy.

### 3.1 Multifaceted Roles of CIPK15 in Regulating the Hypoxia Tolerance of Rice

Two unique features enable CIPK15 to play a key role in flooding tolerance in rice. First, CIPK15 regulates ethylene biosynthesis in roots under hypoxia. Ethylene is produced by all plant cells and, as a gas, it quickly diffuses and is sensed at production sites. Due to restricted gas diffusion and O₂ exchange under water, ethylene concentrations increase rapidly in submerged plant tissues, initiating ethylene signaling to regulate genes responsive to flooding⁴². The mechanisms by which ethylene biosynthesis is regulated under hypoxia had previously been unclear. In rice, increased expression of CUT1-LIKE (CUT1L, a homolog of CUT1 required for cuticular wax production in Arabidopsis) induces accumulations of very-long-chain fatty acids, and REDUCED CULM NUMBER (RCN1, an ATP-binding cassette transporter) enhances ACS1 and ACO5 expression, both of which serve to promote ethylene accumulation and aerenchyma formation in rice roots under hypoxia⁴³. We found that CIPK15 upregulates ACS, ACO, CUT1L and RCN1 expression in rice seedlings under hypoxia (FIG. 1i), suggesting that CIPK15 is an upstream regulator of ethylene biosynthesis. Rice displays greater ACS and ACO expression, produces more ethylene, and develops more aerenchyma in roots than maize when both are grown in stagnant water¹⁹, indicating that the CIPK15-upregulated ethylene biosynthesis pathway constitutes a unique mechanism for supporting rice root growth under hypoxia.

Second, CIPK15 senses sugar and O₂ deficiency and reprograms plant metabolism and development to adapt to flooding. As seedlings develop, the expression and function of CIPK15 itself is regulated by sugar starvation and hypoxia in a developmentally-regulated biphasic pattern (FIG. 2a, FIG. 2b). Under submergence conditions, CIPK15 regulates the expression of a cohort of genes essential for sugar production, ethylene and auxin biosynthesis, transport and signaling, and aerenchyma and root development in developing rice seedlings. In early developmental phases, starch hydrolysis by □-amylases is crucial for providing the sugars and energy necessary for germination and shoot production and, in later phases, ethylene-promoted aerenchyma formation is essential for root development. Aerenchyma development couples vertical root elongation and radial expansion (FIGS. 1c to 1h and FIG. 9). Replacement of living cortical cells with aerenchyma that more readily transmit air may satisfy the respiratory and nutrient requirements of root tissues, enabling metabolically-efficient root growth⁴⁴. Increased root diameter has also been linked to greater root bending stiffness and root penetration of strong physical soil barriers⁴⁵. Rice is cultivated in stagnant water primarily to stave off pests and limit weed growth in the rice paddy. CIPK15-regulated aerenchyma development in rice ensures vertical gas exchange in roots to facilitate root growth in the hypoxic water-logged soil. The cipk15 mutant grew poorly in waterlogged soil but normally in drained soil², indicating that CIPK15 is indispensable for root growth under hypoxia. In contrast, we observed vigorous root growth of transgenic rice overexpressing CIPK15, presumably caused by better aeration due to aerenchyma formation, leading to significantly increased grain yield relative to WT (by 26-54%) in the water-logged soil of a rice paddy (FIG. 2d).

### 3.2 CIPK15-HXK5/6-VDAC1 Interaction Regulates Root Development Under Aerobic and Hypoxic Environments

A previous phylogenetic analysis revealed that CIPK14/15 in rice formed a clade distinct from other CIPKs in rice and Arabidopsis⁴¹, implying that rice CIPK15 may exert unique functions different from those of other CIPKs. Our study demonstrates that the core Asn-Ala-Phe amino acids in the NAF domain are necessary for CIPK15 activity and association with mitochondria and HXK6. In Arabidopsis, CBL-CIPK interaction controls transport of various ions across the plasma and vacuolar membranes⁴⁶, and the NAF domain of CIPK is responsible for interacting with CBL and also serves as a kinase autoinhibitory region 47.

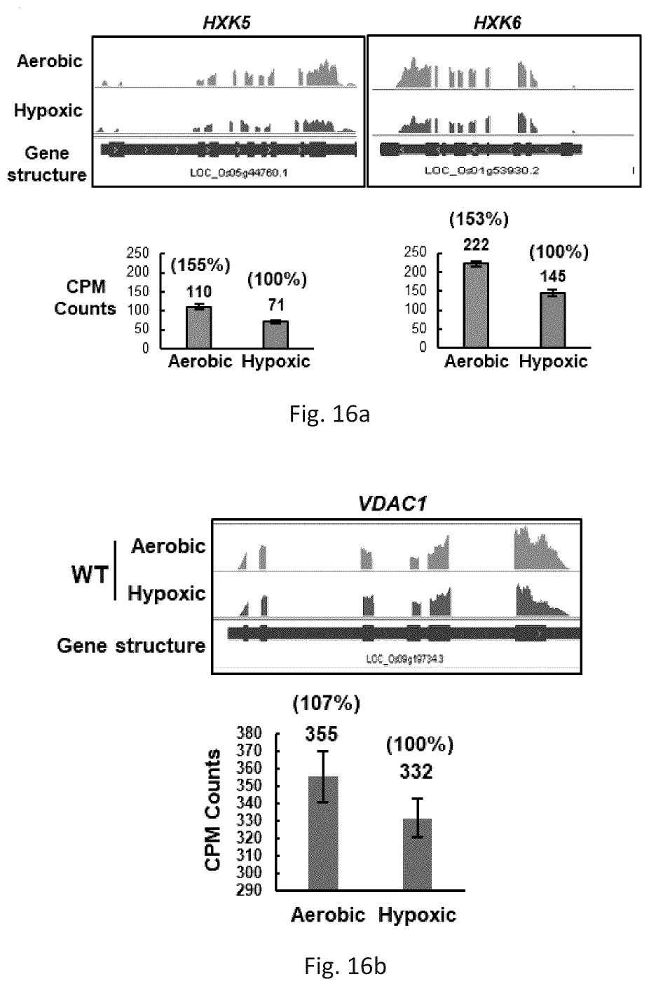

VDAC, a mitochondrial porin, is the most abundant protein of the mitochondrial outer membrane and is the major channel for the exchange of metabolites and ions between mitochondria and other cellular compartments²⁹. In animal cells, VDAC1 interacts with partner proteins to regulate mitochondrial functions. Binding of VDAC1 to HXK provides a metabolic benefit and apoptosis-suppressive capacity to many cancer cells that endows them with a proliferative advantage and increased resistance to chemotherapy 28. The N-terminal region (residues 1-15) of animal HXKII is required for its mitochondrial localization and interaction with VDAC1²⁹. We demonstrate herein that rice VDAC1 interacts and cooperates with HXK6 to suppress CIPK15 activity (FIG. 3f, FIG. 3g). The N-terminal region (residues 1-28) of HXK5/6 also contains a mitochondrial targeting signal (TS)³⁹. We deleted the TS of rice HXK6 and fused it to GFP (HXK6ΔTS-GFP) and observed its localization in cytoplasm and, furthermore, that co-expressed CIPK15-mCherry and HXK6ΔTS-GFP also co-localized in cytoplasm (FIG. 16). Thus, a region other than the TS of HXK6 interacts with the CIPK15 NAF domain in the cytoplasm to prevent CIPK15 from being associated with mitochondria.

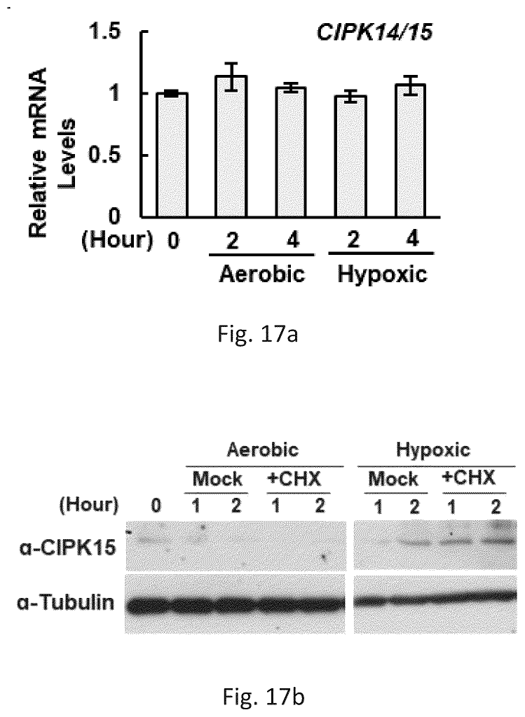

In WT rice, transcript levels of CIPK15 increased by ˜24% under hypoxia compared to aerobic conditions (FIG. 11a). In contrast, the transcript levels of HXK5 and HXK6 were reduced by 53-55%, and of VDAC1 slightly reduced by 7%, under hypoxia compared to aerobic conditions (FIG. 17a, FIG. 17b). Additionally, CIPK15 protein proved more stable under hypoxia. These results indicate that more CIPK15 but less HXK5/6 is present on mitochondria under hypoxia. In contrast, less CIPK15 but more HXK5/6 is present on mitochondria under aerobic conditions. It may explain why HXK5/6 was pulled down by CIPK15 only under aerobiosis under our experimental conditions. Furthermore, the absolute CIPK15 mRNA level is higher than HXK6 mRNA under hypoxia (compare FIG. 11a and FIG. 12a), suggesting there could be more active CIPK15, i.e., not being suppressed by interacting with HXK5/6, on mitochondria under hypoxia.

CIPK15 is required for aerenchyma and root development, as revealed by aerenchyma area being significantly reduced (FIG. 1d, FIG. 1e) and root growth being delayed in the cipk15 mutant under aerobic conditions (FIG. 9a). Enhancing whole-plant expression of CIPK15 (10.1-12.5-fold under the control of the strong constitutive Ubi promoter) (FIG. 4k), severely inhibited plant growth, yet 2-fold-increased CIPK15 expression in roots via its native promoter (FIG. 12) enhanced plant growth and grain yield (FIG. 2d). This outcome implies that a moderate increase in CIPK15 expression in roots and limited CIPK15 expression in whole plants can benefit plant growth. Taken together, our findings demonstrate that CIPK15 is important for root growth, but its transcription is downregulated by O₂ and its activity is restricted by HXK5/6 under aerobic conditions, whereas elevated expression of CIPK15 is required for root growth under hypoxia. This scenario also explains why growth of the cipk15 mutant is only slightly affected under aerobic condition but is severely affected under hypoxia. In WT, although the expression of CIPK15 and its downstream genes is upregulated under hypoxia (FIG. 1i), roots still elongate more rapidly under aerobic conditions (FIG. 1c, FIG. 1f), suggesting additional factors also enhance root growth under aerobic conditions.

HXKs catalyze the ATP-dependent phosphorylation of glucose to glucose-6-phosphate. A previous protein structure-based study demonstrated that rice HXK6 adopts an open inactive state in the absence of glucose, but is rearranged into a closed active state upon binding glucose⁴⁰. We show herein that glucose binding, likely a closed yet active state, but not the phosphorylation of glucose by HXK6, suppresses CIPK15 activity. HXK5 and HXK6 share similar features, e.g., localization on mitochondria, interaction with CIPK15, and suppression on CIPK15 activity. HXK5 is a major HXK functioning during pollen germination and tube growth, with HXK5 knockout mutant plant being able to grow yet male sterile⁴⁸. In the present study, the HXK6 homozygous knockout mutant seedling is unable to grow, indicating HXK6 is necessary for hexose metabolism during plant growth.

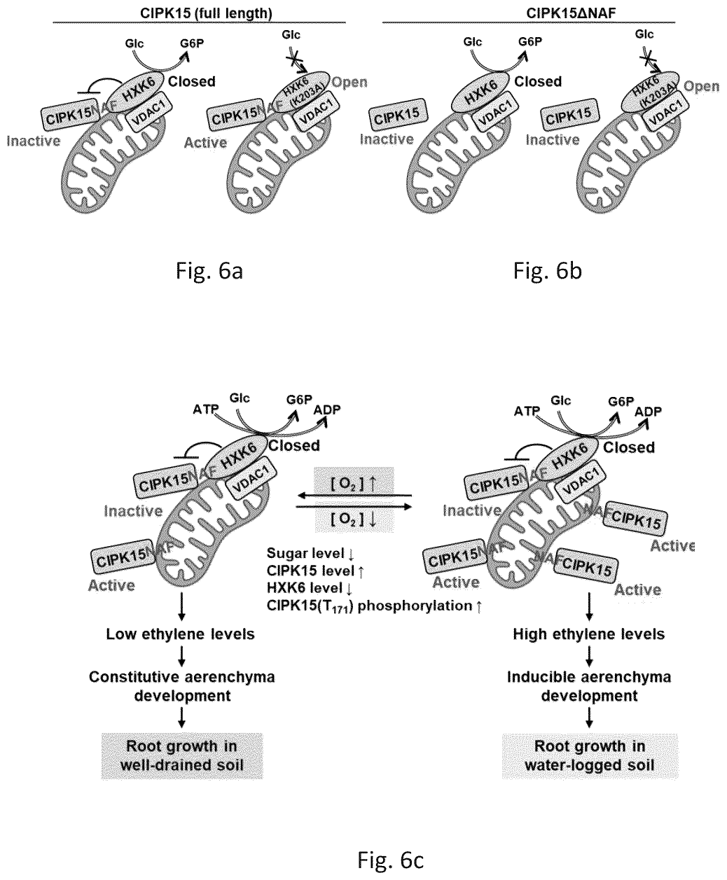

Accordingly, we propose a model to illustrate the function of the HXK6-CIPK15-VDAC1 interaction on mitochondria and how it regulates glucose-dependent rice root development. Glucose-bound HXK6 adopts a closed state that suppresses CIPK15 activity (FIG. 6a). The HXK6(K₂₀₃A) mutant that cannot bind glucose is in an open state that allows CIPK15 to remain active (FIG. 6b). CIPK15 lacking its NAF motif dissociates from mitochondria and is inactive, regardless of whether HXK6 is in the closed or open state on mitochondria (FIG. 6c).

In summary, the elevated O₂ concentrations associated with well-drained soil allow HXK5/6-VDAC1 to accumulate and suppress CIPK15 activity, leading to maintenance of minimal levels of active CIPK15 on mitochondria, low ethylene accumulation, and constitutive aerenchyma development (FIG. 6d). In contrast, as O₂ levels decline in water-logged soil, glycolysis and fermentation reduce sugar reserves, CIPK15 levels increase, and HXK5/6 and VDAC1 levels decline, thereby limiting the CIPK15-HXK5/6-VDAC1 interaction, hence less CIPK15 being inactivated. Together, these factors contribute to greater accumulations of active CIPK15 on mitochondria, enhancing ethylene biosynthesis, which promote root growth under hypoxia (FIG. 6e). These factors in rice render it unique among cereals and other major crops in being capable of tolerating flooding.

### 3.3 OsCIPK15 can Confer Flooding Tolerance in Other Species

Rice bears unique features that have permitted it to adapt to hypoxic environments. OsCIPK15 is the key gene that is essential for anaerobic germination and hypoxic root growth. In this chapter, we report the first successful case of conferring flood tolerance to a flooding-sensitive species into tolerant by transforming rice OsCIPK15 into S. viridis.

The root architecture of S. viridis differs from that of rice, which lacks of sclerenchyma⁴⁹, and carries fewer layers of cortex⁵⁰. To date, no studies have described aerenchyma formation in S. viridis, but studies examining the acclimated species Setaria italica have shown that it exhibits low level of aerenchyma development compared to other waterlogging-tolerant-speciessis^{51,52}. We demonstrated that seminal roots were longer in OsCIPK15 transgenic lines than the seminal roots in the WT (FIG. 18a). Although the percentage of aerenchyma area was also higher in the transgenic plants, this percentage was still much lower than that found in rice under aerobic conditions (FIG. 18b). Since we cannot exclude the possibility that low level of aerenchyma development in S. viridis was due to the plants being young seedlings, an additional assay with mature seedlings is needed.

Underwater seedling development can be classified into coleoptile elongation and shoot/root development stages (FIG. 2a). Though WT seeds of S. viridis were able to germinate under water, they failed to elongate its coleoptile by 14 DAI (FIG. 18a), suggesting that S. viridis is sensitive to low oxygen. In contrast, transgenic plants carrying OsCIPK15 were able to develop true leaves and elongated coleoptiles under hypoxia (FIG. 18a). We also noticed that seedlings and roots of the transgenic plants were unable to further developed after DAI14, indicating that some factors related to underwater development are still limiting in S. viridis.

The importance of ethylene accumulation to underwater seedling development has been previously described⁵³. In previous chapters, we have shown that OsCIPK15 is the key regulator for the ethylene biosynthesis in rice in previous chapters. Here, we further demonstrate that the expression profiles of OsCIPK15 are correlated with the expression of SevACS1 and the phenotypes of S. viridis. OsCIPK15-7 showed the highest expression level of OsCIPK15, and also exhibited the best seedling growth under hypoxia, while OsCIPK15-8 expressed mild levels of OsCIPK15 and SevACS1, remaining at the coleoptile elongation stage at 14 DAI under hypoxia (FIG. 18f). Interestingly, we observed that SevCIPK15 may also be induced by OsCIPK15, in which showed 4- to 5-fold induction in two transgenic lines under hypoxia. Although the expression levels of SevCIPK15 can still be induced by hypoxia, it does not correlate with the expression patterns of OsCIPK15 and SevACS1 (FIG. 18f), indicating that SevCIPK15 may not function in S. viridis.

Our sequence alignment results suggested that CIPK15 is highly conserved across the Poaceae family, showing >80% similarity among the orthologue proteins (FIG. 19). Important amino acid residues identified in the previous chapter (I48, T171, P292, NAF315-317, F328, and L332) were almost identical among the orthologue proteins, with the exception of Ile-48, which only appears in the Oryza genus (FIG. 19). In the previous chapters, we have shown that replacement of Ile-48 with Val-48 in OsCIPK15 [OsCIPK15(148V)] abolished OsACS1 promoter activity under hypoxia (FIG. 11), indicating that CIPK15 orthologs carrying Val-48 may not have the same function as OsCIPK15 under hypoxia.

In summary, our study demonstrated that OsCIPK15 can confer flooding tolerance in other species. We showed that OsCIPK15 carries the amino acid residue Ile-48, which allows it to regulate ethylene-dependent aerenchyma development. To date, no other studies have successfully improved underwater seedling development in a plant species other than rice. Our study offers an important strategy for improvement of flooding tolerance in plants, and also reveals a new insight into how rice possesses superior flooding tolerance under an evolutionary lens.

>OsCIPK15 (Oryza sativa Os12g0113500)
SEQ ID NO: 1
MESRGKILMERYELGRLLGKGTFGKVHYARNLESNQSVAIKMMDKQQILKVGLSEQIRREIT
TMRLVAHKNIVQLHEVMATRNKIYFVMEYVKGGELFEKVAKRGKLTEVVAHKYFQQLISAVD
YCHSRGVYHRDLKPENLLLDENENLKVSDFGLSALSESKRQDGLLHTTCGTPAYVAPEVISK
IGYDGAKSDIWSCGVILFVLVAGYLPFQGPNLMEMYRKIQHGEFRCPGWFSRKLQKLLYKIM
DPNPSTRISIQKIKESTWERKGPEENRILKERTLNENTTKNVAPVLGVRRKKNAHEDVKPMS
VTNLNAFEIISFSKGFDLSGMFIVKEWRNEARFTSDKSASTIISKLEDVAKALNLRVRKKDN
GVVKMQGRKEGRNGVLQFDIEIFEVTTSYHIIEMKQTSGDSLEYRQLLEEGIRPALKDIVLA
>ObCIPK15 (Oryza barthii OBART11G00920)
SEQ ID NO: 2
MESRGKILMERYELGRLLGKGTFGKVHYARNLESNQSVAIKMMDKQQILKVGLSEQIRREIT
TMRLVAHKNIVQLHEVMATRNKIYFVMEYVKGGELFEKVAKRGKLTEVVAHKYFQQLISAVD
YCHSRGVYHRDLKPENLLLDENENLKVSDFGLSALSESKRQDGLLHTTCGTPAYVAPEVISK
IGYDGAKSDIWSCGVILFVLVAGYLPFQGPNLMEMYRKIQHGEFRCPGWFSRKLQKLLYKIM
DPNPSTRISIQKIKESTWERKDPEENRILKERTLNENTTKNVAPVLGVRRKKNAHEDVKPMS
VINLNAFEIISFSKGFDLSGMFIVKEWRNEARFTSDKSASTIISKLEDVAKALNLRVRKKDN
GVVKMQGRKEGRNGVLQFDIEIFEVTTSYHIIEMKQTSGDSLEYRQLLEEGIRPALKDTVLA
>OgCIPK15 (Oryza glaberrima ORGLA11G0011100)
SEQ ID NO: 3
MESRGKILMERYELGRLLGKGTFGKVHYARNLESNQSVAIKMMDKQQILKVGLSEQIRREIT
TMRLVAHKNIVQLHEVMATRNKIYFVMEYVKGGELFEKVAKRGKLTEVVAHKYFQQLISAVD
YCHSRGVYHRDLKPENLLLDENENLKVSDFGLSALSESKRQDGLLHTTCGTPAYVAPEVISK
IGYDGAKSDIWSCGVILFVLVAGYLPFQGPNLMEMYRKIQHGEFRCPGWFSRKLQKLLYKIM
DPNPSTRISIQKIKESTWERKGPEENRILKERTLNENTTKNVAPVLGVRRKKNAHEDVKPMS
VTNLNAFEIISFSKGFDLSGMFIVKEWRNEARFTSDKSASTIISKLEDVAKALNLRVRKKDN
GVVKMQGRKEGRNGVLQFDIEIFEVTTSYHIIEMKQTSGDSLEYRQLLEEGIRPALKDIVLA
>OrCIPK15 (Oryza rufipogon ORUFI11G00820)
SEQ ID NO: 4
MESRGKILMERYELGRLLGKGTFGKVHYARNLESNQSVAIKMMDKQQILKVGLSEQIRREIT
TMRLVAHKNIVQLHEVMATRNKIYFVMEYVKGGELFEKVAKRGKLTEVVAHKYFQQLISAVD
YCHSRGVYHRDLKPENLLLDENENLKVSDFGLSALSESKRQDGLLHTTCGTPAYVAPEVISK
IGYDGAKSDIWSCGVILFVLVAGYLPFQGPNLMEMYRKIQHGEFRCPGWFSRKLQKLLYKIM
DPNPSTRISIQKIKESTWERKGPEENRILKERTLNENTTKNVAPVLGVRRKKNAHEDVKPMS
VINLNAFEIISFSKGFDLSGMFIVKEWRNEARFTSDKSASTIXILMSQAFSFRSSNSLGFSS
NEFICLACVLFSIYVFVSGEFMLRQCMQLYLES
>BdCIPK15 (Brachypodium distachyon BRADI_4g25880v3)
SEQ ID NO: 5
MDSSGKVVLGRYEVGRLLGKGAFGKVHYAKNLKSNRSVAIKMLDKEKVLKVGLSEQVRREIT
TMRLVAHKNIVKLHEVMATRSKIYFVMEYVKGGQLEDKISNGGKLTESAAHKYFQQLISAVD
YCHSRGVYHRDLKPENLLLDEHENLKVSDFGLSALSESKRQDGLLHTICGSPAYVAPEVISK
GGYDGAKSDIWSCGVVLFVLVAGYLPFQGQNLMEMYMKIEQGDFKCPGWVSIKLRKLLHKIM
DPDPSKRISIQKIKESTWERKGPEETLTMERIPIENAPTNAAPTLMRRRKNSHGDAKPLAVT
NLNAFEIISFSTGFDLSGLFVEKESKKETRFTSENPASAIISKLEDVAKALNLRVRKKDNGV
VKMQGRKEGRNGVVQFDSEIFQITPSYHLIEMKQTSGDSLEYQKLLEDGIRPALKDIVWAWH
GDDDHHQQRG
>SiCIPK15 (Setaria Italica SETIT_010116mg)
SEQ ID NO: 6
MEGRGKILMERYELGRMLGKGTFGKVHYAKNLESNQSVAIKMMDKDKVLKVGLSEQIRREIT
TMRLVAHKNIVELHEVMATRNKIYFVMEYVKGGELFEKIEKSGKLTEPAAHKYFQQLISAVD
YCHSRGVYHRDLKPENLLLDEDENLKVSDFGLSALSESKRQDGLLHTTCGTPAYVAPEVISK
IGYDGAKSDIWSCGVVLFVLVAGYLPFQGPNLMEMYRKVQHGDFRCPSWFSHKLKKLLYKIL
DPNPATRISIQKIKESTWERKGPEGTRTVKEKIPCENATTNAAPTLSVRRKKNPYEDAKPLP
VTNLNAFEIISESSGFDLSGLFLEKECRKEARFTSDKSASAIILKIEDVAKMLNLRVRKKDN
GVVKIQGRKEGRNGVLQFDTEIFEITPFHHLVEMKQTSGDFLEYQKLFEEDIRPALKDIVWA
WHGDDQQQKQ
>SevCIPK15 (Setaria viridis SEVIR_7G311940v2)
SEQ ID NO: 7
MEGRGKILMERYELGRMLGKGTFGKVHYAKNLESNQSVAIKMMDKDKVLKVGLSEQIRREIT
TMRLVAHKNIVELHEVMATRNKIYFVMEYVKGGELFEKIEKSGKLTEPAAHKYFQQLISAVD
YCHSRGVYHRDLKPENLLLDEDENLKVSDFGLSALSESKRQDGLLHTTCGTPAYVAPEVISK
IGYDGAKSDIWSCGVVLFVLVAGYLPFQGPNLMEMYRKVQHGDFRCPSWFSHKLKKLLYKIL
DPNPATRISIQKIKESTWERKGPEGTRTVKEKIPCENATTNAAPTLSVRRKKNPYEDAKPLT
VTNLNAFEIISESSGFDLSGLFLEKECRKEARFTSDKSASAIILKIEDVAKMLNLRVRKKDN
GVVKIQGRKEGRNGVLQFDTEIFEITPLHHLVEMKQTSGDFLEYQKLFEEDIRPALKDIVWA
WHGDDQQQKQ
>SbCIPK15 (Sorghum bicolor SORBI_3005G012000)
SEQ ID NO: 8
MESRGKILMKRYELGRLLGKGTFGKVHYARNLESNQSVAIKMMDKDKVLNVGLSEQIRREIT
TMRLVAHKNIVELHEVMATRNKIYFVMEYVKGGELFDKIEKSGKLTEAVAHKYFQQLISAVD
YCHSQGVYHRDLKPENLLLDENENLKVSDFGLSALSESKRQDGLLHTTCGTPAYVAPEVISK
TGYDGAKSDIWSCGVVLFVLVAGYLPFQGPNLMEMYRKIQNGNFRCPSWFSHKLKKLLYKIL
DPNPNTRISVQKIKESTWFRKGPGETRTVKEKIPSENATTNAAPVLAMRRKKNVHEDVKPLA
VTNLNAFEIISESTGFDLSGLFIKKECKKETRFTSDKPAAAIISKLEDVAKILNLRIRKMDN
GVVKIQGRKEGRNGVLQFDAEIFEITASYHLIEMKQTGGDSLEYQKLLEENIRPALKDIVWA
WHGDDQQQK
>HvCIPK15 (Hordeum vulgare HORVU.MOREX.r3.5HG0464580)
SEQ ID NO: 9
MENSGKIVMGRYELGRLLGKGAFGKVHYAKNLESNRGVAIKMMDKETVLKVGLAEQVRREIT
TMRLVAHKSIVQLHEVMATRSKIYFVMEYMKGGELFDKVSKAGKLTEGAAHKYFQQLISAVD
YCHSRGVYHRDLKPENLLLDENENLKVSDFGLSALSESKRQDGLLHTTCGSPAYVAPEVISK
GGYDGAKSDIWSCGVILFVLVAGYLPFQGQNLIEMYRKIEKGDFRCPGWVSPKLQKLLHKIM
DPDPNKRISIQKIKESTWFRKGPGENLTVKERLPNENTTTDAVPTLGVRRRKNSHEDRKPLA
VTNLNAFEIISESTGFDLSGLFIEKESKKEARFTSEQPASAIVSKLEDVAKQLNLRVRKKDN
GVVKMQVRKEGRNGVLQFDSEIFEISPSYHLIEMKQTSGDSLEYQKLLEEGIRPALKDIIWA
WHGADG
>TaCIPK15 (Triticum aestivum TraesCS4A02G194800)
SEQ ID NO: 10
MQLEVMANRGKILMERYELGRLLGKGTFGKVHYARSLESNRSVAIKMLDKEKVLKVGLSEQI
RREVTTMRLVAHKNIVQLHEVMATRNKIYFVMEYVKGGELFDKVAKSGKLTEGAAHKYFQQL
ISAVDYCHSQGVYHRDLKLENLLLDENENLKVSDFGLSALSESKRQDGLLHTTCGTPAYVAP
EVISKTGYDGAKSDIWSCGVILFVLVAGYLPFHGSNLMDMYRKIEQGDFRCPSWFSHKLQKL
LFKILDPNPSTRASIQKIKESTWFRKGPRGTLAVKERTPSENVTTNAPPTAGVRPRKNTHED
VKPLMVTNLNAFEIISFSTGFDLSGLFIREECRKETRFTSDKPASAIISKLEYVAKALNLRV
RKKDNGVVKMQARKEGRNGAVQLDMEIFEITPSHHLIEMKQTSGDPLEYRELLEDIRPALKD
IVWAWHGDDHHQQLE
>TridCIPK15 (Triticum dicoccoides TRIDC4AG031070)
SEQ ID NO: 11
MQLEVMANRGKILMERYELGRLLGKGTFGKVHYARSLESNRSVAIKMLDKEKVLKVGLSEQI
RREVTTMRLVAHKNIVQLHEVMATRNKIYFVMEYVKGGELFDKVAKSGKLTEGAAHKYFQQL
ISAVDYCHSQGVYHRDLKLENLLLDENENLKVSDFGLSALSESKRQDGLLHTTCGTPAYVAP
EVISKTGYDGAKSDIWSCGVILFVLVAGYLPFHGSNLMDMYRKIEQGDERCPSWFSHKLQKL
LFKILDPNPSTRASIQKIKESTWERKGPRGTLAVKERTPSENVTTNAPPTAGVRPRKNTHED
VKPLMVTNLNAFEIISFSTGFDLSGLFIREECRKETRFTSDKHASAIISKLEYVAKALNLRV
RKKDNGVVKMQARKEGRNGAVQLDMEIFEITPSHHLIEMKQTSGDPLEYRELLEDIRPALKD
IVWAWHGDDHQQQLE
>Tritd CIPK15 (Triticum turgidum TRITD4Av1G152750)
SEQ ID NO: 12
MQLEVMANRGKILMERYELGRLLGKGTFGKVHYARSLESNRSVAIKMLDKEKVLKVGLSEQI
RREVTTMRLVAHKNIVQLHEVMATRNKIYFVMEYVKGGELFDKVAKSGKLTEGAAHKYFQQL
ISAVDYCHSQGVYHRDLKLENLLLDENENLKVSDFGLSALSESKRQDGLLHTTCGTPAYVAP
EVISKTGYDGAKSDIWSCGVILFVLVAGYLPFHGSNLMDMYRKIEQGDERCPSWESHKLQKL
LFKILDPNPSTRASIQKIKESTWFRKGPRGTLAVKERTPSENVTTNAPPTAGVRPRKNTHED
VKPLMVTNLNAFEIISFSTGFDLSGLFIREECRKETRFTSDKPASAIISKLEYVAKALNLRV
RKKDNGVVKMQARKEGRNGAVQLDMEIFEITPSHHLIEMKQTSGDPLEYRELLEDIRPALKD
IVWAWHGDDHHQQLE
>TuGCIPk15 (Triticum urartu TuG1812G0400001280.01)
SEQ ID NO: 13
MANRGKILMERYELGRLLGKGTFGKVHYARSLESNRSVAIKMLDKEKVLKVGLSEQIRREVT
TMRLVAHKNIVQLHEVMATRNKIYFVMEYVKGGELFDKVAKSGKLTEGAAHKYFQQLISAVD
YCHSQGVYHRDLKLENLLLDENENLKVSDFGLSALSESKRQDGLLHTTCGTPAYVAPEVISK
TGYDGAKSDIWSCGVILFVLVAGYLPFHGSNLMDMYRKIEQGDFRCPSWFSHKLQKLLFKIL
DPNPSTRASIQKIKESTWFRKGPRGTLAVKERTPSENVTTNAPPTAGVRPRKNTHEDVKPLM
VTNLNAFEIISESTGFDLSGLFIQEECRKETRFTSDKPASAIISKLEYVAKALNLRVRKKDN
GVVKMQARKEGRNGAVQLDMEIFEITPSHHLIEMKQTSGDPLEYRELLEDIRPALKDIVWAW
HGDDHQQQLE
>ZmCIPK15 (Zea mays Zm00001e023967)
SEQ ID NO: 14
MESTGKILMERYEVGRLLGKGTFGKVHYAKNLESNESVAIKMMDKDKVLKAGLSEQIRREIT
TMRLVAHKNIVELHEVMATRNNIYFVMEYVKGGELFDKIGKSGKLTEAAAHKYFQQLIGAVD
YCHSRGVYHRDLKPENLLLDENENLKVSDFGLSALSESKGQDGLLHTTCGTPAYVAPEVISK
TGYDGAKSDIWSCGVVLFVLVAGYLPFRAPNMMEMYRKIQNGDFRCPSWFSHKLKKLLYKIL
DPSPDTRISVQKIKESTWFRKGPVETRTVKEKILSENDTTNAAPAVAMRRKKNVHEDVKPPT
VTSLNAFEIISFSTGLDLSGLFIRKECKKETRFTSDKGAVSIISKLEDVAKALNLRVRKKDN
GVVKIQGRKVGRKGVLEFDTEIFEITASYHLIEMKQTGGDSLEYKKLLEEDIRPALKDIVWA
WHGDDQQQK
>LpCIPK15 (Leersia perrieri LPERR11G00710)
SEQ ID NO: 15
METRGKILMERYELGRLLGKGTFGKVHYARNLESNQSVAIKMMDKQQALKVGLSEQIRREIT
TMRLVAHKNIVQLHEVMATRNKIYFVMEYVKGGELFEKVEKRGKLTEGVAHKYFQQLISAVD
YCHSRGVYHRDLKLENLLLDENENLKVSDFGLSALSESRRQDGLLHTTCGTPAYVAPEVISK
IGYDGAKSDIWSCGVILFVLVAGYLPFQGPNLMEMYRKIQHSEFRCPSWISRKLQKLLYKIM
DPNPNTRISIQKIKESTWFRKGPEENRVLKERTLNESTTRNVAPTLGVRRKKNAHEDVKPMS
VTNLNAFEIISESTGFDLSGMFIAKERRNEARFTSDKSASTIISKMEDVAKVLNLRVRKKDN
GVVKMQGRKEGRNGVLQFDMEIFEVINSYHIIEMKQTNGDPLEYRQLLEEGIRPALKDIVLA
WH
>SsCIPK15 (Saccharum spontaneum Sspon.05G0021220-2P)
SEQ ID NO: 16
MESRGKTLVERYEMGRLLGKGTFGKVHYGRNLKSNQSVAIKMMDKDKVLKVGLSEQIKREIT
TMRLVAHKNIVHLHEVMATRNKIYIVMEYVKGGELFDKIDKSGKLTEAAAHKYFQQLISAVD
HCHSRGVYHRDLKPENLLLDENENLKVSDFGLSALSESKRQDGLLHTTCGTPAYVAPEVISK
IGYDGAKSDIWSCGVVLFVLAAGYLPFQGPNLMEIYRKIQHGDFRCPSWFSHKLKKLLYKIL
DPNPSTRISIQKIKESTWERKGPGEIRAVKEKILSENATTNAAPVLATRRKKIAHEDMKPLA
ATNLNAFEIISFSTGLDLSGLFIKKECRKETRFTSDKPALAIISKLEEVAKALNLRIRKKDN
GIVKIQGRKEGRNGVLQFDTEIFEITPSYHLIEMKQTSGDSVEYQKLLEEDIRPGLKDIVWA
WHGDDLQQKQE
>PhCIPK15 (Panicum hallii HAL2 GQ55_3G008400)
SEQ ID NO: 17
MEGRGKILMERYELGRLLGKGTFGRVHYARNLESNRSVAIKMMDKDKVLKVGLSEQIRREIT
TMRLVAHKNIVELHEVMATRNKIYFVMEYVKGGELFDKIEKSGRLTESAAHNYFQQLISAVD
YCHSRGVYHRDLKPENLLLDEDENLKVSDFGLSALSESKRQDGLLHTTCGTPAYVAPEVISK
TGYDGAKSDIWSCGVVLFVLVAGYLPFQGPNLMEMYRKIQHGDFRCPSWFSHKLKKLLYKIL
DPNPSTRISIQKIKESTWFRKGPEGTRTVKEKIPSENATTNAAPTLSVRRGKNAYEDVKPLH
VTNLNAFEIISFSSGFDLSGLFIEMEHRKEARFTSDKPASAIISKIEDVAKMLNLRVRKKDN
GVVKIQGRKEGRNGVLQFDTEIFEITPFHHLVEMKQTSGDSLEYQKLFEQDIRPALKDIVWA
WHGDDQQQMQE
>ScCIPK15 (Secale cereal SECCE4Rv1G0229750)
SEQ ID NO: 18
MANRGKILMERYELGRLLGKGTFGKVHYARSLESNQSVAIKMLDKEKVLKVGLSEQIRREVT
TMRLVAHKNIVQLHEVMATRNKIYFVMEYVKGGELFDKVAKSGKLTEGVAHKYFQQLISAVD
YCHSQGVYHRDLKLENLLLDENENLKVSDFGLSALSESKRQDGLLHTTCGTPAYVAPEVISK
TGYDGAKSDIWSCGVILFVLVAGYLPFQGSNLMDMYRKIEQGDFRCPSWFSHKLQKLLCKIL
DPNPSTRASIKKIKESTWFRKGPRGTLAVKERTPSENVSTNTPPTAGVRPRKNTHEDVKPLM
VINLNAFEIISFSTGFDLSGLFIQEECRKVTRFTSDKPASAIISKLEYVAKAMNLRVRKKDN
GVVKMQARKEGRNGAVQLDIEIFEITPSHHLIEMKQTSGDPLEYRELLEDIRPRLKDIVWAW
HGDDHQQQLE
>mutant BdCIPK15 (origin, Brachypodium distachyon BRADI_4g25880v3,
with the amino acid substitution V48I)
SEQ ID NO: 19
MDSSGKVVLGRYEVGRLLGKGAFGKVHYAKNLKSNRSVAIKMLDKEKILKVGLSEQVRREIT
TMRLVAHKNIVKLHEVMATRSKIYFVMEYVKGGQLFDKISNGGKLTESAAHKYFQQLISAVD
YCHSRGVYHRDLKPENLLLDEHENLKVSDFGLSALSESKRQDGLLHTICGSPAYVAPEVISK
GGYDGAKSDIWSCGVVLFVLVAGYLPFQGQNLMEMYMKIEQGDFKCPGWVSIKLRKLLHKIM
DPDPSKRISIQKIKESTWFRKGPEETLTMERIPIENAPTNAAPTLMRRRKNSHGDAKPLAVT
NLNAFEIISFSTGFDLSGLFVEKESKKETRFTSENPASAIISKLEDVAKALNLRVRKKDNGV
VKMQGRKEGRNGVVQFDSEIFQITPSYHLIEMKQTSGDSLEYQKLLEDGIRPALKDIVWAWH
GDDDHHQQRG
>mutant SiCIPK15 (origin, Setaria Italica SETIT_010116mg, with the
amino acid substitution V48I)
SEQ ID NO: 20
MEGRGKILMERYELGRMLGKGTFGKVHYAKNLESNQSVAIKMMDKDKILKVGLSEQIRREIT
TMRLVAHKNIVELHEVMATRNKIYFVMEYVKGGELFEKIEKSGKLTEPAAHKYFQQLISAVD
YCHSRGVYHRDLKPENLLLDEDENLKVSDFGLSALSESKRQDGLLHTTCGTPAYVAPEVISK
IGYDGAKSDIWSCGVVLFVLVAGYLPFQGPNLMEMYRKVQHGDFRCPSWFSHKLKKLLYKIL
DPNPATRISIQKIKESTWFRKGPEGTRTVKEKIPCENATTNAAPTLSVRRKKNPYEDAKPLP
VTNLNAFEIISFSSGFDLSGLFLEKECRKEARFTSDKSASAIILKIEDVAKMLNLRVRKKDN
GVVKIQGRKEGRNGVLQFDTEIFEITPFHHLVEMKQTSGDFLEYQKLFEEDIRPALKDIVWA
WHGDDQQQKQ
>mutant SevCIPK15 (origin, Setaria viridis SEVIR_7G311940v2, with
the amino acid substitution V48I)
SEQ ID NO: 21
MEGRGKILMERYELGRMLGKGTFGKVHYAKNLESNQSVAIKMMDKDKILKVGLSEQIRREIT
TMRLVAHKNIVELHEVMATRNKIYFVMEYVKGGELFEKIEKSGKLTEPAAHKYFQQLISAVD
YCHSRGVYHRDLKPENLLLDEDENLKVSDFGLSALSESKRQDGLLHTTCGTPAYVAPEVISK
IGYDGAKSDIWSCGVVLFVLVAGYLPFQGPNLMEMYRKVQHGDFRCPSWFSHKLKKLLYKIL
DPNPATRISIQKIKESTWERKGPEGTRTVKEKIPCENATTNAAPTLSVRRKKNPYEDAKPLT
VTNLNAFEIISESSGFDLSGLFLEKECRKEARFTSDKSASAIILKIEDVAKMLNLRVRKKDN
GVVKIQGRKEGRNGVLQFDTEIFEITPLHHLVEMKQTSGDFLEYQKLFEEDIRPALKDIVWA
WHGDDQQQKQ
>mutant SbCIPK15 (origin, Sorghum bicolor SORBI_3005G012000, with
the amino acid substitution V48I)
SEQ ID NO: 22
MESRGKILMKRYELGRLLGKGTFGKVHYARNLESNQSVAIKMMDKDKILNVGLSEQIRREIT
TMRLVAHKNIVELHEVMATRNKIYFVMEYVKGGELFDKIEKSGKLTEAVAHKYFQQLISAVD
YCHSQGVYHRDLKPENLLLDENENLKVSDFGLSALSESKRQDGLLHTTCGTPAYVAPEVISK
TGYDGAKSDIWSCGVVLFVLVAGYLPFQGPNLMEMYRKIQNGNFRCPSWFSHKLKKLLYKIL
DPNPNTRISVQKIKESTWFRKGPGETRTVKEKIPSENATTNAAPVLAMRRKKNVHEDVKPLA
VTNLNAFEIISESTGFDLSGLFIKKECKKETRFTSDKPAAAIISKLEDVAKILNLRIRKMDN
GVVKIQGRKEGRNGVLQFDAEIFEITASYHLIEMKQTGGDSLEYQKLLEENIRPALKDIVWA
WHGDDQQQK
>mutant HvCIPK15 (origin, Hordeum vulgare HORVU.MOREX.r3.5HG0464580,
with the amino acid substitution V48I)
SEQ ID NO: 23
MENSGKIVMGRYELGRLLGKGAFGKVHYAKNLESNRGVAIKMMDKETILKVGLAEQVRREIT
TMRLVAHKSIVQLHEVMATRSKIYFVMEYMKGGELFDKVSKAGKLTEGAAHKYFQQLISAVD
YCHSRGVYHRDLKPENLLLDENENLKVSDFGLSALSESKRQDGLLHTTCGSPAYVAPEVISK
GGYDGAKSDIWSCGVILFVLVAGYLPFQGQNLIEMYRKIEKGDFRCPGWVSPKLQKLLHKIM
DPDPNKRISIQKIKESTWFRKGPGENLTVKERLPNENTTTDAVPTLGVRRRKNSHEDRKPLA
VTNLNAFEIISFSTGFDLSGLFIEKESKKEARFTSEQPASAIVSKLEDVAKQLNLRVRKKDN
GVVKMQVRKEGRNGVLQFDSEIFEISPSYHLIEMKQTSGDSLEYQKLLEEGIRPALKDIIWA
WHGADG
>mutant TaCIPK15 (origin, Triticum aestivum TraesCS4A02G194800,
with the amino acid substitution V53I)
SEQ ID NO: 24
MQLEVMANRGKILMERYELGRLLGKGTFGKVHYARSLESNRSVAIKMLDKEKILKVGLSEQI
RREVTTMRLVAHKNIVQLHEVMATRNKIYFVMEYVKGGELEDKVAKSGKLTEGAAHKYFQQL
ISAVDYCHSQGVYHRDLKLENLLLDENENLKVSDFGLSALSESKRQDGLLHTTCGTPAYVAP
EVISKTGYDGAKSDIWSCGVILFVLVAGYLPFHGSNLMDMYRKIEQGDFRCPSWFSHKLQKL
LFKILDPNPSTRASIQKIKESTWFRKGPRGTLAVKERTPSENVTTNAPPTAGVRPRKNTHED
VKPLMVTNLNAFEIISFSTGFDLSGLFIREECRKETRFTSDKPASAIISKLEYVAKALNLRV
RKKDNGVVKMQARKEGRNGAVQLDMEIFEITPSHHLIEMKQTSGDPLEYRELLEDIRPALKD
IVWAWHGDDHHQQLE
>mutant TridCIPK15 (origin, Triticum dicoccoides TRIDC4AG031070,
with the amino acid substitution V53I)
SEQ ID NO: 25
MQLEVMANRGKILMERYELGRLLGKGTFGKVHYARSLESNRSVAIKMLDKEKILKVGLSEQI
RREVTTMRLVAHKNIVQLHEVMATRNKIYFVMEYVKGGELFDKVAKSGKLTEGAAHKYFQQL
ISAVDYCHSQGVYHRDLKLENLLLDENENLKVSDFGLSALSESKRQDGLLHTTCGTPAYVAP
EVISKTGYDGAKSDIWSCGVILFVLVAGYLPFHGSNLMDMYRKIEQGDFRCPSWFSHKLQKL
LFKILDPNPSTRASIQKIKESTWFRKGPRGTLAVKERTPSENVTTNAPPTAGVRPRKNTHED
VKPLMVTNLNAFEIISFSTGFDLSGLFIREECRKETRFTSDKHASAIISKLEYVAKALNLRV
RKKDNGVVKMQARKEGRNGAVQLDMEIFEITPSHHLIEMKQTSGDPLEYRELLEDIRPALKD
IVWAWHGDDHQQQLE
>mutant TritdCIPK15 (origin, Triticum turgidum TRITD4Av1G152750,
with the amino acid substitution V53I)
SEQ ID NO: 26
MQLEVMANRGKILMERYELGRLLGKGTFGKVHYARSLESNRSVAIKMLDKEKILKVGLSEQI
RREVTTMRLVAHKNIVQLHEVMATRNKIYFVMEYVKGGELFDKVAKSGKLTEGAAHKYFQQL
ISAVDYCHSQGVYHRDLKLENLLLDENENLKVSDFGLSALSESKRQDGLLHTTCGTPAYVAP
EVISKTGYDGAKSDIWSCGVILFVLVAGYLPFHGSNLMDMYRKIEQGDFRCPSWFSHKLQKL
LFKILDPNPSTRASIQKIKESTWERKGPRGTLAVKERTPSENVTTNAPPTAGVRPRKNTHED
VKPLMVTNLNAFEIISFSTGFDLSGLFIREECRKETRFTSDKPASAIISKLEYVAKALNLRV
RKKDNGVVKMQARKEGRNGAVQLDMEIFEITPSHHLIEMKQTSGDPLEYRELLEDIRPALKD
IVWAWHGDDHHQQLE
>mutant TuGCIPk15 (origin, Triticum urartu TuG1812G0400001280.01,
with the amino acid substitution V48I)
SEQ ID NO: 27
MANRGKILMERYELGRLLGKGTFGKVHYARSLESNRSVAIKMLDKEKILKVGLSEQIRREVT
TMRLVAHKNIVQLHEVMATRNKIYFVMEYVKGGELFDKVAKSGKLTEGAAHKYFQQLISAVD
YCHSQGVYHRDLKLENLLLDENENLKVSDFGLSALSESKRQDGLLHTTCGTPAYVAPEVISK
TGYDGAKSDIWSCGVILFVLVAGYLPFHGSNLMDMYRKIEQGDFRCPSWFSHKLQKLLFKIL
DPNPSTRASIQKIKESTWFRKGPRGTLAVKERTPSENVTTNAPPTAGVRPRKNTHEDVKPLM
VTNLNAFEIISFSTGFDLSGLFIQEECRKETRFTSDKPASAIISKLEYVAKALNLRVRKKDN
GVVKMQARKEGRNGAVQLDMEIFEITPSHHLIEMKQTSGDPLEYRELLEDIRPALKDIVWAW
HGDDHQQQLE
>mutant ZmCIPK15 (origin, Zea mays Zm00001e023967, with the amino
acid substitution V48I)
SEQ ID NO: 28
MESTGKILMERYEVGRLLGKGTFGKVHYAKNLESNESVAIKMMDKDKILKAGLSEQIRREIT
TMRLVAHKNIVELHEVMATRNNIYFVMEYVKGGELFDKIGKSGKLTEAAAHKYFQQLIGAVD
YCHSRGVYHRDLKPENLLLDENENLKVSDFGLSALSESKGQDGLLHTTCGTPAYVAPEVISK
TGYDGAKSDIWSCGVVLFVLVAGYLPFRAPNMMEMYRKIQNGDFRCPSWFSHKLKKLLYKIL
DPSPDTRISVQKIKESTWFRKGPVETRTVKEKILSENDTTNAAPAVAMRRKKNVHEDVKPPT
VTSLNAFEIISESTGLDLSGLFIRKECKKETRFTSDKGAVSIISKLEDVAKALNLRVRKKDN
GVVKIQGRKVGRKGVLEFDTEIFEITASYHLIEMKQTGGDSLEYKKLLEEDIRPALKDIVWA
WHGDDQQQK
>mutant LpCIPK15 (origin, Leersia perrieri LPERR11G00710, with the
amino acid substitution A48I)
SEQ ID NO: 29
METRGKILMERYELGRLLGKGTFGKVHYARNLESNQSVAIKMMDKQQILKVGLSEQIRREIT
TMRLVAHKNIVQLHEVMATRNKIYFVMEYVKGGELFEKVEKRGKLTEGVAHKYFQQLISAVD
YCHSRGVYHRDLKLENLLLDENENLKVSDFGLSALSESRRQDGLLHTTCGTPAYVAPEVISK
IGYDGAKSDIWSCGVILFVLVAGYLPFQGPNLMEMYRKIQHSEFRCPSWISRKLQKLLYKIM
DPNPNTRISIQKIKESTWERKGPEENRVLKERTLNESTTRNVAPTLGVRRKKNAHEDVKPMS
VTNLNAFEIISESTGFDLSGMFIAKERRNEARFTSDKSASTIISKMEDVAKVLNLRVRKKDN
GVVKMQGRKEGRNGVLQFDMEIFEVTNSYHIIEMKQTNGDPLEYRQLLEEGIRPALKDIVLA
WH
>mutant SsCIPK15 (origin, Saccharum spontaneum Sspon.05G0021220-2P,
with the amino acid substitution V48I)
SEQ ID NO: 30
MESRGKTLVERYEMGRLLGKGTFGKVHYGRNLKSNQSVAIKMMDKDKILKVGLSEQIKREIT
TMRLVAHKNIVHLHEVMATRNKIYIVMEYVKGGELFDKIDKSGKLTEAAAHKYFQQLISAVD
HCHSRGVYHRDLKPENLLLDENENLKVSDFGLSALSESKRQDGLLHTTCGTPAYVAPEVISK
IGYDGAKSDIWSCGVVLFVLAAGYLPFQGPNLMEIYRKIQHGDFRCPSWFSHKLKKLLYKIL
DPNPSTRISIQKIKESTWFRKGPGEIRAVKEKILSENATTNAAPVLATRRKKIAHEDMKPLA
ATNLNAFEIISESTGLDLSGLFIKKECRKETRFTSDKPALAIISKLEEVAKALNLRIRKKDN
GIVKIQGRKEGRNGVLQFDTEIFEITPSYHLIEMKQTSGDSVEYQKLLEEDIRPGLKDIVWA
WHGDDLQQKQE
>mutant PhCIPK15 (origin, Panicum hallii HAL2 GQ55_3G008400, with
the amino acid substitution V481)
SEQ ID NO: 31
MEGRGKILMERYELGRLLGKGTFGRVHYARNLESNRSVAIKMMDKDKILKVGLSEQIRREIT
TMRLVAHKNIVELHEVMATRNKIYFVMEYVKGGELFDKIEKSGRLTESAAHNYFQQLISAVD
YCHSRGVYHRDLKPENLLLDEDENLKVSDFGLSALSESKRQDGLLHTTCGTPAYVAPEVISK
TGYDGAKSDIWSCGVVLFVLVAGYLPFQGPNLMEMYRKIQHGDFRCPSWFSHKLKKLLYKIL
DPNPSTRISIQKIKESTWFRKGPEGTRTVKEKIPSENATTNAAPTLSVRRGKNAYEDVKPLH
VTNLNAFEIISESSGFDLSGLFIEMEHRKEARFTSDKPASAIISKIEDVAKMLNLRVRKKDN
GVVKIQGRKEGRNGVLQFDTEIFEITPFHHLVEMKQTSGDSLEYQKLFEQDIRPALKDIVWA
WHGDDQQQMQE
>mutant ScCIPK15 (origin, Secale cereal SECCE4Rv1G0229750, with the
amino acid substitution V48I)
SEQ ID NO: 32
MANRGKILMERYELGRLLGKGTFGKVHYARSLESNQSVAIKMLDKEKILKVGLSEQIRREVT
TMRLVAHKNIVQLHEVMATRNKIYFVMEYVKGGELFDKVAKSGKLTEGVAHKYFQQLISAVD
YCHSQGVYHRDLKLENLLLDENENLKVSDFGLSALSESKRQDGLLHTTCGTPAYVAPEVISK
TGYDGAKSDIWSCGVILFVLVAGYLPFQGSNLMDMYRKIEQGDFRCPSWFSHKLQKLLCKIL
DPNPSTRASIKKIKESTWFRKGPRGTLAVKERTPSENVSTNTPPTAGVRPRKNTHEDVKPLM
VTNLNAFEIISFSTGFDLSGLFIQEECRKVTRFTSDKPASAIISKLEYVAKAMNLRVRKKDN
GVVKMQARKEGRNGAVQLDIEIFEITPSHHLIEMKQTSGDPLEYRELLEDIRPRLKDIVWAW
HGDDHQQQLE
>OsCIPK14 (Oryza sativa Os11g0113700)
SEQ ID NO: 33
MESRGKILMERYELGRLLGKGTFGKVHYARNLESNQSVAIKMMDKQQVLKVGLSEQIRREIT
TMRLVAHKNIVQLHEVMATRNKIYFVMEYVKGGELFEKVAKRGKLTEVVAHKYFQQLISAVD
YCHSRGVYHRDLKPENLLLDENENLKVSDFGLSALSESKRQDGLLHTTCGTPAYVAPEVISK
IGYDGAKSDIWSCGVILFVLVAGYLPFQGPNLMEMYRKIQHGEFRCPGWFSRKLQKLLYKIM
DPNPSTRISIQKIKESTWERKGPEENRILKERTLNENTTKNVALVLGVRRKKNAHEDVKPMS
VTNLNAFEIISFSKGFDLSGMFIVKEWRNEARFTSDKSASTIISKLEDVAKALNLRVRKKDN
GVVKMQGRKEGRNGVLQFDIEIFEVTTSYHIIEMKQTSGDSLEYRQLLEEGIRPALKDIVLA
WHGDE

### REFERENCE

1 Bailey-Serres, J., Lee, S. C. & Brinton, E. Waterproofing crops: effective flooding survival strategies. Plant Physiol 160, 1698-1709, doi:10.1104/pp. 112.208173 (2012).
2 Lee, K. W. et al. Coordinated responses to oxygen and sugar deficiency allow rice seedlings to tolerate flooding. Sci Signal 2, ra61, doi:10.1126/scisignal.2000333 (2009).
Das, A. & Uchimiya, H. Oxygen stress and adaptation of a semi-aquatic plant: rice (Oryza sativa). J Plant Res 115, 315-320, doi:10.1007/s10265-002-0043-9 (2002).
4 Colmer, T. D. Aerenchyma and an inducible barrier to radial oxygen loss facilitate root aeration in upland, paddy and deep-water rice (Oryza sativa L.). Ann Bot (Lond) 91 Spec No, 301-309 (2003).
5. Fukao, T. & Xiong, L. Genetic mechanisms conferring adaptation to submergence and drought in rice: simple or complex? Curr Opin Plant Biol 16, 196-204, doi:10.1016/j.pbi.2013.02.003 (2013).
6 Miro, B. & Ismail, A. M. Tolerance of anaerobic conditions caused by flooding during germination and early growth in rice (Oryza sativa L.). Frontiers in plant science 4, 269, doi:10.3389/fpls.2013.00269 (2013).
7 Lee, K. W., Chen, P. W. & Yu, S. M. Metabolic adaptation to sugar/O₂ deficiency for anaerobic germination and seedling growth in rice. Plant Cell Environ 37, 2234-2244, doi:10.1111/pce.12311 (2014).
8 Yu, S. M., Lee, H. T., Lo, S. F. & Ho, T. D. How does rice cope with too little oxygen during its early life? New Phytol 229, 36-41, doi:10.1111/nph.16395 (2021).
9 Ismail, A. M., Ella, E. S., Vergara, G. V. & Mackill, D. J. Mechanisms associated with tolerance to flooding during germination and early seedling growth in rice (Oryza sativa). Ann Bot 103, 197-209, doi:10.1093/aob/mcn211 (2009).
10. Guglielminetti, L., Yamaguchi, J., Perata, P. & Alpi, A. Amylolytic activities in cereal seeds under aerobic and anaerobic conditions. Plant Physiol 109, 1069-1076 (1995).
11 Lu, C. A., Ho, T. H., Ho, S. L. & Yu, S. M. Three novel MYB proteins with one DNA binding repeat mediate sugar and hormone regulation of alpha-amylase gene expression. Plant Cell 14, 1963-1980 (2002).
12 Lu, C. A., Lim, E. K. & Yu, S. M. Sugar response sequence in the promoter of a rice alpha-amylase gene serves as a transcriptional enhancer. J Biol Chem 273, 10120-10131 (1998).
13 Lu, C. A. et al. The SnRK1A protein kinase plays a key role in sugar signaling during germination and seedling growth of rice. Plant Cell 19, 2484-2499 (2007).
14 Kawai, M. & Uchimiya, H. Coleoptile senescence in rice (Oryza sativa L.). Ann Bot 86, 405-414, doi:DOI 10.1006/anbo.2000.1199 (2000).
15 Yamauchi, T., Colmer, T. D., Pedersen, O. & Nakazono, M. Regulation of root traits for internal aeration and tolerance to soil waterlogging-flooding stress. Plant Physiol 176, 1118-1130, doi:10.1104/pp. 17.01157 (2018).
16 Colmer, T. D. & Pedersen, O. Oxygen dynamics in submerged rice (Oryza sativa). New Phytol 178, 326-334, doi:10.1111/j.1469-8137.2007.02364.x (2008).
17 Alpi, A. & Beevers, H. Effects of 02 Concentration on Rice Seedlings. Plant Physiol 71, 30-34 (1983).
18 Drew, M. C., He, C. J. & Morgan, P. W. Programmed cell death and aerenchyma formation in roots. Trends Plant Sci 5, 123-127, doi:10.1016/s1360-1385(00)01570-3 (2000).
19 Yamauchi, T. et al. Ethylene-dependent aerenchyma formation in adventitious roots is regulated differently in rice and maize. Plant Cell Environ 39, 2145-2157, doi:10.1111/pce.12766 (2016).
20 Rajhi, I. et al. Identification of genes expressed in maize root cortical cells during lysigenous aerenchyma formation using laser microdissection and microarray analyses. New Phytol 190, 351-368, doi:10.1111/j.1469-8137.2010.03535.x (2011).
21 Yoo, Y. H., Choi, H. K. & Jung, K. H. Genome-wide identification and analysis of genes associated with lysigenous aerenchym formation in rice roots. J. Plant Biol 58:117-127 (2015).
22 Cho, Y. H., Yoo, S. D. & Sheen, J. Regulatory functions of nuclear hexokinasel complex in glucose signaling. Cell 127, 579-589, doi:10.1016/j.cell.2006.09.028 (2006).
23 Moore, B. et al. Role of the Arabidopsis glucose sensor HXK1 in nutrient, light, and hormonal signaling. Science 300, 332-336, doi:10.1126/science.1080585 (2003).
24 Frommer, W. B., Schulze, W. X. & Lalonde, S. Plant science. Hexokinase, Jack-of-all-trades. Science 300, 261-263, doi:10.1126/science.1084120 (2003).
25 Cho, J. I. et al. Structure, expression, and functional analysis of the hexokinase gene family in rice (Oryza sativa L.). Planta 224, 598-611, doi:10.1007/s00425-006-0251-y (2006).
26 Karve, R. et al. Evolutionary lineages and functional diversification of plant hexokinases. Mol Plant 3, 334-346, doi:10.1093/mp/ssq003 (2010).
27 Aguilera-Alvarado, G. P. & Sanchez-Nieto, S. Plant Hexokinases are Multifaceted Proteins. Plant Cell Physiol 58, 1151-1160, doi:10.1093/pcp/pcx062 (2017).
28 Shoshan-Barmatz, V. & Mizrachi, D. VDAC1: from structure to cancer therapy. Front Oncol 2, 164, doi:10.3389/fonc.2012.00164 (2012).
29 Pastorino, J. G. & Hoek, J. B. Regulation of hexokinase binding to VDAC. J Bioenerg Biomembr 40, 171-182, doi:10.1007/s10863-008-9148-8 (2008).
30 Lampropoulos, A. et al. GreenGate—a novel, versatile, and efficient cloning system for plant transgenesis. PLoS One 8, e83043, doi:10.1371/journal.pone.0083043 (2013).
31 Chen, Y. S. et al. Sugar starvation-regulated MYBS2 and 14-3-3 protein interactions enhance plant growth, stress tolerance, and grain weight in rice. Proc Natl Acad Sci USA 116, 21925-21935, doi:10.1073/pnas.1904818116 (2019).
32 Lin, C. R. et al. SnRK1A-interacting negative regulators modulate the nutrient starvation signaling sensor SnRK1 in source-sink communication in cereal seedlings under abiotic stress. Plant Cell 26, 808-827, doi:10.1105/tpc.113.121939 (2014).
33 Chen, Y. S. et al. A late embryogenesis abundant protein HVA1 regulated by an inducible promoter enhances root growth and abiotic stress tolerance in rice without yield penalty. Plant Biotechnol J 13, 105-116, doi:10.1111/pbi.12241 (2015).
34 Ho, S. L., Tong, W. F. & Yu, S. M. Multiple mode regulation of a cysteine proteinase gene expression in rice. Plant Physiol 122, 57-66. (2000).
35 Zhang, Y. et al. A highly efficient rice green tissue protoplast system for transient gene expression and studying light/chloroplast-related processes. Plant methods 7, 30, doi:1746-4811-7-30 (2011).
36 Zuo, J., Hare, P. D. & Chua, N. H. Applications of chemical-inducible expression systems in functional genomics and biotechnology. Methods in molecular biology 323, 329-342, doi:10.1385/1-59745-003-0:329 (2006).
37 Yang, C., Lu, X., Ma, B., Chen, S. Y. & Zhang, J. S. Ethylene signaling in rice and Arabidopsis: conserved and diverged aspects. Mol Plant 8, 495-505, doi:10.1016/j.molp.2015.01.003 (2015).
38 Yang, S. F. & Hoffman, N. E. Ethylene Biosynthesis and Its Regulation in Higher-Plants. Annu Rev Plant Phys 35, 155-189, doi:DOI 10.1146/annurev.pp. 35.060184.001103 (1984).
39 Cho, J. I. et al. Role of the rice hexokinases OsHXK5 and OsHXK6 as glucose sensors. Plant Physiol 149, 745-759, doi:10.1104/pp. 108.131227 (2009).
He, C. et al. Crystal structures of rice hexokinase 6 with a series of substrates shed light on its enzymatic mechanism. Biochem Biophys Res Commun 515, 614-620, doi:10.1016/j.bbrc.2019.05.139 (2019).
41 Ma, X. et al. The CBL-CIPK Pathway in Plant Response to Stress Signals. International journal of molecular sciences 21, doi:10.3390/ijms21165668 (2020).
42 Sasidharan, R. et al. Signal dynamics and interactions during flooding stress. Plant Physiol 176, 1106-1117, doi:10.1104/pp. 17.01232 (2018).
43 Yamauchi, T. et al. Ethylene Biosynthesis Is Promoted by Very-Long-Chain Fatty Acids during Lysigenous Aerenchyma Formation in Rice Roots. Plant Physiol 169, 180-193, doi:10.1104/pp. 15.00106 (2015).
44 Lynch, J. P. Root phenes that reduce the metabolic costs of soil exploration: opportunities for 21st century agriculture. Plant Cell Environ 38, 1775-1784, doi:10.1111/pce.12451 (2015).
45 Clark, L. J., Price, A. H., Steele, K. A. & Whalley, W. R. Evidence from near-isogenic lines that root penetration increases with root diameter and bending stiffness in rice. Funct Plant Biol 35, 1163-1171, doi:10.1071/FP08132 (2008).
46 Tang, R. J., Wang, C., Li, K. & Luan, S. The CBL-CIPK Calcium Signaling Network: Unified Paradigm from 20 Years of Discoveries. Trends Plant Sci 25, 604-617, doi:10.1016/j.tplants.2020.01.009 (2020).
47 Guo, Y., Halfter, U., Ishitani, M. & Zhu, J. K. Molecular characterization of functional domains in the protein kinase SOS2 that is required for plant salt tolerance. Plant Cell 13, 1383-1400, doi:10.1105/tpc.13.6.1383 (2001).
48 Lee, S. K. et al. Deficiency of rice hexokinase HXK5 impairs synthesis and utilization of starch in pollen grains and causes male sterility. J Exp Bot 71, 116-125, doi:10.1093/jxb/erz436 (2020).
49 Sebastian, J. & Dinneny, J. R. in Genetics and Genomics of Setaria (eds Andrew Doust & Xianmin Diao) 177-193 (Springer International Publishing, 2017).
50 Ortiz-Ramirez, C. et al. Ground tissue circuitry regulates organ complexity in maize and Setaria. Science 374, 1247-1252, doi:10.1126/science.abj2327 (2021).
51 Matsuura, A., Kato, Y., Suzuki, T., Murata, K. & an, P. Hypoxia tolerance of four millet species is attributable to constitutive aerenchyma formation and root hair development of adventitious roots. Plant Production Science 25, 157-171, doi:10.1080/1343943X.2021.2021092 (2022).
52 Matsuura, A., An, P., Murata, K. & Inanaga, S. Effect of pre- and post-heading waterlogging on growth and grain yield of four millets. Plant Production Science 19, 348-359, doi:10.1080/1343943X.2016.1146907 (2016).
53 Sasidharan, R. & Voesenek, L. A. Ethylene-Mediated Acclimations to Flooding Stress. Plant physiology 169, 3-12 (2015).
54. S. Rocha et al., Lignification of developing maize (Zea mays L.) endosperm transfer cells and starchy endosperm cells. Frontiers in plant science 5, 102 (2014).
55. M. C. Brundrett, B. Kendrick, C. A. Peterson, Efficient lipid staining in plant material with sudan red 7B or fluorol [correction of fluoral] yellow 088 in polyethylene glycol-glycerol. Biotech Histochem 66, 111-116 (1991).
56. R. Landgraf et al., The ABC transporter ABCG1 is required for suberin formation in potato tuber periderm. Plant Cell 26, 3403-3415 (2014).

What is claimed is:

1. A method for promoting growth, yield and/or root development of a plant, comprising
(a) transforming plant cells with a recombinant construct comprising a nucleic acid operably linked to a promoter to obtain recombinant plant cells, wherein the nucleic acid encodes a calcineurin B-like (CBL) interacting protein kinase15 (CIPK15) having an isoleucine (Ile) residue at position 48 of SEQ ID NO: 1 and the recombinant plant cells overexpressing the CIPK15;
(b) growing the recombinant plant cells obtained in (a) to generate a plurality of transgenic plants; and
(c) selecting a transgenic plant from the plurality of transgenic plants generated in (b) that exhibits improved growth, yield and/or root development as compared with a non-transgenic plant counterpart growing under the same conditions.

2. The method of claim 1, wherein the CIPK15 is a CIPK15 from rice.

3. The method of claim 1, wherein the CIPK15 is a CIPK15 from barley, wheat, rye, oat, millet, corn, bamboo, sugarcane, onion, leek, ginger, stiff brome, millet, green foxtail, sorghum, Kans grass or Hall's panicgrass, having an isoleucine (Ile) substituent at a position corresponding to position 48 of SEQ ID NO: 1.

4. The method of claim 1, wherein the CIPK15 comprises
(a) an amino acid sequence selected from the group consisting of SEQ ID NOs: 1-4 or an amino acid sequence selected from the group consisting of SEQ ID NOs: 19-32; or
(b) an amino acid sequence having a sequence identity of at least 80% with the amino acid sequence of (a) and having an isoleucine (Ile) residue at position 48 of SEQ ID NO: 1.

5. The method of claim 1, wherein the promoter is a root-specific promoter.

6. The method of claim 1, wherein the promoter is a native rice CIPK15 promoter.

7. The method of claim 1, wherein the transgenic plant exhibits improved growth, yield and/or root development as compared with a non-transgenic plant counterpart under aerobic or hypoxic conditions.

8. The method of claim 1, wherein the transgenic plant exhibits increased plant height, elongated coleoptiles and shoot, higher grain yield, longer roots, larger root radius, and/or more aerenchyma.

9. The method of claim 1, wherein the transgenic plant further exhibits enhanced tolerance to environmental stress.

10. The method of claim 9, wherein the environmental stress includes drought, salinity and/or flooding stresses.

11. The method of claim 1, wherein the transgenic plant further exhibits enhanced ethylene production.

12. The method of claim 1, wherein the transgenic plant is a monocot plant.

13. The method of claim 12, wherein the monocot plant is rice, barley, wheat, rye, oat, corn, bamboo, sugarcane, onion, leek, ginger, stiff brome, millet, green foxtail, sorghum, cutgrasses, Kans grass or Hall's panicgrass.

14. The method of claim 1, wherein, the transgenic plant is a dicot plant.

15. The method of claim 14, wherein the transgenic plant is Arabidopsis, soybean, peanut, sunflower, safflower, cotton, tobacco, tomato, pea, chickpea, pigeon pea or potato.

16. A recombinant construct comprising a nucleic acid encoding a calcineurin B-like (CBL) interacting protein kinase15 (CIPK15) operably linked to a promoter, wherein the CIPK15 has an isoleucine (Ile) residue at a position corresponding to position 48 of SEQ ID NO: 1.

17. The recombinant construct of claim 16, wherein the CIPK15 is a CIPK15 from rice.

18. The recombinant construct of claim 16, wherein the CIPK15 is a CIPK15 from barley, wheat, rye, oat, millet, corn, bamboo, sugarcane, onion, leek, ginger, stiff brome, millet, green foxtail, sorghum, Kans grass or Hall's panicgrass, having an isoleucine (Ile) substituent at a position corresponding to position 48 of SEQ ID NO: 1.

19. The recombinant construct of claim 16, wherein the CIPK15 comprises
(a) an amino acid sequence selected from the group consisting of SEQ ID NOs: 1-4 or an amino acid sequence selected from the group consisting of SEQ ID NOs:19-32; or
(b) an amino acid sequence having a sequence identity of at least 80% with the amino acid sequence of (a) and having an isoleucine (Ile) residue at position 48 of SEQ ID NO: 1.

20. The recombinant construct of claim 16, wherein the promoter is a root-specific promoter.

21. The recombinant construct of claim 16, wherein the promoter is a native rice CIPK15 promoter.

22. The recombinant construct of claim 16, wherein the recombinant construct is a vector.

23. A transgenic plant comprising an exogenous nucleic acid operably linked to a promoter, wherein the exogenous nucleic acid encodes a CIPK15.

24. The transgenic plant of claim 23, wherein the transgenic plant exhibits improved growth, yield and/or root development as compared with a non-transgenic plant counterpart.

25. The transgenic plant of claim 23, wherein the transgenic plant exhibits improved growth, yield and/or root development as compared with a non-transgenic plant counterpart under aerobic or hypoxic conditions.

26. The transgenic plant of claim 23, wherein the transgenic plant exhibits increased plant height, elongated coleoptiles and shoot, higher grain yield, longer roots, larger root radius, and/or more aerenchyma.

27. The transgenic plant of claim 23, wherein the transgenic plant further exhibits enhanced tolerance to environmental stress.

28. The transgenic plant of claim 27, wherein the environmental stress includes drought, salinity and/or flooding stresses.

29. The transgenic plant of claim 23, wherein the transgenic plant further exhibits enhanced ethylene production.

30. The transgenic plant of claim 23, wherein the transgenic plant is a monocot plant.

31. The transgenic plant of claim 30, wherein the monocot plant is rice, barley, wheat, rye, oat, corn, bamboo, sugarcane, onion, leek, ginger, stiff brome, millet, green foxtail, sorghum, cutgrasses, Kans grass or Hall's panicgrass.

32. The transgenic plant of claim 23, wherein the transgenic plant is a dicot plant.

33. The transgenic plant of claim 32, wherein the transgenic plant is Arabidopsis, soybean, peanut, sunflower, safflower, cotton, tobacco, tomato, pea, chickpea, pigeon pea or potato.

34. The transgenic plant of claim 23, wherein the CIPK15 has an isoleucine (Ile) residue at a position corresponding to position 48 of SEQ ID NO: 1.

35. The transgenic plant of claim 23, wherein
the CIPK15 is a CIPK15 from rice; or
the CIPK15 is a CIPK15 from barley, wheat, rye, oat, millet, corn, bamboo, sugarcane, onion, leek, ginger, stiff brome, millet, green foxtail, sorghum, Kans grass or Hall's panicgrass, having an isoleucine (Ile) substituent at a position corresponding to position 48 of SEQ ID NO: 1.

36. The transgenic plant of claim 23, wherein the CIPK15 comprises
(a) an amino acid sequence selected from the group consisting of SEQ ID NOs: 1-4 or an amino acid sequence selected from the group consisting of SEQ ID NOs:19-32; or
(b) an amino acid sequence having a sequence identity of at least 80% with the amino acid sequence of (a).

37. A gene-edited plant having a genome comprising a nucleic acid encoding a CIPK15 having an isoleucine (Ile) substituent at a position corresponding to position 48 of SEQ ID NO: 1.

38. The gene-edited plant of claim 37, wherein the CIPK15 comprises
(a) an amino acid sequence selected from the group consisting of SEQ ID NOs:19-32; or
(b) an amino acid sequence having a sequence identity of at least 80% with the amino acid sequence of (a) and having an isoleucine (Ile) residue at position 48 of SEQ ID NO: 1.


SEQUENCE LISTING (39 sequences)

SEQ ID NO: 1 - protein, 434 aa, Oryza sativa
MESRGKILMERYELGRLLGKGTFGKVHYARNLESNQSVAIKMMDKQQILKVGLSEQIRREITTMRLVAHKNIVQLHEVMATRNKIYFVMEYVKGGELFEKVAKRGKLTEVVAHKYFQQLISAVDYCHSRGVYHRDLKPENLLLDENENLKVSDFGLSALSESKRQDGLLHTTCGTPAYVAPEVISKIGYDGAKSDIWSCGVILFVLVAGYLPFQGPNLMEMYRKIQHGEFRCPGWFSRKLQKLLYKIMDPNPSTRISIQKIKESTWFRKGPEENRILKERTLNENTTKNVAPVLGVRRKKNAHEDVKPMSVTNLNAFEIISFSKGFDLSGMFIVKEWRNEARFTSDKSASTIISKLEDVAKALNLRVRKKDNGVVKMQGRKEGRNGVLQFDIEIFEVTTSYHIIEMKQTSGDSLEYRQLLEEGIRPALKDIVLA

SEQ ID NO: 2 - protein, 434 aa, Oryza barthii
MESRGKILMERYELGRLLGKGTFGKVHYARNLESNQSVAIKMMDKQQILKVGLSEQIRREITTMRLVAHKNIVQLHEVMATRNKIYFVMEYVKGGELFEKVAKRGKLTEVVAHKYFQQLISAVDYCHSRGVYHRDLKPENLLLDENENLKVSDFGLSALSESKRQDGLLHTTCGTPAYVAPEVISKIGYDGAKSDIWSCGVILFVLVAGYLPFQGPNLMEMYRKIQHGEFRCPGWFSRKLQKLLYKIMDPNPSTRISIQKIKESTWFRKDPEENRILKERTLNENTTKNVAPVLGVRRKKNAHEDVKPMSVTNLNAFEIISFSKGFDLSGMFIVKEWRNEARFTSDKSASTIISKLEDVAKALNLRVRKKDNGVVKMQGRKEGRNGVLQFDIEIFEVTTSYHIIEMKQTSGDSLEYRQLLEEGIRPALKDTVLA

SEQ ID NO: 3 - protein, 434 aa, Oryza glaberrima; identical to SEQ ID NO: 1

SEQ ID NO: 4 - protein, 405 aa, Oryza rufipogon
MESRGKILMERYELGRLLGKGTFGKVHYARNLESNQSVAIKMMDKQQILKVGLSEQIRREITTMRLVAHKNIVQLHEVMATRNKIYFVMEYVKGGELFEKVAKRGKLTEVVAHKYFQQLISAVDYCHSRGVYHRDLKPENLLLDENENLKVSDFGLSALSESKRQDGLLHTTCGTPAYVAPEVISKIGYDGAKSDIWSCGVILFVLVAGYLPFQGPNLMEMYRKIQHGEFRCPGWFSRKLQKLLYKIMDPNPSTRISIQKIKESTWFRKGPEENRILKERTLNENTTKNVAPVLGVRRKKNAHEDVKPMSVTNLNAFEIISFSKGFDLSGMFIVKEWRNEARFTSDKSASTIXILMSQAFSFRSSNSLGFSSNEFICLACVLFSIYVFVSGEFMLRQCMQLYLES

SEQ ID NO: 5 - protein, 444 aa, Brachypodium distachyon
MDSSGKVVLGRYEVGRLLGKGAFGKVHYAKNLKSNRSVAIKMLDKEKVLKVGLSEQVRREITTMRLVAHKNIVKLHEVMATRSKIYFVMEYVKGGQLFDKISNGGKLTESAAHKYFQQLISAVDYCHSRGVYHRDLKPENLLLDEHENLKVSDFGLSALSESKRQDGLLHTICGSPAYVAPEVISKGGYDGAKSDIWSCGVVLFVLVAGYLPFQGQNLMEMYMKIEQGDFKCPGWVSIKLRKLLHKIMDPDPSKRISIQKIKESTWFRKGPEETLTMERIPIENAPTNAAPTLMRRRKNSHGDAKPLAVTNLNAFEIISFSTGFDLSGLFVEKESKKETRFTSENPASAIISKLEDVAKALNLRVRKKDNGVVKMQGRKEGRNGVVQFDSEIFQITPSYHLIEMKQTSGDSLEYQKLLEDGIRPALKDIVWAWHGDDDHHQQRG

SEQ ID NO: 6 - protein, 444 aa, Setaria Italica
MEGRGKILMERYELGRMLGKGTFGKVHYAKNLESNQSVAIKMMDKDKVLKVGLSEQIRREITTMRLVAHKNIVELHEVMATRNKIYFVMEYVKGGELFEKIEKSGKLTEPAAHKYFQQLISAVDYCHSRGVYHRDLKPENLLLDEDENLKVSDFGLSALSESKRQDGLLHTTCGTPAYVAPEVISKIGYDGAKSDIWSCGVVLFVLVAGYLPFQGPNLMEMYRKVQHGDFRCPSWFSHKLKKLLYKILDPNPATRISIQKIKESTWFRKGPEGTRTVKEKIPCENATTNAAPTLSVRRKKNPYEDAKPLPVTNLNAFEIISFSSGFDLSGLFLEKECRKEARFTSDKSASAIILKIEDVAKMLNLRVRKKDNGVVKIQGRKEGRNGVLQFDTEIFEITPFHHLVEMKQTSGDFLEYQKLFEEDIRPALKDIVWAWHGDDQQQKQ

SEQ ID NO: 7 - protein, 444 aa, Setaria viridis
MEGRGKILMERYELGRMLGKGTFGKVHYAKNLESNQSVAIKMMDKDKVLKVGLSEQIRREITTMRLVAHKNIVELHEVMATRNKIYFVMEYVKGGELFEKIEKSGKLTEPAAHKYFQQLISAVDYCHSRGVYHRDLKPENLLLDEDENLKVSDFGLSALSESKRQDGLLHTTCGTPAYVAPEVISKIGYDGAKSDIWSCGVVLFVLVAGYLPFQGPNLMEMYRKVQHGDFRCPSWFSHKLKKLLYKILDPNPATRISIQKIKESTWFRKGPEGTRTVKEKIPCENATTNAAPTLSVRRKKNPYEDAKPLTVTNLNAFEIISFSSGFDLSGLFLEKECRKEARFTSDKSASAIILKIEDVAKMLNLRVRKKDNGVVKIQGRKEGRNGVLQFDTEIFEITPLHHLVEMKQTSGDFLEYQKLFEEDIRPALKDIVWAWHGDDQQQKQ

SEQ ID NO: 8 - protein, 443 aa, Sorghum bicolor
MESRGKILMKRYELGRLLGKGTFGKVHYARNLESNQSVAIKMMDKDKVLNVGLSEQIRREITTMRLVAHKNIVELHEVMATRNKIYFVMEYVKGGELFDKIEKSGKLTEAVAHKYFQQLISAVDYCHSQGVYHRDLKPENLLLDENENLKVSDFGLSALSESKRQDGLLHTTCGTPAYVAPEVISKTGYDGAKSDIWSCGVVLFVLVAGYLPFQGPNLMEMYRKIQNGNFRCPSWFSHKLKKLLYKILDPNPNTRISVQKIKESTWFRKGPGETRTVKEKIPSENATTNAAPVLAMRRKKNVHEDVKPLAVTNLNAFEIISFSTGFDLSGLFIKKECKKETRFTSDKPAAAIISKLEDVAKILNLRIRKMDNGVVKIQGRKEGRNGVLQFDAEIFEITASYHLIEMKQTGGDSLEYQKLLEENIRPALKDIVWAWHGDDQQQK

SEQ ID NO: 9 - protein, 440 aa, Hordeum vulgare
MENSGKIVMGRYELGRLLGKGAFGKVHYAKNLESNRGVAIKMMDKETVLKVGLAEQVRREITTMRLVAHKSIVQLHEVMATRSKIYFVMEYMKGGELFDKVSKAGKLTEGAAHKYFQQLISAVDYCHSRGVYHRDLKPENLLLDENENLKVSDFGLSALSESKRQDGLLHTTCGSPAYVAPEVISKGGYDGAKSDIWSCGVILFVLVAGYLPFQGQNLIEMYRKIEKGDFRCPGWVSPKLQKLLHKIMDPDPNKRISIQKIKESTWFRKGPGENLTVKERLPNENTTTDAVPTLGVRRRKNSHEDRKPLAVTNLNAFEIISFSTGFDLSGLFIEKESKKEARFTSEQPASAIVSKLEDVAKQLNLRVRKKDNGVVKMQVRKEGRNGVLQFDSEIFEISPSYHLIEMKQTSGDSLEYQKLLEEGIRPALKDIIWAWHGADG

SEQ ID NO: 10 - protein, 449 aa, Triticum aestivum
MQLEVMANRGKILMERYELGRLLGKGTFGKVHYARSLESNRSVAIKMLDKEKVLKVGLSEQIRREVTTMRLVAHKNIVQLHEVMATRNKIYFVMEYVKGGELFDKVAKSGKLTEGAAHKYFQQLISAVDYCHSQGVYHRDLKLENLLLDENENLKVSDFGLSALSESKRQDGLLHTTCGTPAYVAPEVISKTGYDGAKSDIWSCGVILFVLVAGYLPFHGSNLMDMYRKIEQGDFRCPSWFSHKLQKLLFKILDPNPSTRASIQKIKESTWFRKGPRGTLAVKERTPSENVTTNAPPTAGVRPRKNTHEDVKPLMVTNLNAFEIISFSTGFDLSGLFIREECRKETRFTSDKPASAIISKLEYVAKALNLRVRKKDNGVVKMQARKEGRNGAVQLDMEIFEITPSHHLIEMKQTSGDPLEYRELLEDIRPALKDIVWAWHGDDHHQQLE

SEQ ID NO: 11 - protein, 449 aa, Triticum dicoccoides
MQLEVMANRGKILMERYELGRLLGKGTFGKVHYARSLESNRSVAIKMLDKEKVLKVGLSEQIRREVTTMRLVAHKNIVQLHEVMATRNKIYFVMEYVKGGELFDKVAKSGKLTEGAAHKYFQQLISAVDYCHSQGVYHRDLKLENLLLDENENLKVSDFGLSALSESKRQDGLLHTTCGTPAYVAPEVISKTGYDGAKSDIWSCGVILFVLVAGYLPFHGSNLMDMYRKIEQGDFRCPSWFSHKLQKLLFKILDPNPSTRASIQKIKESTWFRKGPRGTLAVKERTPSENVTTNAPPTAGVRPRKNTHEDVKPLMVTNLNAFEIISFSTGFDLSGLFIREECRKETRFTSDKHASAIISKLEYVAKALNLRVRKKDNGVVKMQARKEGRNGAVQLDMEIFEITPSHHLIEMKQTSGDPLEYRELLEDIRPALKDIVWAWHGDDHQQQLE

SEQ ID NO: 12 - protein, 449 aa, Triticum turgidum; identical to SEQ ID NO: 10

SEQ ID NO: 13 - protein, 444 aa, Triticum urartu
MANRGKILMERYELGRLLGKGTFGKVHYARSLESNRSVAIKMLDKEKVLKVGLSEQIRREVTTMRLVAHKNIVQLHEVMATRNKIYFVMEYVKGGELFDKVAKSGKLTEGAAHKYFQQLISAVDYCHSQGVYHRDLKLENLLLDENENLKVSDFGLSALSESKRQDGLLHTTCGTPAYVAPEVISKTGYDGAKSDIWSCGVILFVLVAGYLPFHGSNLMDMYRKIEQGDFRCPSWFSHKLQKLLFKILDPNPSTRASIQKIKESTWFRKGPRGTLAVKERTPSENVTTNAPPTAGVRPRKNTHEDVKPLMVTNLNAFEIISFSTGFDLSGLFIQEECRKETRFTSDKPASAIISKLEYVAKALNLRVRKKDNGVVKMQARKEGRNGAVQLDMEIFEITPSHHLIEMKQTSGDPLEYRELLEDIRPALKDIVWAWHGDDHQQQLE

SEQ ID NO: 14 - protein, 443 aa, Zea mays
MESTGKILMERYEVGRLLGKGTFGKVHYAKNLESNESVAIKMMDKDKVLKAGLSEQIRREITTMRLVAHKNIVELHEVMATRNNIYFVMEYVKGGELFDKIGKSGKLTEAAAHKYFQQLIGAVDYCHSRGVYHRDLKPENLLLDENENLKVSDFGLSALSESKGQDGLLHTTCGTPAYVAPEVISKTGYDGAKSDIWSCGVVLFVLVAGYLPFRAPNMMEMYRKIQNGDFRCPSWFSHKLKKLLYKILDPSPDTRISVQKIKESTWFRKGPVETRTVKEKILSENDTTNAAPAVAMRRKKNVHEDVKPPTVTSLNAFEIISFSTGLDLSGLFIRKECKKETRFTSDKGAVSIISKLEDVAKALNLRVRKKDNGVVKIQGRKVGRKGVLEFDTEIFEITASYHLIEMKQTGGDSLEYKKLLEEDIRPALKDIVWAWHGDDQQQK

SEQ ID NO: 15 - protein, 436 aa, Leersia perrieri
METRGKILMERYELGRLLGKGTFGKVHYARNLESNQSVAIKMMDKQQALKVGLSEQIRREITTMRLVAHKNIVQLHEVMATRNKIYFVMEYVKGGELFEKVEKRGKLTEGVAHKYFQQLISAVDYCHSRGVYHRDLKLENLLLDENENLKVSDFGLSALSESRRQDGLLHTTCGTPAYVAPEVISKIGYDGAKSDIWSCGVILFVLVAGYLPFQGPNLMEMYRKIQHSEFRCPSWISRKLQKLLYKIMDPNPNTRISIQKIKESTWFRKGPEENRVLKERTLNESTTRNVAPTLGVRRKKNAHEDVKPMSVTNLNAFEIISFSTGFDLSGMFIAKERRNEARFTSDKSASTIISKMEDVAKVLNLRVRKKDNGVVKMQGRKEGRNGVLQFDMEIFEVTNSYHIIEMKQTNGDPLEYRQLLEEGIRPALKDIVLAWH

SEQ ID NO: 16 - protein, 445 aa, Saccharum spontaneum
MESRGKTLVERYEMGRLLGKGTFGKVHYGRNLKSNQSVAIKMMDKDKVLKVGLSEQIKREITTMRLVAHKNIVHLHEVMATRNKIYIVMEYVKGGELFDKIDKSGKLTEAAAHKYFQQLISAVDHCHSRGVYHRDLKPENLLLDENENLKVSDFGLSALSESKRQDGLLHTTCGTPAYVAPEVISKIGYDGAKSDIWSCGVVLFVLAAGYLPFQGPNLMEIYRKIQHGDFRCPSWFSHKLKKLLYKILDPNPSTRISIQKIKESTWFRKGPGEIRAVKEKILSENATTNAAPVLATRRKKIAHEDMKPLAATNLNAFEIISFSTGLDLSGLFIKKECRKETRFTSDKPALAIISKLEEVAKALNLRIRKKDNGIVKIQGRKEGRNGVLQFDTEIFEITPSYHLIEMKQTSGDSVEYQKLLEEDIRPGLKDIVWAWHGDDLQQKQE

SEQ ID NO: 17 - protein, 445 aa, Panicum hallii
MEGRGKILMERYELGRLLGKGTFGRVHYARNLESNRSVAIKMMDKDKVLKVGLSEQIRREITTMRLVAHKNIVELHEVMATRNKIYFVMEYVKGGELFDKIEKSGRLTESAAHNYFQQLISAVDYCHSRGVYHRDLKPENLLLDEDENLKVSDFGLSALSESKRQDGLLHTTCGTPAYVAPEVISKTGYDGAKSDIWSCGVVLFVLVAGYLPFQGPNLMEMYRKIQHGDFRCPSWFSHKLKKLLYKILDPNPSTRISIQKIKESTWFRKGPEGTRTVKEKIPSENATTNAAPTLSVRRGKNAYEDVKPLHVTNLNAFEIISFSSGFDLSGLFIEMEHRKEARFTSDKPASAIISKIEDVAKMLNLRVRKKDNGVVKIQGRKEGRNGVLQFDTEIFEITPFHHLVEMKQTSGDSLEYQKLFEQDIRPALKDIVWAWHGDDQQQMQE

SEQ ID NO: 18 - protein, 444 aa, Secale cereal
MANRGKILMERYELGRLLGKGTFGKVHYARSLESNQSVAIKMLDKEKVLKVGLSEQIRREVTTMRLVAHKNIVQLHEVMATRNKIYFVMEYVKGGELFDKVAKSGKLTEGVAHKYFQQLISAVDYCHSQGVYHRDLKLENLLLDENENLKVSDFGLSALSESKRQDGLLHTTCGTPAYVAPEVISKTGYDGAKSDIWSCGVILFVLVAGYLPFQGSNLMDMYRKIEQGDFRCPSWFSHKLQKLLCKILDPNPSTRASIKKIKESTWFRKGPRGTLAVKERTPSENVSTNTPPTAGVRPRKNTHEDVKPLMVTNLNAFEIISFSTGFDLSGLFIQEECRKVTRFTSDKPASAIISKLEYVAKAMNLRVRKKDNGVVKMQARKEGRNGAVQLDIEIFEITPSHHLIEMKQTSGDPLEYRELLEDIRPRLKDIVWAWHGDDHQQQLE

SEQ ID NO: 19 - protein, 444 aa, synthetic construct
MDSSGKVVLGRYEVGRLLGKGAFGKVHYAKNLKSNRSVAIKMLDKEKILKVGLSEQVRREITTMRLVAHKNIVKLHEVMATRSKIYFVMEYVKGGQLFDKISNGGKLTESAAHKYFQQLISAVDYCHSRGVYHRDLKPENLLLDEHENLKVSDFGLSALSESKRQDGLLHTICGSPAYVAPEVISKGGYDGAKSDIWSCGVVLFVLVAGYLPFQGQNLMEMYMKIEQGDFKCPGWVSIKLRKLLHKIMDPDPSKRISIQKIKESTWFRKGPEETLTMERIPIENAPTNAAPTLMRRRKNSHGDAKPLAVTNLNAFEIISFSTGFDLSGLFVEKESKKETRFTSENPASAIISKLEDVAKALNLRVRKKDNGVVKMQGRKEGRNGVVQFDSEIFQITPSYHLIEMKQTSGDSLEYQKLLEDGIRPALKDIVWAWHGDDDHHQQRG

SEQ ID NO: 20 - protein, 444 aa, synthetic construct
MEGRGKILMERYELGRMLGKGTFGKVHYAKNLESNQSVAIKMMDKDKILKVGLSEQIRREITTMRLVAHKNIVELHEVMATRNKIYFVMEYVKGGELFEKIEKSGKLTEPAAHKYFQQLISAVDYCHSRGVYHRDLKPENLLLDEDENLKVSDFGLSALSESKRQDGLLHTTCGTPAYVAPEVISKIGYDGAKSDIWSCGVVLFVLVAGYLPFQGPNLMEMYRKVQHGDFRCPSWFSHKLKKLLYKILDPNPATRISIQKIKESTWFRKGPEGTRTVKEKIPCENATTNAAPTLSVRRKKNPYEDAKPLPVTNLNAFEIISFSSGFDLSGLFLEKECRKEARFTSDKSASAIILKIEDVAKMLNLRVRKKDNGVVKIQGRKEGRNGVLQFDTEIFEITPFHHLVEMKQTSGDFLEYQKLFEEDIRPALKDIVWAWHGDDQQQKQ

SEQ ID NO: 21 - protein, 444 aa, synthetic construct
MEGRGKILMERYELGRMLGKGTFGKVHYAKNLESNQSVAIKMMDKDKILKVGLSEQIRREITTMRLVAHKNIVELHEVMATRNKIYFVMEYVKGGELFEKIEKSGKLTEPAAHKYFQQLISAVDYCHSRGVYHRDLKPENLLLDEDENLKVSDFGLSALSESKRQDGLLHTTCGTPAYVAPEVISKIGYDGAKSDIWSCGVVLFVLVAGYLPFQGPNLMEMYRKVQHGDFRCPSWFSHKLKKLLYKILDPNPATRISIQKIKESTWFRKGPEGTRTVKEKIPCENATTNAAPTLSVRRKKNPYEDAKPLTVTNLNAFEIISFSSGFDLSGLFLEKECRKEARFTSDKSASAIILKIEDVAKMLNLRVRKKDNGVVKIQGRKEGRNGVLQFDTEIFEITPLHHLVEMKQTSGDFLEYQKLFEEDIRPALKDIVWAWHGDDQQQKQ

SEQ ID NO: 22 - protein, 443 aa, synthetic construct
MESRGKILMKRYELGRLLGKGTFGKVHYARNLESNQSVAIKMMDKDKILNVGLSEQIRREITTMRLVAHKNIVELHEVMATRNKIYFVMEYVKGGELFDKIEKSGKLTEAVAHKYFQQLISAVDYCHSQGVYHRDLKPENLLLDENENLKVSDFGLSALSESKRQDGLLHTTCGTPAYVAPEVISKTGYDGAKSDIWSCGVVLFVLVAGYLPFQGPNLMEMYRKIQNGNFRCPSWFSHKLKKLLYKILDPNPNTRISVQKIKESTWFRKGPGETRTVKEKIPSENATTNAAPVLAMRRKKNVHEDVKPLAVTNLNAFEIISFSTGFDLSGLFIKKECKKETRFTSDKPAAAIISKLEDVAKILNLRIRKMDNGVVKIQGRKEGRNGVLQFDAEIFEITASYHLIEMKQTGGDSLEYQKLLEENIRPALKDIVWAWHGDDQQQK

SEQ ID NO: 23 - protein, 440 aa, synthetic construct
MENSGKIVMGRYELGRLLGKGAFGKVHYAKNLESNRGVAIKMMDKETILKVGLAEQVRREITTMRLVAHKSIVQLHEVMATRSKIYFVMEYMKGGELFDKVSKAGKLTEGAAHKYFQQLISAVDYCHSRGVYHRDLKPENLLLDENENLKVSDFGLSALSESKRQDGLLHTTCGSPAYVAPEVISKGGYDGAKSDIWSCGVILFVLVAGYLPFQGQNLIEMYRKIEKGDFRCPGWVSPKLQKLLHKIMDPDPNKRISIQKIKESTWFRKGPGENLTVKERLPNENTTTDAVPTLGVRRRKNSHEDRKPLAVTNLNAFEIISFSTGFDLSGLFIEKESKKEARFTSEQPASAIVSKLEDVAKQLNLRVRKKDNGVVKMQVRKEGRNGVLQFDSEIFEISPSYHLIEMKQTSGDSLEYQKLLEEGIRPALKDIIWAWHGADG

SEQ ID NO: 24 - protein, 449 aa, synthetic construct
MQLEVMANRGKILMERYELGRLLGKGTFGKVHYARSLESNRSVAIKMLDKEKILKVGLSEQIRREVTTMRLVAHKNIVQLHEVMATRNKIYFVMEYVKGGELFDKVAKSGKLTEGAAHKYFQQLISAVDYCHSQGVYHRDLKLENLLLDENENLKVSDFGLSALSESKRQDGLLHTTCGTPAYVAPEVISKTGYDGAKSDIWSCGVILFVLVAGYLPFHGSNLMDMYRKIEQGDFRCPSWFSHKLQKLLFKILDPNPSTRASIQKIKESTWFRKGPRGTLAVKERTPSENVTTNAPPTAGVRPRKNTHEDVKPLMVTNLNAFEIISFSTGFDLSGLFIREECRKETRFTSDKPASAIISKLEYVAKALNLRVRKKDNGVVKMQARKEGRNGAVQLDMEIFEITPSHHLIEMKQTSGDPLEYRELLEDIRPALKDIVWAWHGDDHHQQLE

SEQ ID NO: 25 - protein, 449 aa, synthetic construct
MQLEVMANRGKILMERYELGRLLGKGTFGKVHYARSLESNRSVAIKMLDKEKILKVGLSEQIRREVTTMRLVAHKNIVQLHEVMATRNKIYFVMEYVKGGELFDKVAKSGKLTEGAAHKYFQQLISAVDYCHSQGVYHRDLKLENLLLDENENLKVSDFGLSALSESKRQDGLLHTTCGTPAYVAPEVISKTGYDGAKSDIWSCGVILFVLVAGYLPFHGSNLMDMYRKIEQGDFRCPSWFSHKLQKLLFKILDPNPSTRASIQKIKESTWFRKGPRGTLAVKERTPSENVTTNAPPTAGVRPRKNTHEDVKPLMVTNLNAFEIISFSTGFDLSGLFIREECRKETRFTSDKHASAIISKLEYVAKALNLRVRKKDNGVVKMQARKEGRNGAVQLDMEIFEITPSHHLIEMKQTSGDPLEYRELLEDIRPALKDIVWAWHGDDHQQQLE

SEQ ID NO: 26 - protein, 449 aa, synthetic construct; identical to SEQ ID NO: 24

SEQ ID NO: 27 - protein, 444 aa, synthetic construct
MANRGKILMERYELGRLLGKGTFGKVHYARSLESNRSVAIKMLDKEKILKVGLSEQIRREVTTMRLVAHKNIVQLHEVMATRNKIYFVMEYVKGGELFDKVAKSGKLTEGAAHKYFQQLISAVDYCHSQGVYHRDLKLENLLLDENENLKVSDFGLSALSESKRQDGLLHTTCGTPAYVAPEVISKTGYDGAKSDIWSCGVILFVLVAGYLPFHGSNLMDMYRKIEQGDFRCPSWFSHKLQKLLFKILDPNPSTRASIQKIKESTWFRKGPRGTLAVKERTPSENVTTNAPPTAGVRPRKNTHEDVKPLMVTNLNAFEIISFSTGFDLSGLFIQEECRKETRFTSDKPASAIISKLEYVAKALNLRVRKKDNGVVKMQARKEGRNGAVQLDMEIFEITPSHHLIEMKQTSGDPLEYRELLEDIRPALKDIVWAWHGDDHQQQLE

SEQ ID NO: 28 - protein, 443 aa, synthetic construct
MESTGKILMERYEVGRLLGKGTFGKVHYAKNLESNESVAIKMMDKDKILKAGLSEQIRREITTMRLVAHKNIVELHEVMATRNNIYFVMEYVKGGELFDKIGKSGKLTEAAAHKYFQQLIGAVDYCHSRGVYHRDLKPENLLLDENENLKVSDFGLSALSESKGQDGLLHTTCGTPAYVAPEVISKTGYDGAKSDIWSCGVVLFVLVAGYLPFRAPNMMEMYRKIQNGDFRCPSWFSHKLKKLLYKILDPSPDTRISVQKIKESTWFRKGPVETRTVKEKILSENDTTNAAPAVAMRRKKNVHEDVKPPTVTSLNAFEIISFSTGLDLSGLFIRKECKKETRFTSDKGAVSIISKLEDVAKALNLRVRKKDNGVVKIQGRKVGRKGVLEFDTEIFEITASYHLIEMKQTGGDSLEYKKLLEEDIRPALKDIVWAWHGDDQQQK

SEQ ID NO: 29 - protein, 436 aa, synthetic construct
METRGKILMERYELGRLLGKGTFGKVHYARNLESNQSVAIKMMDKQQILKVGLSEQIRREITTMRLVAHKNIVQLHEVMATRNKIYFVMEYVKGGELFEKVEKRGKLTEGVAHKYFQQLISAVDYCHSRGVYHRDLKLENLLLDENENLKVSDFGLSALSESRRQDGLLHTTCGTPAYVAPEVISKIGYDGAKSDIWSCGVILFVLVAGYLPFQGPNLMEMYRKIQHSEFRCPSWISRKLQKLLYKIMDPNPNTRISIQKIKESTWFRKGPEENRVLKERTLNESTTRNVAPTLGVRRKKNAHEDVKPMSVTNLNAFEIISFSTGFDLSGMFIAKERRNEARFTSDKSASTIISKMEDVAKVLNLRVRKKDNGVVKMQGRKEGRNGVLQFDMEIFEVTNSYHIIEMKQTNGDPLEYRQLLEEGIRPALKDIVLAWH

SEQ ID NO: 30 - protein, 445 aa, synthetic construct
MESRGKTLVERYEMGRLLGKGTFGKVHYGRNLKSNQSVAIKMMDKDKILKVGLSEQIKREITTMRLVAHKNIVHLHEVMATRNKIYIVMEYVKGGELFDKIDKSGKLTEAAAHKYFQQLISAVDHCHSRGVYHRDLKPENLLLDENENLKVSDFGLSALSESKRQDGLLHTTCGTPAYVAPEVISKIGYDGAKSDIWSCGVVLFVLAAGYLPFQGPNLMEIYRKIQHGDFRCPSWFSHKLKKLLYKILDPNPSTRISIQKIKESTWFRKGPGEIRAVKEKILSENATTNAAPVLATRRKKIAHEDMKPLAATNLNAFEIISFSTGLDLSGLFIKKECRKETRFTSDKPALAIISKLEEVAKALNLRIRKKDNGIVKIQGRKEGRNGVLQFDTEIFEITPSYHLIEMKQTSGDSVEYQKLLEEDIRPGLKDIVWAWHGDDLQQKQE

SEQ ID NO: 31 - protein, 445 aa, synthetic construct
MEGRGKILMERYELGRLLGKGTFGRVHYARNLESNRSVAIKMMDKDKILKVGLSEQIRREITTMRLVAHKNIVELHEVMATRNKIYFVMEYVKGGELFDKIEKSGRLTESAAHNYFQQLISAVDYCHSRGVYHRDLKPENLLLDEDENLKVSDFGLSALSESKRQDGLLHTTCGTPAYVAPEVISKTGYDGAKSDIWSCGVVLFVLVAGYLPFQGPNLMEMYRKIQHGDFRCPSWFSHKLKKLLYKILDPNPSTRISIQKIKESTWFRKGPEGTRTVKEKIPSENATTNAAPTLSVRRGKNAYEDVKPLHVTNLNAFEIISFSSGFDLSGLFIEMEHRKEARFTSDKPASAIISKIEDVAKMLNLRVRKKDNGVVKIQGRKEGRNGVLQFDTEIFEITPFHHLVEMKQTSGDSLEYQKLFEQDIRPALKDIVWAWHGDDQQQMQE

SEQ ID NO: 32 - protein, 444 aa, synthetic construct
MANRGKILMERYELGRLLGKGTFGKVHYARSLESNQSVAIKMLDKEKILKVGLSEQIRREVTTMRLVAHKNIVQLHEVMATRNKIYFVMEYVKGGELFDKVAKSGKLTEGVAHKYFQQLISAVDYCHSQGVYHRDLKLENLLLDENENLKVSDFGLSALSESKRQDGLLHTTCGTPAYVAPEVISKTGYDGAKSDIWSCGVILFVLVAGYLPFQGSNLMDMYRKIEQGDFRCPSWFSHKLQKLLCKILDPNPSTRASIKKIKESTWFRKGPRGTLAVKERTPSENVSTNTPPTAGVRPRKNTHEDVKPLMVTNLNAFEIISFSTGFDLSGLFIQEECRKVTRFTSDKPASAIISKLEYVAKAMNLRVRKKDNGVVKMQARKEGRNGAVQLDIEIFEITPSHHLIEMKQTSGDPLEYRELLEDIRPRLKDIVWAWHGDDHQQQLE

SEQ ID NO: 33 - protein, 439 aa, Oryza sativa
MESRGKILMERYELGRLLGKGTFGKVHYARNLESNQSVAIKMMDKQQVLKVGLSEQIRREITTMRLVAHKNIVQLHEVMATRNKIYFVMEYVKGGELFEKVAKRGKLTEVVAHKYFQQLISAVDYCHSRGVYHRDLKPENLLLDENENLKVSDFGLSALSESKRQDGLLHTTCGTPAYVAPEVISKIGYDGAKSDIWSCGVILFVLVAGYLPFQGPNLMEMYRKIQHGEFRCPGWFSRKLQKLLYKIMDPNPSTRISIQKIKESTWFRKGPEENRILKERTLNENTTKNVALVLGVRRKKNAHEDVKPMSVTNLNAFEIISFSKGFDLSGMFIVKEWRNEARFTSDKSASTIISKLEDVAKALNLRVRKKDNGVVKMQGRKEGRNGVLQFDIEIFEVTTSYHIIEMKQTSGDSLEYRQLLEEGIRPALKDIVLAWHGDE

SEQ ID NO: 34 - DNA, 1986 bp, Zea mays
  source 1..1986: mol_type = genomic DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgca

SEQ ID NO: 35 - DNA, 3557 bp, synthetic construct
  source 1..3557: mol_type = other DNA
gtcattcatatgcttgagaagagagtcgggatagtccaaaataaaacaaaggtaagattacctggtcaaaagtgaaaacatcagttaaaaggtggtataagtaaaatatcggtaataaaaggtggcccaaagtgaaatttactcttttctactattataaaaattgaggatgttttgtcggtactttgatacgtcatttttgtatgaattggtttttaagtttattcgcgatttggaaatgcatatctgtatttgagtcggtttttaagttcgttgcttttgtaaatacagagggatttgtataagaaatatctttaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtgcagccaactggcaaatagtctccacccccggcactatcaccgtgagttgtccgcaccaccgcacgtctcgcagccaaaaaaaaaaaaagaaagaaaaaaaagaaaaagaaaaacagcaggtgggtccgggtcgtgggggccggaaaagcgaggaggatcgcgagcagcgacgaggcccggccctccctccgcttccaaagaaacgccccccatcgccactatatacatacccccccctctcctcccatccccccaaccctaccaccaccaccaccaccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtaaatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagatgaaagcgttaacggccaggcaacaagaggtgtttgatctcatccgtgatcacatcagccagacaggtatgccgccgacgcgtgcggaaatcgcgcagcgtttggggttccgttccccaaacgcggctgaagaacatctgaaggcgctggcacgcaaaggcgttattgaaattgtttccggcgcatcacgcgggattcgtctgttgcaggaagaggaagaagggttgccgctggtaggtcgtgtggctgccggtgaaccgtcgagcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatccgtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgacagagatgttagctgaaatcatcactaatcagataccaaaatattcaaatggaaatatcaaaaagcttctgtttcatcaaaaatgactcgacctaattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacatcccaaacagcttgggctgcaggtcgaggctaaaaaactaatcgcattatcatcccctcgacgtactgtacatataaccactggttttatatacagcagtactgtacatataaccactggttttatatacagcagtcgacgtactgtacatataaccactggttttatatacagcagtactgtacatataaccactggttttatatacagcagtcgaggtaagattagatatggatatgtatatggatatgtatatggtggtaatgccatgtaatatgcgacccttcctctatataaggaagttcatttcatttggagaggacacgctgaagctagtc

SEQ ID NO: 36 - DNA, 3435 bp, Oryza sativa
  source 1..3435: mol_type = genomic DNA
ctccaatcctccctcccttcctcccagatccgtctaccagcggtggaggcgacagtggcgggggaggagccgacggcggcggccaccgcagagagcgtcgccccatcccctcctagatctggacggaagaggcgagccagccggcagcagcggcggacggaggaggagagccatcggtcggagggggaagggccagatggggaggctagcgccggcgccagcgccggcaggtaggggtggtggcggcggattttttttgttcgctgaaaatatttttgcaggtgggtggatctaacgtctgtaaaaatcatctattttcgtaagcctttgcttacaagcggtcatttctcccgcctggaaaaaatatttttggcctcttgggaaaatgattttcctagtagtgtgggtgtttatatttatggctaattattattttagtggtaggtgacgtactcaccgacagcgagacgcccgcgatgacttcgttaatttcaaaatatatcggcccagtctttcggatgtgctcctagggatagggtgtacgtgtctatgtttgtagaggtgaatgtacgcttgttgttaaaaaattgggttgcttggtttgattgtatgagtctgataagtaattgttttttttttgttttttttttgttggaaggaataggacatgtaacgattcgtgctgtatgtggcaccacgacgctgttgccattgcaacgtaaggtgtacattcaccagcttccctttccgttttcgagcagcaaattccaaatgtaattccttttgtcacacgcaggcaacacaacgcatggactccacatatcgtgtgcagataattaattttggacaaagacatttcttggaagtggcacagattttttttcgctctcatgaaaggcttgattgatgcaaaccaattgagccgtcatcggctatctgtacattatctgtacagctgccaagtttgtattgatcggttttctttgaggttaatttgtaagtatgtttttcttctagtaacttaagtaattatttttaaatctaaggtcaatatgcataacaacatattctgaaaaacttatggataagctgattattcattggtaaaatatagacaggccgatgtcaataaaaaccaatgatgcctgaccgaaacaaatactacatgtctacatccatgctaaaattaaataagttcagttctatacatgaatttgactatattcatatttatgtaaaaaataaatttatttttgaatggaggaaataagtatatacggtgataattgaataccgctcaccaaagatccagctagtagcacacggataaatggctgcaagtcaccaaccatttccggtggtccaatacctgaagaacagccaacccattgatgagtcagcaaaggatcagatcatgagagatgagccaatgccatgcatttgagtaactccaaaccaaattaaacacaacatcacatggaaaactactctaacaaatgcctcaattaacattggttcctcagaaaataaaagttgcagttaacggaatcagattaaagaaggaaacaagtgctcattttctgtaagacagcatcacagccttcagatctataaataggattaggcaaggtgattagtggtacattatgtgctgtacctgtttcaagaacacaatgatgagctgtaccatcatcaattgtgaaataattaattaaattatcattaagccccaagccaagccaaaccaatccaagccaagccaaatgaatgattgctgtggtgaagggcgactagtcaggggtgagtcggatggatatgctctttatgcgatgcggtgcccaggaaacgcggcgcagccaaatcagggtcattaacgtcaaatcaatctctgctaataatgcaaggcaccactgtttcttctccaactggcggctgcctcctctcctcttcttgatcggttccccctgtataaaagagaaccccgtcttctcgctgtcctcgcatccactcctgatactaccactcaaaaactctctctctctacccaaacaggtgttgagaaagaagtgagggacagatccaaaaagttcaccttttgttacctgaatctcctcttcttcctgtcaccggaaggtatgcagttcctctctccatatcatcttctagcgtttctttctactgattcttgattgctcgcttttttccattattctcttttgctccaaggtaggggtaggggcacatcagttggacaagtagaaattgatggcacatcaattgagtagatgcggaaatttatggtttttagttaaagtttagaccaagccgatgacaaagtgctgccgtttaatcgaatacgacgatatgtactcttgcaatggcatgtgtcatgaggtgtttgggtttggcgaggctcagttttcattaatttcccctccttcccatggccgtatcaaacctaatctttgaaaaatattcaattcatgtgggtgcggaatcactagatgcagcttaactccatgcaaatcctaactcctgccatgtgcccatgtgcctgtcccttatcctgatttgcatctgaattcatttgctgagtaatttgcttgttcagtgcctaatctgctagacgtccaaacaaagttttaattatgagcaaaagatctagtaatgtcattttttctaaattcgtaatgttttggtctctgaattcgtaattgactagttttattaatcacctttctgaacttgaacgaaagatgtagaataattcacatcggttgcaaattttaattcgtaattgactagttttattttgttagcttctgatttactgtcctagatttctgataagtacttagttgaagttccttattatattaattatctattacataacacgttgaactcatgtaagttctgattgattctagggaacacttatattatataagtagtaggtacaaaaatgcatttagtattacaataagccttcaaaattcttcgcattaacttttgtttggttaaagttcttacagactttcagattattatacgtaccttgttagcacttgagtgcatatcctaggacattcaattactaaatctgatgcctggttttgtttatacaggggaatcaataaaaaaaacaagcagtgaaagcctcaaggaatgctgaaagatgtgcttgcatagaagaaattttccgtgtgagataaaacagattgctggttcaccagtgctactgatctactcaagtcttgatgtgagtgatcaactttcctgcactaattctacaccatttagactccgaaacgacgatgatataagaatgttgttctccatccacgatcatgaccttggggaggatggctaaagaattgcagtccatctgattggtggtcaaaacatagtgctgagatttatatctttgtgggc

SEQ ID NO: 37 - DNA, 3571 bp, synthetic construct
  source 1..3571: mol_type = other DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaaatggagagtagagggaagattctaatggagaggtatgagttggggagattgttggggaaaggaacatttggcaaggtgcactatgcaaggaatctggagtcaaaccagagtgtggccataaagatgatggacaaacagcagatattgaaggtcgggctttcggagcagatcagacgtgagatcacaaccatgcggttggtggctcataagaacattgttcagcttcatgaggtcatggcaacacggaacaagatctactttgtgatggagtatgtgaaaggtggtgagctatttgaaaaggttgcaaagcgtggaaagcttacagaggttgttgcacataagtatttccagcaactcattagtgcagtggattactgccacagtcgaggtgtgtatcaccgggacttgaagcctgagaacctactgttggatgagaatgagaacctgaaagtctcagactttggattgagtgcgctttcagagtcgaagaggcaagatggcttactccataccacctgtggaacacctgcatatgtagctccagaggtgattagcaagataggctatgatggtgcaaagtcagatatttggtcttgtggtgttatcctgtttgttcttgttgctggttaccttcgtttccagggcccaaacttgatggaaatgtatcggaagatacaacacggtgaattcaggtgccccggttggttttcacgcaaactccagaagttgttgtacaagatcatggaccccaacccaagcacaaggatttcaatccagaagataaaggagtctacctggttccggaaaggtcctgaggagaaccgtattttgaaggaaagaactttgaatgaaaacaccaccaaaaatgttgctccggtgcttggtgtgagacgcaagaaaaatgctcatgaagatgtgaagcccatgtcagtgacaaacttaaatgcttttgaaattatctctttctctaagggatttgatctctctggcatgttcattgtaaaggaatggagaaatgaggcaaggttcacttcagataaatctgcctcaaccataatctcaaagctagaagatgtagcaaaggcgctaaatctcagggtaaggaagaaagacaatggtgtagtgaagatgcaagggaggaaggagggaaggaatggtgttcttcagtttgacatagagatatttgaggttaccacttcctatcatatcatcgagatgaaacaaacaagtggcgattcattggagtaccgacagctactggaggagggcatccggccagctctgaaggacattgtcttggcctgatgaactagtggatcccccgggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 38 - DNA, 5114 bp, synthetic construct
  source 1..5114: mol_type = other DNA
gtcattcatatgcttgagaagagagtcgggatagtccaaaataaaacaaaggtaagattacctggtcaaaagtgaaaacatcagttaaaaggtggtataagtaaaatatcggtaataaaaggtggcccaaagtgaaatttactcttttctactattataaaaattgaggatgttttgtcggtactttgatacgtcatttttgtatgaattggtttttaagtttattcgcgatttggaaatgcatatctgtatttgagtcggtttttaagttcgttgcttttgtaaatacagagggatttgtataagaaatatctttaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtgcagccaactggcaaatagtctccacccccggcactatcaccgtgagttgtccgcaccaccgcacgtctcgcagccaaaaaaaaaaaaagaaagaaaaaaaagaaaaagaaaaacagcaggtgggtccgggtcgtgggggccggaaaagcgaggaggatcgcgagcagcgacgaggcccggccctccctccgcttccaaagaaacgccccccatcgccactatatacatacccccccctctcctcccatccccccaaccctaccaccaccaccaccaccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtaaatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagatgaaagcgttaacggccaggcaacaagaggtgtttgatctcatccgtgatcacatcagccagacaggtatgccgccgacgcgtgcggaaatcgcgcagcgtttggggttccgttccccaaacgcggctgaagaacatctgaaggcgctggcacgcaaaggcgttattgaaattgtttccggcgcatcacgcgggattcgtctgttgcaggaagaggaagaagggttgccgctggtaggtcgtgtggctgccggtgaaccgtcgagcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatccgtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgacagagatgttagctgaaatcatcactaatcagataccaaaatattcaaatggaaatatcaaaaagcttctgtttcatcaaaaatgactcgacctaattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacatcccaaacagcttgggctgcaggtcgaggctaaaaaactaatcgcattatcatcccctcgacgtactgtacatataaccactggttttatatacagcagtactgtacatataaccactggttttatatacagcagtcgacgtactgtacatataaccactggttttatatacagcagtactgtacatataaccactggttttatatacagcagtcgaggtaagattagatatggatatgtatatggatatgtatatggtggtaatgccatgtaatatgcgacccttcctctatataaggaagttcatttcatttggagaggacacgctgaagctagtcatggagagtagagggaagattctaatggagaggtatgagttggggagattgttggggaaaggaacatttggcaaggtgcactatgcaaggaatctggagtcaaaccagagtgtggccataaagatgatggacaaacagcagatattgaaggtcgggctttcggagcagatcagacgtgagatcacaaccatgcggttggtggctcataagaacattgttcagcttcatgaggtcatggcaacacggaacaagatctactttgtgatggagtatgtgaaaggtggtgagctatttgaaaaggttgcaaagcgtggaaagcttacagaggttgttgcacataagtatttccagcaactcattagtgcagtggattactgccacagtcgaggtgtgtatcaccgggacttgaagcctgagaacctactgttggatgagaatgagaacctgaaagtctcagactttggattgagtgcgctttcagagtcgaagaggcaagatggcttactccataccacctgtggaacacctgcatatgtagctccagaggtgattagcaagataggctatgatggtgcaaagtcagatatttggtcttgtggtgttatcctgtttgttcttgttgctggttaccttcgtttccagggcccaaacttgatggaaatgtatcggaagatacaacacggtgaattcaggtgccccggttggttttcacgcaaactccagaagttgttgtacaagatcatggaccccaacccaagcacaaggatttcaatccagaagataaaggagtctacctggttccggaaaggtcctgaggagaaccgtattttgaaggaaagaactttgaatgaaaacaccaccaaaaatgttgctccggtgcttggtgtgagacgcaagaaaaatgctcatgaagatgtgaagcccatgtcagtgacaaacttaaatgcttttgaaattatctctttctctaagggatttgatctctctggcatgttcattgtaaaggaatggagaaatgaggcaaggttcacttcagataaatctgcctcaaccataatctcaaagctagaagatgtagcaaaggcgctaaatctcagggtaaggaagaaagacaatggtgtagtgaagatgcaagggaggaaggagggaaggaatggtgttcttcagtttgacatagagatatttgaggttaccacttcctatcatatcatcgagatgaaacaaacaagtggcgattcattggagtaccgacagctactggaggagggcatccggccagctctgaaggacattgtcttggcctgaatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 39 - DNA, 5382 bp, synthetic construct
  source 1..5382: mol_type = other DNA
ctccaatcctccctcccttcctcccagatccgtctaccagcggtggaggcgacagtggcgggggaggagccgacggcggcggccaccgcagagagcgtcgccccatcccctcctagatctggacggaagaggcgagccagccggcagcagcggcggacggaggaggagagccatcggtcggagggggaagggccagatggggaggctagcgccggcgccagcgccggcaggtaggggtggtggcggcggattttttttgttcgctgaaaatatttttgcaggtgggtggatctaacgtctgtaaaaatcatctattttcgtaagcctttgcttacaagcggtcatttctcccgcctggaaaaaatatttttggcctcttgggaaaatgattttcctagtagtgtgggtgtttatatttatggctaattattattttagtggtaggtgacgtactcaccgacagcgagacgcccgcgatgacttcgttaatttcaaaatatatcggcccagtctttcggatgtgctcctagggatagggtgtacgtgtctatgtttgtagaggtgaatgtacgcttgttgttaaaaaattgggttgcttggtttgattgtatgagtctgataagtaattgttttttttttgttttttttttgttggaaggaataggacatgtaacgattcgtgctgtatgtggcaccacgacgctgttgccattgcaacgtaaggtgtacattcaccagcttccctttccgttttcgagcagcaaattccaaatgtaattccttttgtcacacgcaggcaacacaacgcatggactccacatatcgtgtgcagataattaattttggacaaagacatttcttggaagtggcacagattttttttcgctctcatgaaaggcttgattgatgcaaaccaattgagccgtcatcggctatctgtacattatctgtacagctgccaagtttgtattgatcggttttctttgaggttaatttgtaagtatgtttttcttctagtaacttaagtaattatttttaaatctaaggtcaatatgcataacaacatattctgaaaaacttatggataagctgattattcattggtaaaatatagacaggccgatgtcaataaaaaccaatgatgcctgaccgaaacaaatactacatgtctacatccatgctaaaattaaataagttcagttctatacatgaatttgactatattcatatttatgtaaaaaataaatttatttttgaatggaggaaataagtatatacggtgataattgaataccgctcaccaaagatccagctagtagcacacggataaatggctgcaagtcaccaaccatttccggtggtccaatacctgaagaacagccaacccattgatgagtcagcaaaggatcagatcatgagagatgagccaatgccatgcatttgagtaactccaaaccaaattaaacacaacatcacatggaaaactactctaacaaatgcctcaattaacattggttcctcagaaaataaaagttgcagttaacggaatcagattaaagaaggaaacaagtgctcattttctgtaagacagcatcacagccttcagatctataaataggattaggcaaggtgattagtggtacattatgtgctgtacctgtttcaagaacacaatgatgagctgtaccatcatcaattgtgaaataattaattaaattatcattaagccccaagccaagccaaaccaatccaagccaagccaaatgaatgattgctgtggtgaagggcgactagtcaggggtgagtcggatggatatgctctttatgcgatgcggtgcccaggaaacgcggcgcagccaaatcagggtcattaacgtcaaatcaatctctgctaataatgcaaggcaccactgtttcttctccaactggcggctgcctcctctcctcttcttgatcggttccccctgtataaaagagaaccccgtcttctcgctgtcctcgcatccactcctgatactaccactcaaaaactctctctctctacccaaacaggtgttgagaaagaagtgagggacagatccaaaaagttcaccttttgttacctgaatctcctcttcttcctgtcaccggaaggtatgcagttcctctctccatatcatcttctagcgtttctttctactgattcttgattgctcgcttttttccattattctcttttgctccaaggtaggggtaggggcacatcagttggacaagtagaaattgatggcacatcaattgagtagatgcggaaatttatggtttttagttaaagtttagaccaagccgatgacaaagtgctgccgtttaatcgaatacgacgatatgtactcttgcaatggcatgtgtcatgaggtgtttgggtttggcgaggctcagttttcattaatttcccctccttcccatggccgtatcaaacctaatctttgaaaaatattcaattcatgtgggtgcggaatcactagatgcagcttaactccatgcaaatcctaactcctgccatgtgcccatgtgcctgtcccttatcctgatttgcatctgaattcatttgctgagtaatttgcttgttcagtgcctaatctgctagacgtccaaacaaagttttaattatgagcaaaagatctagtaatgtcattttttctaaattcgtaatgttttggtctctgaattcgtaattgactagttttattaatcacctttctgaacttgaacgaaagatgtagaataattcacatcggttgcaaattttaattcgtaattgactagttttattttgttagcttctgatttactgtcctagatttctgataagtacttagttgaagttccttattatattaattatctattacataacacgttgaactcatgtaagttctgattgattctagggaacacttatattatataagtagtaggtacaaaaatgcatttagtattacaataagccttcaaaattcttcgcattaacttttgtttggttaaagttcttacagactttcagattattatacgtaccttgttagcacttgagtgcatatcctaggacattcaattactaaatctgatgcctggttttgtttatacaggggaatcaataaaaaaaacaagcagtgaaagcctcaaggaatgctgaaagatgtgcttgcatagaagaaattttccgtgtgagataaaacagattgctggttcaccagtgctactgatctactcaagtcttgatgtgagtgatcaactttcctgcactaattctacaccatttagactccgaaacgacgatgatataagaatgttgttctccatccacgatcatgaccttggggaggatggctaaagaattgcagtccatctgattggtggtcaaaacatagtgctgagatttatatctttgtgggcatggagagtagagggaagattctaatggagaggtatgagttggggagattgttggggaaaggaacatttggcaaggtgcactatgcaaggaatctggagtcaaaccagagtgtggccataaagatgatggacaaacagcagatattgaaggtcgggctttcggagcagatcagacgtgagatcacaaccatgcggttggtggctcataagaacattgttcagcttcatgaggtcatggcaacacggaacaagatctactttgtgatggagtatgtgaaaggtggtgagctatttgaaaaggttgcaaagcgtggaaagcttacagaggttgttgcacataagtatttccagcaactcattagtgcagtggattactgccacagtcgaggtgtgtatcaccgggacttgaagcctgagaacctactgttggatgagaatgagaacctgaaagtctcagactttggattgagtgcgctttcagagtcgaagaggcaagatggcttactccataccacctgtggaacacctgcatatgtagctccagaggtgattagcaagataggctatgatggtgcaaagtcagatatttggtcttgtggtgttatcctgtttgttcttgttgctggttaccttcgtttccagggcccaaacttgatggaaatgtatcggaagatacaacacggtgaattcaggtgccccggttggttttcacgcaaactccagaagttgttgtacaagatcatggaccccaacccaagcacaaggatttcaatccagaagataaaggagtctacctggttccggaaaggtcctgaggagaaccgtattttgaaggaaagaactttgaatgaaaacaccaccaaaaatgttgctccggtgcttggtgtgagacgcaagaaaaatgctcatgaagatgtgaagcccatgtcagtgacaaacttaaatgcttttgaaattatctctttctctaagggatttgatctctctggcatgttcattgtaaaggaatggagaaatgaggcaaggttcacttcagataaatctgcctcaaccataatctcaaagctagaagatgtagcaaaggcgctaaatctcagggtaaggaagaaagacaatggtgtagtgaagatgcaagggaggaaggagggaaggaatggtgttcttcagtttgacatagagatatttgaggttaccacttcctatcatatcatcgagatgaaacaaacaagtggcgattcattggagtaccgacagctactggaggagggcatccggccagctctgaaggacattgtcttggcctgaagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaa